(19) United States
(12) Patent Application Publication
(10) Pub. No.: US 2024/0279657 A1
(43) Pub. Date: Aug. 22, 2024

(54) COMPOSITIONS AND METHODS FOR THE PREVENTION AND TREATMENT OF HEMOGLOBINOPATHIES

(71) Applicant: Duke University, Durham, NC (US)

(72) Inventors: Rahima ZENNADI, Durham, NC (US); Brittany ELLIOTT, Durham, NC (US); Christopher HOLLEY, Durham, NC (US)

(21) Appl. No.: 18/570,967

(22) PCT Filed: Jun. 16, 2022
(86) PCT No.: PCT/US2022/033859

§ 371 Date: Dec. 15, 2023

Related U.S. Application Data
(60) Provisional application No. 63/211,295, filed on Jun. 16, 2021.

(51) Int. Cl. C12N 15/113; A61K 31/7115; A61P 7/00

(52) U.S. Cl. CPC C12N 15/113; A61K 31/7115; A61P 7/00

(57) ABSTRACT

The present disclosure describes, in part, compositions and method for the prevention and treatment of hemoglobinopathies, including sickle cell disease by inhibition or decreasing production, expression or activity of Rpl13 snoRNAs.

### CROSS-REFERENCE TO RELATED APPLICATIONS

The present application claims priority to U.S. Provisional Patent Application No. 63/211,295, filed Jun. 16, 2021 the entire contents of which are hereby incorporated by reference.

### STATEMENT REGARDING FEDERALLY SPONSORED RESEARCH

This invention was made with government support under R01HL137930 awarded by National Institutes of Health. The government has certain rights in the invention.

### REFERENCE TO A SEQUENCE LISTING

This application is being filed electronically via EFS-Web and includes an electronically submitted Sequence Listing in .txt format. The .txt file contains a sequence listing entitled “155554.00652_ST25” created on Jun. 15, 2022 and is 14,139 bytes in size. The Sequence Listing contained in this .txt file is part of the specification and is hereby incorporated by reference herein in its entirety.

### FIELD OF THE INVENTION

The disclosed technology is generally directed to compositions and methods for treating hemoglobinopathies. More particularly the technology is directed to compositions and methods for treating sickle cell disease through the inhibition of RPL13A snoRNAs.

### BACKGROUND OF THE INVENTION

Small nucleolar RNAs (snoRNAs) are a specific class of small non-protein coding RNAs, that play a major role in regulating gene expression at virtually all levels. These snoRNAs, molecules of 80-200 nucleotides in length, are best known for their function as guide RNAs for posttranscriptional modifications on ribosomal RNA (rRNA) and small nuclear RNA (snRNA). snoRNAs have been divided into two major classes, C/D-box and H/ACA-box snoRNAs, based on sequence and structural features. snoRNAs interact with their specific targets through antisense complementarity and serve to scaffold an RNA modification complex on the target RNA. C/D-box snoRNAs recruit the enzyme fibrillarin to catalyze 2′-O-methylation and H/ACA-box snoRNAs recruit the enzyme dyskerin to catalyze pseudouridylation. As their name implies, snoRNAs are primarily found in the nucleolus, which is the site of rRNA synthesis. However, some snoRNAs reside in the nucleoplasmic Cajal bodies (CBs) where they guide modifications of snRNAs and are called small Cajal body-specific RNAs (scaRNAs). There are >200 well-defined snoRNAs that guide >200 modifications to rRNA and snRNA, and these modifications are thought to contribute to RNA structure in the ribosome and spliceosome, respectively.

The majority of previous research has elucidated the canonical role of snoRNAs in guiding the modification of rRNA and snRNA, but recent studies point to their broader roles in disease pathologies, including Prader-Willi syndrome, dyskeratosis congenita, and some cancers. A class of snoRNAs of the ribosomal protein (Rp) l13a genetic locus has been shown to have a critical non-conical role in regulating metabolic reactive oxygen species (ROS) and oxidative stress in a mouse model of diabetes. These C/D-box Rpl13a snoRNAs (U32a, U33, U34, and U35a) contribute to the development of diabetes, where their loss reduces ROS in islet cells, stimulates insulin secretion, and improves systemic glucose tolerance. Although the exact molecular mechanisms linking Rpl13a snoRNAs to the regulation of ROS and oxidative stress remain somewhat obscure, it has been shown that at least one of these snoRNAs (U32a) can guide 2′-O-methylation on peroxidasin (Pxdn) mRNA, regulating both mRNA and protein expression of this peroxidase. Therefore, although the Rpl13a snoRNAs are known to guide 2′-O-methylation on rRNA, they may be regulating ROS and oxidative stress though alternative modification sites on mRNA.

Oxidative stress due to increased ROS generation is also critical to the pathophysiology of sickle cell disease (SCD). It is hypothesized that Rpl13a snoRNAs are also relevant to SCD pathology. Human SCD is a recessive inherited blood disorder caused by a monogenic A→T mutation of the human β-globin gene [HBB] that results in a substitution of the sixth amino acid of β-globin from β6 GAG→GTG; Glu→Val to produce hemoglobin S (HbS) tetramers. Sickle red blood cells (SS RBCs) are the cause for multiple sources of pro-oxidant processes with consequent links to chronic and systemic oxidative stress. These cells are thus subjected to continuous endogenous and exogenous oxidative onslaughts. It is well established that HbS has the ability to undergo autoxidation to produce ROS within the SS RBC at a rate higher than that produced by normal Hb (HbA). Studies have also evidenced the existence of an enzymatic source, the NADPH oxidase (Nox) enzymes, of excessive ROS production in SS RBCs. As a result of increased oxidative stress, these sickle cells aggregate, interact with other blood cells, and adhere to the vascular endothelium, thus blocking blood vessels, consequently causing severe health complications such as recurrent pain crises, stroke, kidney failure, and heart disease among others. Although mature RBCs lack nuclei, whether SS RBCs retained the cytosolic Rpl13a snoRNAs, and whether these small non-coding RNAs (ncRNAs) remain functional in SS RBCs, and regulate RBC ROS production and ROS-mediated vaso-occlusion, anemia, and organ damage are extremely critical issues in the study of SCD pathophysiology, as due to their mode of action they could become a potential therapeutic target.

### BRIEF SUMMARY OF THE INVENTION

The Summary is provided to introduce a selection of concepts that are further described below in the Detailed Description. This Summary is not intended to identify key or essential features of the claimed subject matter, nor is it intended to be used as an aid in limiting the scope of the claimed subject matter.

The present disclosure is based, in part, on the discovery by the inventor that blocking snoRNAs of the Rpl13a locus, such as U32a, U33, U34 and U35a, can be used to prevent and treat conditions such as vascular occlusion, severe anemia due to intravascular hemolysis, and sickle cell anemia.

Accordingly, one aspect of the present disclosure provides a first composition capable of reducing and/or inhibiting production, expression or activity of a Rp113a snoRNA(s) in a cell and/or subject.

In some embodiments, the snoRNA is selected from the group consisting of U32a, U33, U34, U35a and combinations thereof. The compositions may be antisense oligonucleotides complementary to the snoRNAs and capable of binding to an inhibiting their activity. In some embodiments, the composition comprises SEQ ID NO: 47, SEQ ID NO: 46 and/or SEQ ID NO: 44 alone or combinations thereof.

Another aspect of the present disclosure provides a pharmaceutical composition comprising, consisting of, or consisting essentially of a composition as provided herein and a pharmaceutically acceptable excipient, diluent and/or carrier.

Another aspect of the present disclosure provides a method of reducing and/or inhibiting the production, expression or activity of Rp113a snoRNA(s) in a cell and/or subject, the method comprising, consisting of, or consisting essentially of administering to the cell and/or subject a therapeutically effective amount of a first composition, or a pharmaceutical composition thereof, as provided herein such that the production, expression or activity of the Rp113a snoRNA(s) is reduced and/or inhibited.

Another aspect of the present disclosure provides a method of preventing and/or treating a hemoglobinopathy in a subject, the method comprising, consisting of, or consisting essentially of reducing and/or inhibiting the production, expression, or activity of an Rpl13a snoRNA with a therapeutically effective amount of a first composition, or a pharmaceutical composition thereof, as provided herein such that the expression of the Rp113a snoRNA(s) is reduced and/or inhibited.

In another aspect, the hemoglobinopathy is selected from the group consisting of Thalassemia, Sickle Cell disease, vascular occlusion, beta-thalassemia, severe anemia due to intravascular hemolysis, and combinations thereof. In some embodiments, the composition decreases ROS in sickle cell disease. In some embodiments, the composition increases fetal hemoglobin expression.

In another aspect, the composition comprises a guide RNA, wherein the guide RNA targets at least one Rpl13a snoRNA to allow a knock-out of that snoRNA in a cell. In some embodiments, the guide RNA targets U32a, U34 and/or U35a. Targeting of the snoRNA causes an inhibition in the production of the snoRNA and leads to an alleviation of symptoms of the hemoglobinopathy.

### BRIEF DESCRIPTION OF THE DRAWINGS

FIG. 1A-1E. In vivo RPL13A snoRNA loss lowers sickle RBC H₂O₂ levels. A-B. RPL13A snoRNA expression and H₂O₂ production in human RBCs. Sickle RBCs (SS) from SCD patients (n=9) show high levels of (A) RPL13A snoRNAs U32a, U33, U34 and U35a, and (B) H₂O₂ compared to normal RBCs (AA) from healthy controls (n=9). Relative expression of snoRNAs in SCD patients is normalized to AA patients for each target. C-E. Rpl13a snoRNA genetic knockout in the Townes mice. C. The Townes (hβS/S) mice expressing RPL13A snoRNAs that represent the control SCD (SS/WT) mice, the heterozygous RPL13A snoRNA knockout (SS/Rpl13a snoRNA^{+/-}), and the homozygous RPL13A snoRNA knockout (SS/Rpl13a snoRNA^{-/-}) Townes mice were generated as described in “Material and Methods.” Ribbon Colors that SS/WT, SS/Rpl13a snoRNA^{+/-} and SS/Rpl13a snoRNA^{-/-} mice wear in this figure (red, blue, and yellow, respectively) are used in FIGS. 1-7. D-E. The expression levels of RPL13A snoRNAs U32a, U33, U34 and U35a in RBCs in SS/Rpl13a snoRNA^{+/-} mice (n=6), SS/Rpl13a snoRNA^{-/-} mice (n=14), and the phenotypically normal mice homozygous for WT βA referred to as AA/WT mice (n=6) were significantly lower compared to SS/WT mice (n=14) (D), and these snoRNA levels were associated with RBC ROS levels (8 SS/WT, 6 SS/Rpl13a snoRNA^{+/-}, 6 SS/Rpl13a snoRNA^{-/-}, and 10 AA/WT) (E). Error bars show SEM. **: p<0.01, ***: p<0.001 and ****: p<0.0001 vs. AA RBCs; and ***: p<0.001 and ****: p<0.0001 vs. SS/WT mice.

FIG. 2A-2L. In vivo RPL13A snoRNA depletion in sickle RBCs modulates Nox expression, H₂O₂ production, and peroxidase activity. A-I. RBC Nox expression. NOX1, NOX2 and NOX4 mRNA (A, D, and E) and the respective protein (B, C, F, G, H, and I) expression levels were examined in human sickle (SS; n=12) and normal RBCs (AA; n=12) (A-C), and sickle RBCs from SS/WT (n=22 for mRNA expression; n=10 for protein expression), SS/Rpl13a snoRNA^{+/-} (n=12 for mRNA expression; n=8 for protein expression) and SS/Rpl13a snoRNA^{-/-} (n=10 for mRNA expression; n=8 for protein expression) mice (D-I). NOX1 mRNA abundance is higher in SS vs. AA. Depletion in RPL13A snoRNAs in murine sickle RBCs lowered NOX1 mRNA levels, but increased NOX4 mRNA levels. Membrane protein ghosts (50 μg/lane) were blotted with specific antibodies against Nox1, Nox2, Nox4, and GAPDH as a loading control. Quantitative analysis of Nox1, Nox2 (2 bands) and Nox4 (2 bands) expression normalized according to GAPDH expression are presented. Nox1 expression is up-regulated in SS vs. AA RBCs. RPL13A snoRNA depletion down-regulated Nox1 and Nox2 expression, but up-regulated Nox4 expression in murine sickle RBCs. Data are presented as mean±SEM. **: p<0.01 and ****: p<0.0001 vs. AA; and *: p<0.05, ***: p<0.001 and ****: p<0.0001 vs. SS/WT mice. J-K. RBC H₂O₂ levels and Peroxidase activity. Sickle mice KO for RPL13A snoRNAs (n=11/group of animals) and AA/WT (n=10) mice show significantly higher RBC H₂O₂ levels than those detected in SS/WT mice (J). However, while peroxidase activity is higher in AA (n=9) vs. SS (n=9) RBCs (K), SS/Rpl13a snoRNA^{+/-} (n=17), SS/Rpl13a snoRNA^{-/-} (n=17), and AA/WT mice (n=10) show increased peroxidase activity in murine RBCs compared to sickle RBCs with intact RPL13A snoRNAs (n=14) (L). Plots show mean±SEM. ****: p<0.0001 vs. AA or SS/WT mice.

FIG. 3A-3E. In vivo RPL13A snoRNA depletion in sickle RBCs down-regulates GRK2 and ERK1/2 expression, and ERK1/2 activity. A-B. Kinase mRNA expression levels in RBCs. GRK2, ERK1 and ERK2 mRNA expression levels were examined in human sickle (SS; n=12) and normal RBCs (AA; n=12) (A), and murine sickle RBCs from SS/WT (n=24), SS/Rpl13a snoRNA^{+/-} (n=12) and SS/Rpl13a snoRNA^{-/-} (n=12) mice (B). C-E. GRK2 and ERK1/2 expression, and ERK1/2 phosphorylation in murine sickle RBCs. Membrane protein ghosts (50 μg/per lane) were blotted with specific antibodies against GRK2, ERK1/2, phospho-ERK1/2 (p-ERK1/2; for ERK1/2 activation), and GAPDH as a loading control. Quantitative analysis of GRK2 and ERK1/2, and phospho-ERK1/2 normalized according to GAPDH expression are presented. Sickle RBC GRK2 and ERK1/2 expression, and ERK1/2 activity are down-regulated following RPL13A snoRNA depletion. Data are presented as mean±SEM. **: p<0.01 vs. AA; and *: p<0.05 and ****: p<0.0001 vs. SS/WT mice.

FIG. 4.A-4C RPL13A snoRNA knockout in sickle mice lowers tissue organ ROS production and improves organ damage. A-C. Tissue organs, the kidneys (A), liver (B), and spleen (C) were used to determine ROS levels in the different sickle mouse genotypes. Heterozygous and homozygous knockouts of U32a, U33, U34 and U35a in the kidneys, liver, and spleen in sickle mice decreased ROS levels compared to SS/WT mice (n=6 per group of animals). Error bars show SEM. **: p<0.01, and ****: p<0.0001 vs. SS/WT mice.

FIG. 5A-5J. RPL13A snoRNA knockout in sickle mice improves organ damage. A-G. Organ tissue pathology. A. Low (200×) and high (600×) magnification photomicrographs of H&E staining of the kidney from SS/WT and SS/Rpl13a snoRNA^{-/-} mice. Note vascular, tubule-interstitial, and glomerular changes within proximal tubular epithelium of the outer cortex in SS/WT mice, and glomerulopathy in SS/Rpl13a snoRNA^{-/-} mice as indicated with arrows. B. Low magnification (200×) photomicrographs of Prussian blue iron staining of kidney outer cortex from SS/WT, SS/Rpl13a snoRNA^{+/-} and SS/Rpl13a snoRNA^{-/-} mice. Note lack of blue iron positive tubules in the kidney sections of SS/Rpl13a snoRNA^{+/-} and SS/Rpl13a snoRNA^{-/-} mice. C. Low (200×) magnification photomicrographs of H&E staining of the liver from SS/WT and SS/Rpl13a snoRNA^{+/-} and SS/Rpl13a snoRNA^{-/-} mice. The photomicrographs depicting multifocal necrosis and extensive associated inflammatory and reactive changes in SS/WT mice as indicated with arrows, but normal hepatic parenchyma devoid of necrosis in SS/Rpl13a snoRNA^{+/-} and SS/Rpl13a snoRNA^{-/-} mice. D. Low (200×) magnification photomicrographs of Prussian blue staining of the liver from SS/WT and SS/Rpl13a snoRNA^{+/-} and SS/Rpl13a snoRNA^{-/-} mice showing iron blue positive deposits and brown pigment containing macrophages and Kupffer cells in SS/WT mice, but absent iron deposits in the liver of SS/Rpl13a snoRNA^{+/-} and SS/Rpl13a snoRNA^{-/-} mice. E. RPL13A snoRNA loss improves splenomegaly (n=8/group). Error bars show SEM; ****: p<0.0001 vs. SS/WT mice. F-G. Low (200×) magnification photomicrographs of the spleen from SS/WT and SS/Rpl13a snoRNA^{+/-} mice. SS/Rpl13a snoRNA^{+/-} mice lack significant splenic ectasia within red pulp areas (H&E staining; F), and accumulation of brown pigment (Prussian blue staining; G) compared with SS/WT mice. H-J. Organ cell apoptosis. Representative labeling studies with GFP-labeled annexin V of cells isolated from organs: kidneys, liver, and spleen harvested from SS/WT (n=9), SS/Rpl13a snoRNA^{+/-} (n=9) and SS/Rpl13a snoRNA^{-/-} (n=9) mice. RPL13A snoRNA knockout reduces organ damage as reflected by decreased early-stage cell apoptosis compared to the SS/WT group. Data are presented as the mean±SEM; *: p<0.05, ***: p<0.001, and ****: p<0.0001 vs. SS/WT mice.

FIG. 6A-6I. RPL13A snoRNA knockout in sickle mice ameliorates leukocytosis and anemia. A-I. Leukocytosis (A-D) and anemia (E-I) severity evaluated in sickle mice following RPL13A snoRNA depletion. Blood drawn through submandibular vein of SS/WT (n=8), SS/Rpl13a snoRNA^{+/-} (n=8), SS/Rpl13a snoRNA^{-/-} (n=8), AA/WT (n=10), WT/Rpl13a snoRNA^{-/-} (n=8), and WT/WT (n=8) mice was assessed for complete blood count. Total leukocyte (A), neutrophil (B), lymphocyte (C), monocyte (D) and RBC (E) counts, hemoglobin (F), hematocrit (G), MCHC (H), and reticulocyte percentages (I) are shown. Values represent means±SEM. **: p<0.01 and ****: p<0.0001 vs. SS/WT mice.

FIG. 7A-7F. RPL13A snoRNA loss reduces cell adhesion and vaso-occlusion in sickle mice and ameliorates survival rate. A. Schematic representation of intravital microscopy protocol. Anesthetized sickle mice with dorsal skin-fold window chamber implants were injected with rhodamine 6G or PE-conjugated anti-mouse TER119 antibody to label leukocytes and RBCs, respectively, as described in “Material and Methods.” Thirty minutes later, mice were challenged with TNF-α (time 0). After 120 minutes, intravital microscopy was performed and blood cell behavior in the subdermal vasculature was recorded between the time points of 120 and 180 minutes (T₁₂₀→T₁₈₀). B. Representative images of post-capillary venules (20× magnification) from SS/WT (n=6), SS/Rpl13a snoRNA^{+/-} (n=6) and SS/Rpl13a snoRNA^{-/-} (n=6) mice were presented. Vessels without adherent cells appear gray, due to the rapidly moving of fluorescence-labeled cells. Sickle RBC and leukocyte adhesion is indicated with arrows. Scale bar=50 μm. C-E. Video frames were used to quantify fluorescence-labeled Leukocyte rolling across a specific point per minute presented as # cells/min (C), adhesion of fluorescence-labeled leukocytes and RBCs presented as # of adherent cells/100 μm vessel length (D), and blood flow presented as percentage of vessels with normal, slow and no blood flow (E). Vessels analyzed were almost similar in diameter (˜25 μm) among all groups tested. Error bars show SEM. **: p<0.01, ***: p<0.001, and ****: p<0.0001 compared to SS/WT regardless of the vessel diameter. F. The Kaplan-Meier survival curves for SS/WT, SS/Rpl13a snoRNA^{+/-} and SS/Rpl13a snoRNA^{-/-} mice (n=11 per group). Log-rank (Mantel-Cox) test, p<0.0001.

FIG. 8A-8B. Mouse genotyping. A. Primers (straight arrows) and predicted size (nucleotides) of genotyping PCR products. B. Representative agarose gel of PCR products from mouse tail DNA is shown (n=12).

FIG. 9A-9C. Depletion in Rpl13a snoRNA expression in organs in sickle mice. Tissue organs, the kidneys, liver, and spleen were used to determine Rpl13a snoRNA in sickle mouse mutants. A-C. Quantification of Rpl13a snoRNA U32a, U33, U34, and U35a abundance in the kidneys (A), liver (B), and spleen (C) of SS/WT (n=12), SS/Rpl13a snoRNA^{+/-} (n=6) and SS/Rpl13a snoRNA^{-/-} (n=6) mice. The abundance of each Rpl13a snoRNA was normalized to the control Rplp0. Error bars show SEM. **: p<0.01, ***: p<0.001, and ****: p<0.0001 vs. SS/WT mice.

FIG. 10A-10E. In vivo Rpl13a snoRNA depletion lowers sickle RBC ROS and H₂O₂ levels. a-b. Rpl13a snoRNA expression and H₂O₂ production in human RBCs. Human sickle (SS) RBCs from SCD patients (n=17) show high levels of (a) Rpl13a snoRNAs U32a, U33, U34 and U35a, and (b) H₂O₂ compared to normal (AA) RBCs from healthy controls (n=17). c-e. Genetic knockouts of Rpl13a snoRNAs in the Townes mice. The Townes (hβS/S) mice expressing Rpl13a snoRNAs that represent the control SCD (SS/WT) mice, and the Townes mice Rpl13a snoRNA-knockout (SS/Rpl13a snoRNA-null) were generated as described in “Material and Methods.” Knockout of Rpl13a snoRNAs in sickle mice significantly lowers the expression levels of (c) U32a, U33, U34, and U35a in sickle RBCs (n=8), compared to SS/WT mice (n=8), and this effect was accompanied by reduced levels of sickle RBC (d) ROS and (e) increased H₂O₂. Error bars show SEM. ***: p<0.001 and ****: p<0.0001 vs. AA RBCs or SS/WT mice; and **: p<0.01 vs. SS/WT mice.

FIG. 11A-11E. Rpl13a snoRNA knockout in sickle mice and K562 cell line modulates RBC Nox4 expression. a-c. Murine sickle RBC Nox4 expression. mRNA (a) and protein (b-c) expression levels of Nox4 were examined in murine sickle RBCs from SS/WT (n=9), and SS/Rpl13a snoRNA-null (n=9) mice. a. Loss of Rpl13a snoRNAs in murine sickle RBCs increased Nox4 mRNA levels. b. Cell lysates (50 μg/lane) were blotted with specific antibodies against Nox4, and GAPDH as a loading control. c. Quantitative analysis of Nox4 expression normalized according to GAPDH expression are presented. Rpl13a snoRNA depletion up-regulated Nox4 protein expression in murine sickle RBCs. d-e. Nox4 expression and ROS production in K562 cell lines. K562 cell lines with CRISPR/Cas9 knockout (KO) for U32a, U33, U34, U35a, and the control U25 were assessed for the levels of Nox4 mRNA expression and ROS production. Each K562 KO cell line is an independent biological sample, analyzed in three independent experiments. d. While depleting the Rpl13a snoRNA U34 in K562 U34 KO cell line increased Nox4 mRNA expression, loss of the Box C/D snoRNAs U33, and U35a in K562 U33 KO, and K562 U35a KO cell lines, respectively, decreased Nox4 mRNA levels. e. Knocking-out U33, U34, or U35a in K562 cells reduced ROS. Data are presented as mean±SEM. ***: p<0.001 and ****: p<0.0001 vs. SS/WT mice; and *: p<0.05 and **: p<0.01 vs. the control K562 U25 KO.

FIG. 12A-12H. Box C/D snoRNAs U33, U34, and U35a in human SS RBCs and K562 cells regulate Nox4 mRNA abundance by assembling into complexes with HuR. a-f. Cell lysates were prepared from (a-d) human SS RBCs (n=3), and (e-h) K562 cell lines with CRISPR/Cas9 KO for U33, U34, U35a, and the control U25. Each K562 KO cell line is an independent biological sample, analyzed in three independent experiments. Immunoprecipitation was performed with antibody to HuR. Parallel immunoprecipitations with normal rabbit IgG1 served as negative controls. Panels a-b and e-f show Western analysis of the immunoprecipitated HuR (a and e) run alongside non-precipitated lysate (b and f) from (a-b) human SS RBCs, and (e-f) K562, and K562 U33 KO, U34 KO, U35a KO, and U25 KO cell lines. Panels c, d, g, and h report RT-qPCR quantification of co-precipitating Rpl13a snoRNAs (c, and g) and Nox4 mRNA (d, and h) from (c-d) human SS RBCs and (g-h) K562 cell lines as a ratio of RNA recovered with the specific antibody relative to IgG1.

FIG. 13A-13D. Peroxidase activity, PRDX2 mRNA 2′-O-methylation, and PRDX2 mRNA and protein expression levels are altered in human SS RBCs. RBCs from SCD patients and from healthy volunteers were analyzed. a. Peroxidase activity. Peroxidase activity in Human SS and AA RBCs was measured using Amplex Red. Peroxidase activity in lower in SS RBCs (n=9) vs. AA RBCs (n=9). b-d. Compared to AA RBCs, SS RBCs have higher (b) PRDX2 mRNA 2′-O-methylation and (c) PRDX2 mRNA abundance, but (d) lower PRDX2 protein levels determined by immunoblotting with normalization to total protein per lane. Plots show mean±SEM. *: p<0.05, **: p<0.01 and ****: p<0.0001 vs. AA RBCs.

FIG. 14A-14D. Murine erythroid Rpl13a snoRNAs guide PRDX2 mRNA 2′-O-methylation to regulate PRDX2 mRNA translation, and thus protein levels in vivo. a-d. Sickle RBCs from SS/WT, and SS/Rpl13a snoRNA-null mice were analyzed. Deficiency in Rpl13a snoRNAs in sickle RBCs from SS/Rpl13a snoRNA-null mice reduced (a) PRDX2 mRNA 2′-O-methylation and (b) PRDX2 mRNA abundance, but increased (c) PRDX2 protein levels and (d) peroxidase activity compared to SS/WT mice. Plots show mean±SEM. *: p<0.05, ***: p<0.001 and ****: p<0.0001 vs. SS/WT mice.

FIG. 15A-15D. PRDX2 mRNA 2′-O-methylation requires box C/D snoRNAs U32a and U34. a-d. The wild type K562 cells, and K562 cell lines with CRISPR/Cas9 KO for U32a (n=4), U33 (n=4), U34 (n=4), U35a (n=4), and U25 (n=4) were tested for (a) PRDX2 mRNA 2′-O-methylation, (b) PRDX2 mRNA expression levels, (c) PRDX2 protein expression levels, and (d) peroxidase activity. Each K562 KO cell line is an independent biological sample, analyzed in four independent experiments. Knocking-out the C/D box snoRNA U32a or U34 in K562 cell lines lowered (a) PRDX2 mRNA 2′-O-methylation and (b) PRDX2 mRNA abundance, but increased (c) PRDX2 protein expression and (d) peroxidase activity compared to the wild type and/or to the K562 U25 KO cells. Data are presented as mean±SEM. *: p<0.05, **: p<0.01 and ****: p<0.0001 vs. the control K562 U25 KO.

FIG. 16A-16G. In vivo knocking-down the box C/D snoRNAs U34 and U35a reduces cell adhesion and vaso-occlusion triggered by TNF-α in sickle mice. a-g. Rpl13a snoRNA genetic knockdown in the Townes mice. Sickle mice were injected with GFP-ASO to knockdown (KD) sequence from GFP (GFP KD control; n=6), U34-ASO and U35a-ASO to double-KD U34 and U35a, respectively (U34+U35a KD; n=6), or U32a-ASO, U34-ASO and U35a-ASO to triple-KD U32a, U34, and U35a, respectively (U32a+U34+U35a KD; n=6) as described in “Material and Methods.” a-b. Significant in vivo KD of the box C/D snoRNAs U34 and U35a, and the box C/D snoRNAs U32a, U34 and U35a in U34+U35a KD and U32a+U34+U35a KD sickle mice, respectively, lowers ROS levels vs. the GFP KD controls. Error bars show SEM. **: p<0.01, ***: p<0.001 and ****: p<0.0001 compared to the GFP KD group. c. Schematic representation of intravital microscopy protocol. The protocol was designed to assess the effect of Rpl13a snoRNA loss on reducing vaso-occlusion triggered by TNF-α in sickle mice. Anesthetized sickle mice with dorsal skin-fold window chamber implants were injected with rhodamine 6G and PE-conjugated anti-mouse TER119 mAb to label leukocytes and RBCs, respectively. Thirty minutes later, mice were challenged with TNF-α (time 0). 120 minutes post-TNF-α injection, intravital microscopy was performed and blood cell behavior in the subdermal vasculature was recorded between the time points of 120 and 180 minutes (T₁₂₀→T₁₈₀). d. Representative images of post-capillary venules (20× magnification) from sickle mice injected with GFP-ASO, U34-ASO and U35a-ASO, or U32a-ASO, U34-ASO and U35a-ASO were presented. Vessels without adherent cells appear gray, due to the rapidly moving of fluorescence-labeled cells. Murine sickle RBC and leukocyte adhesion is indicated with arrows. Scale bar=50 μm. Video frames were used to quantify adhesion of fluorescence-labeled leukocytes and RBCs presented as # of adherent cells/100 μm vessel length (e), and blood flow presented as percentage of vessels with normal, slow and no flow (f). Vessels analyzed were almost similar in diameter (˜25 μm) among all groups tested. Error bars show SEM. ****: p<0.0001 compared to the GFP KD control group regardless of the vessel diameter. g. RBCs from GFP KD sickle mice not injected with TNF-α, and U34+U35a KD and U32a+U34+U35a KD sickle mice treated with TNF-α, were tested for ROS levels. Error bars show SEM. *: p<0.05 vs. GFP KD control not injected with TNF-α.

FIG. 17. Assessment of K562 snoRNA KO cell lines. CRISPR/Cas9 methods were used in K562 cells to knockout snoRNAs U32a, U33, U34, U35a, and U25. RT-qPCR demonstrated complete and specific loss of the targeted snoRNAs U32a, U33, U34, or U35a compared to parental wild type K562 or the K562 U25 KO controls. Each K562 cell line is an independent biological sample, analyzed in four independent experiments. Mean and SEM error bars are shown.

FIG. 18A-18D. Rpl13a snoRNA knockdown in organs in sickle mice. a-d. Relative quantification of expression of box C/D snoRNAs U34 and U35a, and box C/D snoRNAs U32a, U34 and U35a in (a) the kidneys, (b) liver, (c) spleen, and (d) lungs in GFP KD (n=4), U34+U35a KD (n=6), and U32a+U34+U35a KD (n=6) sickle mice are presented as mean±SEM. *: p<0.05 and **: p<0.01 vs. GFP KD controls.

FIG. 19A-19D. Knocking-down Rpl13a snoRNAs U34 and U35a, or U32a, U34 and U35a in sickle mice lowers tissue organ ROS production. a-d. Tissue organs, the kidneys (a), liver (b), spleen (c), and lungs (d) were used to determine tissue ROS levels in the different sickle mouse genotypes. Knockdown of U34 and U35a, or U32a, U34 and U35a in sickle mice decreased ROS levels in the kidneys, liver, spleen, and lungs compared to the controls GFP-KD animals (n=6 per group of animals). Error bars show SEM. *: p<0.05, **: p<0.01, and ****: p<0.0001 vs. GFP KD controls.

FIG. 20A-20C. Rpl13a snoRNAs loss in sickle mice reduces sickling, increases F-cell numbers, and down-regulates erythroblast transcription factor mRNA levels involved in silencing γ-globin gene expression. a. Sickling presented as % sickled RBCs following cell exposure to 1% 02(99% N₂) was improved as a result of Rpl13a snoRNAs loss. The data presented as SEM of n=6 per group; ****: p<0.0001 vs. SS/WT mice. b. Rpl13a snoRNA loss increases % circulating F-cells. The data presented as SEM of n=6 per group; *: p<0.0001 vs. SS/WT mice. c. mRNA levels of the transcription factors BCL11A, KLF1, SOX6 and ATF4, and SIRT1 deacetylase in erythroblast generated from the bone marrow of SS/WT, SS/Rpl13a snoRNA^{+/-}, and SS/Rpl13a snoRNA^{-/-} mice. Data are presented as fold change in mRNA levels. p<0.05 over SS/WT mice.

### DETAILED DESCRIPTION OF THE INVENTION

### A. Compositions

Aspects of the present disclosure provide for compositions that are capable of reducing and/or inhibiting anRpl13a snoRNA(s). Accordingly, one aspect of the present disclosure provides a first composition capable of reducing and/or inhibiting the production, expression, or activity of a Rp113a snoRNA(s) in a cell and/or subject.

As used herein, “reducing” means an amount below, or less than the amount prior to treatment. As used herein, “inhibiting” means to control, prevent, restrain, arrest, or regulate the action, function or expression of snoRNAs. In some embodiments a composition described herein may reduce or inhibit the expression of Rpl13a snoRNA such that the expression of Rpl13a snoRNA is less following the administration of the composition. The expression or activity of a snoRNA can be reduced or inhibited via binding to a complementary antisense oligonucleotide.

Small nucleolar RNAs (snoRNAs) are a class of small RNA molecules that primarily guide chemical modifications of other RNAs, mainly ribosomal RNAs, transfer RNAs and small nuclear RNAs. There are two main classes of snoRNA, the C/D box snoRNAs, which are associated with methylation, and the H/ACA box snoRNAs, which are associated with pseudouridylation. SnoRNAs are also referred to as guide RNAs. Ribosomal protein L13a (Rpl13a) encodes a member of the L13P family of ribosomal proteins and is a component of the 60S ribosomal subunit. Mammalian loci for rpL13a contain four highly conserved intronic box C/D small nucleolar RNAs (snoRNAs) that are predicted to be processed during splicing of the rpL13a pre-mRNA transcript. These snoRNAs termed U32a, U33, U34, and U35a, are located within the introns of Rpl13a.

snoRNA U32a, U33, U34 and U35a are also known as SNORD32a, SNORD33, SNORD34 and SNORD35a respectively and are located in the nucleolus of a eukaryotic cell. These snoRNAs are a C/D box class of snoRNAs which contain the conserved sequence motifs known as the C box (UGAUGA) and the D box (CUGA). The box C/D snoRNAs are primarily known to guide post-transcriptional modifications, especially 2′-O-methylation, of ribosomal RNA and small nuclear RNA. U32a may comprise the sequence of SEQ ID NO: 49 in humans or SEQ ID NO: 52 in mice, U34 may comprise the sequence of SEQ ID NO: 51 in humans or SEQ ID NO: 54 in mice, U35a may comprise the sequence of SEQ ID NO: 56 in humans and SEQ ID NO: 55 in mice.

In some embodiments, Rpl13a snoRNA(s) are decreased. Rpl13a snoRNA may be decreased by any means known in the art. These include, but are not limited to, RNA-based RNA interference including siRNA, and shRNA, DNA-based RNA interference, including antisense oligonucleotides, non-homologous end joining, and CRISPR-mediated gene knockdown or knockout, including using dCas9 with or without addition proteins, Cas12a and Cas13 family enzymes, full or partial gene deletion or gene editing or mutation, non-homologous end joining or Transcription Activator-Like Effector Nucleases (TALENs). In some embodiments, an antisense oligonucleotide (ASO) is used to reduce or inhibit the activity of the snoRNA. ASOs are short, synthetic, chemically modified chains of nucleotides that have the potential to target any gene or nucleotide product of interest. Typically, an ASO is a single-stranded sequence complementary to the sequence of the target's messenger RNA (mRNA) within a cell. The ASO used herein, may be complementary to Rpl13a snoRNA, including U32a, U33, U34 or U35a (presented as SEQ ID NOs: 49-51 and 56 for humans and SEQ ID NOs: 52-55 for the mouse, respectively). An ASO complementary to a single Rpl13a snoRNA may be used or ASO which target multiple Rpl13a snoRNA. The sequence of the ASO for U32a may comprise SEQ ID NO: 44, the sequence of the ASO for U34 may comprise SEQ ID NO: 47, the sequence of the ASO for U35a may comprise SEQ ID NO: 46. These ASOs are representative and one skilled in the art could modify the ASO to target the snoRNA. These ASOs used in the Examples were 20 nucleotides in length and contained 2′-O-methoxyribonucleotide segments of 5 nucleotides at both termini and a deoxynucleotide segment containing 10 central nucleotides. All of the phosphate backbones were converted by phosphorothioate. These modifications were made to the ASO to increase stability. Those skilled in the art are aware of other modifications that may be made to an ASO to modify the immune reaction to the ASO or increase its stability or pharmacokinetics. The ASOs may be DNA, RNA or mixed RNA/DNA hybrids. The aptamers may include a polynucleotide (RNA, DNA, or locked nucleic acids (LNA) or peptide nucleic acid (PNA)) that is in an unmodified form or may be in a modified form including at least one nucleotide base modification. Nucleotide base modifications of polynucleotides to, for example, protect the polynucleotide from nuclease degradation and/or increase the stability of the polynucleotide and are well-known in the art. Common nucleotide base modifications that may be used in accordance with the present invention include, without limitation, deoxyribonucleotides, 2′-O-Methyl bases, 2′-Fluoro bases, 2′ Amino bases, inverted deoxythymidine bases, 5′ modifications, and 3′ modifications. In some embodiments, the aptamer may include a polynucleotide including a modified form including at least one nucleotide base modification selected from the group consisting of a 2′fluoro modification, a 2′O-methyl modification, a 5′ modification, and a 3′ modification. Typical 5′ modifications may include, without limitation, inverted deoxythymidine bases, addition of a linker sequence such as C6, addition of a cholesterol, addition of a reactive linker sequence which could be conjugated to another moiety such as a PEG. Typical 3′ modifications may include, without limitation, inverted deoxythymidine bases, and inverted abasic residues. The ASO used in the examples were 20 nucleotides in length and the full length was complementary to the targeted snoRNA, but the ASO can be as short as 15 nucleotides, or as long as the full length of the snoRNA or any length in between. The ASO need not be fully complementary to the snoRNA and can tolerate some mismatches especially if a longer ASO is used.

Administration of ASO can be carried out using the various mechanisms known in the art, including naked administration and administration in pharmaceutically acceptable carriers. For example lipid carriers may be used. In general, ASO may be delivered via injection into a subject or cell to decrease expression of Rpl13a snoRNA. The injection may be an intraperitoneal injection (IP), intravenous, intramuscular, subcutaneous or intradermal. The ASO may be delivered via oral routes, transfection, electroporation, microinjection, gene gun or magnetic-assisted transfection.

The amount of ASO administered is one effective to inhibit the expression of RPl13a snoRNA. It will be appreciated that this amount will vary both with the effectiveness of the ASO delivered, the route of delivery and the nature of the carrier used. The determination of appropriate amounts for any given composition is within the skill in the art. The ASO may be delivered daily, every other day, or at another regular interval, for 1, 2, 3, 4, 5, 6, 7 or more days. Individual ASO may be delivered or multiple ASO may be combined and delivered together, for example SEQ ID NO: 47 and SEQ ID NO: 46 or SEQ ID NO: 47 and SEQ ID NO: 46 and SEQ ID NO: 47 may be administered together.

### B. Pharmaceutical Compositions

In another aspect, the present disclosure provides pharmaceutical compositions comprising one or more of the compositions as described herein and an appropriate carrier, excipient or diluent. The exact nature of the carrier, excipient or diluent will depend upon the desired use for the composition and may range from being suitable or acceptable for veterinary uses to being suitable or acceptable for human use. The composition may optionally include one or more additional compounds.

When used to treat or prevent a disease or symptoms of a disease, such as vascular occlusion, severe anemia due to intravascular hemolysis, and sickle cell anemia, the compositions described herein may be administered singly, as mixtures of one or more compounds or in mixture or combination with other agents (e.g., therapeutic agents) useful for treating such diseases and/or the symptoms associated with such diseases. Such agents may include, but are not limited to, hydroxyurea, L-glutamine oral powder, Crizanlizumab, pain-relieving medications (e.g., NSAIDs, etc.), Voxelotor, nitroglycerin, steroids (e.g., prednisone), sildenafil, iron supplementation, to name a few. The compounds may be administered in the form of compounds per se, or as pharmaceutical compositions comprising a compound.

Pharmaceutical compositions comprising the compound(s) may be manufactured by means of conventional mixing, dissolving, granulating, dragee-making levigating, emulsifying, encapsulating, entrapping or lyophilization processes. The compositions may be formulated in conventional manner using one or more physiologically acceptable carriers, diluents, excipients or auxiliaries which facilitate processing of the compounds into preparations which can be used pharmaceutically.

Pharmaceutical compositions may take a form suitable for virtually any mode of administration, including, for example, topical, ocular, oral, buccal, systemic, nasal, injection, transdermal, rectal, vaginal, etc., or a form suitable for administration by inhalation or insufflation.

For topical administration, the compound(s) may be formulated as solutions, gels, ointments, creams, suspensions, etc. as are well-known in the art. Systemic formulations include those designed for administration by injection, e.g., subcutaneous, intravenous, intramuscular, intrathecal or intraperitoneal injection, as well as those designed for transdermal, transmucosal oral or pulmonary administration.

Useful injectable preparations include sterile suspensions, solutions or emulsions of the active compound(s) in aqueous or oily vehicles. The compositions may also contain formulating agents, such as suspending, stabilizing and/or dispersing agent. The formulations for injection may be presented in unit dosage form, e.g., in ampules or in multidose containers, and may contain added preservatives. Alternatively, the injectable formulation may be provided in powder form for reconstitution with a suitable vehicle, including but not limited to sterile pyrogen free water, buffer, dextrose solution, etc., before use. To this end, the active compound(s) may be dried by any art-known technique, such as lyophilization, and reconstituted prior to use.

For transmucosal administration, penetrants appropriate to the barrier to be permeated are used in the formulation. Such penetrants are known in the art.

For oral administration, the pharmaceutical compositions may take the form of, for example, lozenges, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art with, for example, sugars, films or enteric coatings.

Liquid preparations for oral administration may take the form of, for example, elixirs, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol, Cremophore™ or fractionated vegetable oils); and preservatives (e.g., methyl or propyl-p-hydroxybenzoates or sorbic acid). The preparations may also contain buffer salts, preservatives, flavoring, coloring and sweetening agents as appropriate.

Preparations for oral administration may be suitably formulated to give controlled release of the compound, as is well known. For buccal administration, the compositions may take the form of tablets or lozenges formulated in conventional manner. For rectal and vaginal routes of administration, the compound(s) may be formulated as solutions (for retention enemas) suppositories or ointments containing conventional suppository bases such as cocoa butter or other glycerides.

For nasal administration or administration by inhalation or insufflation, the compound(s) can be conveniently delivered in the form of an aerosol spray from pressurized packs or a nebulizer with the use of a suitable propellant, e.g., dichlorodifluoromethane, trichlorofluoromethane, dichlorotetrafluoroethane, fluorocarbons, carbon dioxide or other suitable gas. In the case of a pressurized aerosol, the dosage unit may be determined by providing a valve to deliver a metered amount. Capsules and cartridges for use in an inhaler or insufflator (for example capsules and cartridges comprised of gelatin) may be formulated containing a powder mix of the compound and a suitable powder base such as lactose or starch.

For ocular administration, the compound(s) may be formulated as a solution, emulsion, suspension, etc. suitable for administration to the eye. A variety of vehicles suitable for administering compounds to the eye are known in the art.

For prolonged delivery, the compound(s) can be formulated as a depot preparation for administration by implantation or intramuscular injection. The compound(s) may be formulated with suitable polymeric or hydrophobic materials (e.g., as an emulsion in an acceptable oil) or ion exchange resins, or as sparingly soluble derivatives, e.g., as a sparingly soluble salt.

Alternatively, transdermal delivery systems manufactured as an adhesive disc or patch which slowly releases the compound(s) for percutaneous absorption may be used. To this end, permeation enhancers may be used to facilitate transdermal penetration of the compound(s). Alternatively, other pharmaceutical delivery systems may be employed. Liposomes and emulsions are well-known examples of delivery vehicles that may be used to deliver compound(s). Certain organic solvents such as dimethyl sulfoxide (DMSO) may also be employed, although usually at the cost of greater toxicity.

The pharmaceutical compositions may, if desired, be presented in a pack or dispenser device which may contain one or more unit dosage forms containing the compound(s). The pack may, for example, comprise metal or plastic foil, such as a blister pack. The pack or dispenser device may be accompanied by instructions for administration.

The compositions described herein will generally be used in an amount effective to achieve the intended result, for example in an amount effective to treat or prevent the particular disease being treated. By therapeutic benefit is meant eradication or amelioration of the underlying disorder being treated and/or eradication or amelioration of one or more of the symptoms associated with the underlying disorder such that the patient reports an improvement in feeling or condition, notwithstanding that the patient may still be afflicted with the underlying disorder. Therapeutic benefit also generally includes halting or slowing the progression of the disease, regardless of whether improvement is realized.

The amount of composition administered will depend upon a variety of factors, including, for example, the particular indication being treated, the mode of administration, whether the desired benefit is prophylactic or therapeutic, the severity of the indication being treated and the age and weight of the patient, the bioavailability of the particular composition, the conversion rate and efficiency of delivery under the selected route of administration, etc.

Determination of an effective dosage for a particular use and mode of administration is well within the capabilities of those skilled in the art. Effective dosages may be estimated initially from in vitro activity and metabolism assays. For example, an initial dosage for use in animals may be formulated to achieve a circulating blood or serum concentration of the composition that is at or above an IC₅₀ of the particular composition as measured in an in vitro assay. Calculating dosages to achieve such circulating blood or serum concentrations taking into account the bioavailability of the particular composition via the desired route of administration is well within the capabilities of skilled artisans. Initial dosages can also be estimated from in vivo data, such as animal models. Animal models useful for testing the efficacy of the active metabolites to treat or prevent the various diseases described above are well-known in the art. Animal models suitable for testing the bioavailability and/or metabolism of compositions are also well-known. Ordinarily skilled artisans can routinely adapt such information to determine dosages suitable for human administration.

Dosage amounts will typically be in the range of from about 0.0001 mg/kg/day, 0.001 mg/kg/day or 0.01 mg/kg/day to about 100 mg/kg/day, but may be higher or lower, depending upon, among other factors, the activity of the active composition, the bioavailability of the composition, its metabolism kinetics and other pharmacokinetic properties, the mode of administration and various other factors, discussed above. Dosage amount and interval may be adjusted individually to provide plasma levels which are sufficient to maintain therapeutic or prophylactic effect. For example, the compositions may be administered once per week, several times per week (e.g., every other day), once per day or multiple times per day, depending upon, among other things, the mode of administration, the specific indication being treated and the judgment of the prescribing physician. In cases of local administration or selective uptake, such as local topical administration, the effective local concentration of compositions may not be related to plasma concentration. Skilled artisans will be able to optimize effective dosages without undue experimentation.

### C. Methods

Another aspect of the present disclosure provides a method of reducing and/or inhibiting the production, expression or activity of Rp113a snoRNA(s) in a cell and/or subject, the method comprising, consisting of, or consisting essentially of administering to the cell and/or subject a therapeutically effective amount of a first composition, or a pharmaceutical composition thereof, as provided herein such that the expression of the Rp113a snoRNA(s) is reduced and/or inhibited in a subject or cell.

Another aspect of the present disclosure provides a method of reducing and/or inhibiting the function or activity of a snoRNA associated with Rp113a in a cell and/or subject, the method comprising, consisting of, or consisting essentially of administering to the cell and/or subject a therapeutically effective amount of a second composition, or a pharmaceutical composition thereof, as provided herein such that the function or activity of the snoRNA is reduced and/or inhibited.

Methods of the present disclosure further provide for the prevention and/or treatment of hemoglobinopathies in a subject by administration of compositions that inhibit the activity of a snoRNA associated with RPl13. The methods of preventing and/or treating a hemoglobinopathy in a subject comprise, consist of, or consist essentially of reducing and/or inhibiting the expression or activity of a snoRNA that is associated with Rp113a. These methods may include administering the compositions and pharmaceutical compositions provided herein. In some embodiments, the hemoglobinopathy is selected from the group consisting of Thalassemia, Sickle Cell disease, vascular occlusion, severe anemia due to intravascular hemolysis, and combinations thereof.

One aspect of the present disclosure provides a method of reducing and/or inhibiting reactive oxygen species (ROS), the method comprising, consisting of, or consisting essentially of administering to the cell and/or subject a therapeutically effective amount of a composition provided herein such that ROS is decreased. Reactive oxygen species (ROS) are highly reactive chemicals formed from oxygen (O₂). Examples of ROS include peroxides, superoxide, hydroxyl radical, singlet oxygen, and alpha-oxygen. The reduction of molecular oxygen (O₂) produces superoxide (·O₂⁻), which is the precursor to most other reactive oxygen species. ROS are byproducts of the normal metabolism of oxygen.

In the present disclosure, ROS may be produced in conjunction with hemoglobinopathies, including sickle cell disease. ROS may be decreased and/or inhibited by the decrease and/or inhibition of Rpl13a snoRNA. ROS may be decreased and/or inhibited by the decrease and/or inhibition of Rpl13a snoRNA U32a, U34 and/or U35 alone or in combination.

The methods provided herein may further comprise a guide RNA, wherein the guide RNA targets RPl13a including a RPl13a snoRNA. A guide RNA (gRNA) is a piece of RNA that functions as a guide for RNA- or DNA-targeting enzymes, with which it forms complexes. Very often these enzymes will delete, insert or otherwise alter the targeted RNA or DNA using the CRISPR-Cas system. The guide RNA is a specific RNA sequence that recognizes the target DNA or RNA region of interest and directs the Cas nuclease for editing. The gRNA may be made up of two parts: crispr RNA (crRNA), a 17-20 nucleotide sequence complementary to the target DNA or RNA, and a tracr RNA, which serves as a binding scaffold for the Cas nuclease or these two may be combined into a single guide RNA (sgRNA). In the present disclosure, the gRNA may target U32a, U34 and/or U35a. Guide RNA may be used individually or in combination as needed to reduce and/or inhibit Rpl13a snoRNA. Thus, CRISPR-Cas gene editing may be used to knock-out one or more snoRNAs associated with Rpl13a, such as U32a, U33, U34 or U35a.

One aspect of the present disclosure provides methods and compositions for increasing fetal hemoglobin. Fetal hemoglobin may also be called foetal haemoglobin, hemoglobin F, HbF, or α_{2γ2}. HbF is made in the fetus and newborn, then gradually decreases and reaches adult levels (less than 1% of total hemoglobin) usually within the first year. HbF has 4 heme groups, allowing it to bind to up to four oxygen molecules, composed of two α (alpha) subunits and two γ (gamma) subunits. Reactivating γ-globin gene of fetal hemoglobin can alleviate some symptoms of sickle cell disease. Increased HbF may be measured, among other means, by change in sickling of red blood cells, percent of F-cells, change in transcription factors including KLF1, BCL11A, SOX6 and/or ATF4, increased red blood cell counts, Hb, hematocrit, mean corpuscular Hb (MCHC), or reticulocyte counts. By way of example, but not by way of limitation, the disclosed methods may be utilized to increase fetal hemoglobin in a subject.

Another aspect of the present disclosure provides all that is described and illustrated herein.

### Miscellaneous

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term. “About” is used to provide flexibility to a numerical range endpoint by providing that a given value may be “slightly above” or “slightly below” the endpoint without affecting the desired result.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition. As used herein, the terms “prevent,” “preventing,” “prevention,” “prophylactic treatment” and the like refer to reducing the probability of developing a disease, disorder or condition in a subject, who does not have, but is at risk of or susceptible to developing a disease, disorder or condition. The term “effective amount” or “therapeutically effective amount” refers to an amount sufficient to effect beneficial or desirable biological and/or clinical results.

As used herein, the term “administering” an agent, such as a therapeutic entity to an animal or cell, is intended to refer to dispensing, delivering or applying the substance to the intended target. In terms of the therapeutic agent, the term “administering” is intended to refer to contacting or dispensing, delivering or applying the therapeutic agent to a subject by any suitable route for delivery of the therapeutic agent to the desired location in the animal, including delivery by either the parenteral or oral route, intramuscular injection, subcutaneous/intradermal injection, intravenous injection, intrathecal administration, buccal administration, transdermal delivery, topical administration, and administration by the intranasal or respiratory tract route.

The term “biological sample” as used herein includes, but is not limited to, a sample containing tissues, cells, and/or biological fluids isolated from a subject. Examples of biological samples include, but are not limited to, tissues, cells, biopsies, blood, lymph, serum, plasma, urine, saliva, mucus and tears. In one embodiment, the biological sample is a biopsy (such as a tumor biopsy). A biological sample may be obtained directly from a subject (e.g., by blood or tissue sampling) or from a third party (e.g., received from an intermediary, such as a healthcare provider or lab technician).

The term “disease” as used herein includes, but is not limited to, any abnormal condition and/or disorder of a structure or a function that affects a part of an organism. It may be caused by an external factor, such as an infectious disease, or by internal dysfunctions, such as cancer, cancer metastasis, genetic mutations, and the like. In some embodiments, a disease comprises disease and/or disorder and/or condition that is characterized by the Rpl13a locus and associated snoRNAs, such as U32a, U33, U34 and U35a. In one embodiment, the disease, disorder and/or condition comprises a hemoglobinopathy. Examples of such diseases, disorders and/or conditions include, but are not limited to, vascular occlusion, severe anemia due to intravascular hemolysis, beta-thalassemia, and sickle cell anemia.

“Contacting” as used herein, e.g., as in “contacting a sample” refers to contacting a sample directly or indirectly in vitro, ex vivo, or in vivo (i.e. within a subject as defined herein). Contacting a sample may include addition of a compound to a sample, or administration to a subject. Contacting encompasses administration to a solution, cell, tissue, mammal, subject, patient, or human. Further, contacting a cell includes adding an agent to a cell culture.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Unless otherwise defined, all technical terms used herein have the same meaning as commonly understood by one of ordinary skill in the art to which this disclosure belongs.

### EXAMPLES

### Example I—RPL13a Small Nucleolar RNA Depletion Ameliorates Oxidative Stress-Induced Sickle Cell Pathophysiology In Vivo

### Introduction

Small nucleolar RNAs (snoRNAs) are non-coding RNAs primarily known to guide post-transcriptional modifications on ribosomal RNA (rRNA) and small nuclear RNA (snRNA)^{1, 2}. SnoRNAs are typically 80-200 nucleotides in length, and they are divided into “C/D-box” and “H/ACA-box” classes based on sequence and structural features³⁻⁵. SnoRNAs interact with their specific targets through antisense complementarity and serve to scaffold an RNA modification complex on the target RNA. C/D-box snoRNAs recruit the enzyme fibrillarin to catalyze 2′-O-methylation and H/ACA-box snoRNAs recruit the enzyme dyskerin to catalyze pseudouridylation⁶⁻¹⁰. These small non-coding RNAs (snRNAs) are primarily found in the nucleolus, which is the site of rRNA synthesis. There are >200 well-defined snoRNAs that guide >200 modifications to rRNA and snRNA, and these modifications are thought to contribute to RNA structure in the ribosome and spliceosome, respectively.

In addition to this canonical role guiding the modification of rRNA and snRNA, it has been evidenced that snoRNAs of the Ribosomal protein L13a (Rpl13a) locus are fundamental regulators of metabolic reactive oxygen species (ROS) and oxidative stress¹¹. These C/D-box RPL13A snoRNAs (U32a, U33, U34, and U35a) contribute to the development of diabetes, where their loss reduces ROS in islet cells, stimulates insulin secretion, and improves systemic glucose tolerance¹². The presence of the RPL13A snoRNAs in the cytosol have suggested a non-canonical targets for these snoRNAs outside the nuclear structure.

Oxidative stress is critical to the pathophysiology of sickle cell disease (SCD)¹³⁻¹⁵. The SS RBC is the primary source of excessive ROS production and oxidative stress in SCD¹⁶, and additionally to hemoglobin S (HbS) autoxidation¹⁶, NADPH oxidases (Nox) activation contributes to increased ROS generation within SS RBCs^{17, 18}. As a result of oxidative stress, these sickle cells aggregate, interact with other blood cells, and adhere to the vascular endothelium, thus blocking blood vessels¹⁸⁻²² and causing severe health complications such as recurrent pain crises, stroke, kidney failure, and heart disease²³⁻²⁸. Given the central importance of oxidative stress in SCD, and the fundamental regulation of ROS levels and oxidative stress by RPL13A snoRNAs, we sought to define the pathological relevance of RPL13A snoRNAs in SCD. Here we show that Rpl13a snoRNAs play a critical role in regulating the pathophysiology of SCD, and that reducing the levels of these ncRNAs is effective in a mouse model of the disease.

### Material and Methods

Antibodies. Monoclonal and polyclonal antibodies used were purified immunoglobulin [Ig], and they were against the following human or mouse proteins: glyceraldehyde 3-phosphate dehydrogenase (GAPDH) (Santa Cruz biotechnology, Dallas, TX); Nox1 (Novus Biological, Centennial, CO); Nox2 (Abcam, Cambridge, MA); Nox4 (Abcam); G protein-coupled receptor kinase 2 (GRK2) (Santa Cruz Biotechnology); anti-mitogen-activated protein kinase (MAPK) ERK1/2 (Upstate, Charlottesville, VA); and anti-phospho-ERK1/2 (Cell Signaling Technology, Danvers, MA). In all studies, Abs were used at saturating dilutions.

RBC preparation. Blood samples collected from human participants has been approved by Duke University's Institutional Review Board and written informed consent has been obtained from the participants. Blood samples were collected from adult SCD patients homozygous for HbS, and from healthy donors. SCD patients had not been transfused for at least three months and had not experienced acute vaso-occlusive crises for three weeks, and 98% of the patients tested were on hydroxyurea. Blood samples were collected into citrate tubes. RBCs were washed extensively in PBS with removal of the plasma and buffy coat. Packed RBCs were analyzed for leukocyte and platelet contaminations using an Automated Hematology Analyzer K-1000 (Sysmex Corporation, Kobe, Japan).

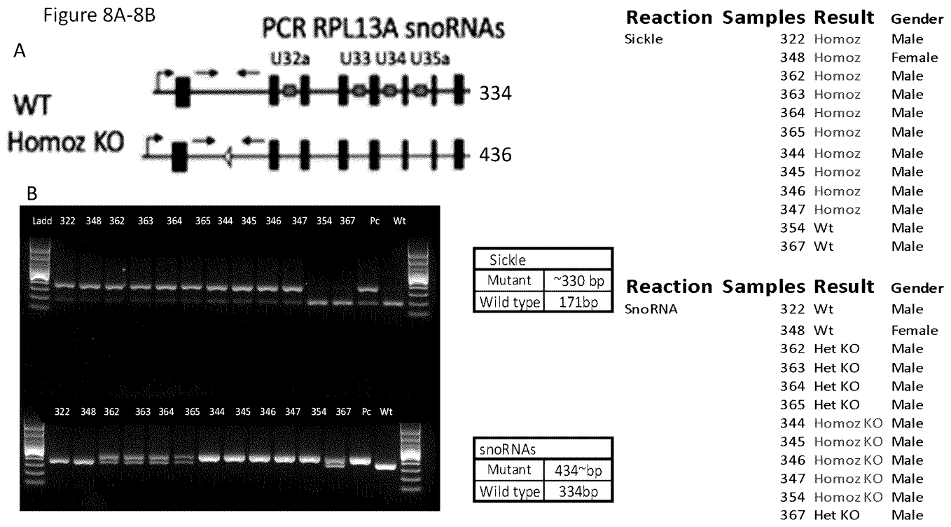

Mice. The Institutional Animal Care and Use Committee (IACUC) and the Committee on the Ethics of Animal Experiments at Duke University approved this animal work. The transgenic sickle mice, the Townes mice B6; 129-Hba^{tm1(HBA)Tow}/Hbb^{tm2(HBG1,HBB*)Tow}/Hbb^{tm3(HBG1,HBB)Tow}/J, were established by Dr. T. Townes at the University of Alabama^{29, 30}. Townes mice express exclusively human α- and sickle β-globin, which have pathologic findings similar to SCD patients^{31, 32}. The RPL13A snoRNA knockout (KO) mice referred to as wild-type (WT)/Rpl13a snoRNA^{-/-} mice, were generated by the Schaffer Laboratory at Washington University in St. Louis¹². Sickle mice expressing wild-type (WT) RPL13A snoRNAs, and sickle mice heterozygous and homozygous RPL13A snoRNAs KO referred here as SS/WT, SS/Rpl13a snoRNA^{+/-} and SS/Rpl13a snoRNA^{-/-} mice, respectively, were generated by Zennadi Lab at Duke University by crossing sickle mice with RPL13A snoRNA KO mice. All mice used in our experiments were homozygous for sickle alleles (SS), and genotyping was confirmed by PCR (FIG. 8). The phenotypically normal mice homozygous for WT RA referred to as AA/WT, WT/Rpl13a snoRNA^{-/-} and the normal C57BL/6J mice referred to as WT/WT were used as controls in some experiments. Animals, female, and male were used at 10-14 weeks of age.

ROS and peroxidase activity detection. ROS and H₂O₂ production were measured using CM-H2-DCFDA (DCF; Invitrogen, Carlsbad, CA) for ROS detection, and Amplex Red H₂O₂/Peroxidase Assay Kit (Molecular Probes, Grand Island, NY) for H₂O₂ detection following the manufacturer's instructions and as described previously in detail. One hundred thousand events per sample were acquired and tested by flow cytometric analysis. Peroxidase activity was detected using Amplex Red H₂O₂/Peroxidase Assay Kit following the manufacturer's instructions.

Quantitative real-time PCR (RT-qPCR). Total RNA was isolated from RBCs, and from organs using TRIzol reagent (Ambion) according to the manufacturer. cDNAs were prepared using SuperScript® III First-Strand Synthesis kit (Invitrogen). Relative mRNA expression in RBCs of RPL13a snoRNAs U32a, U33, U34 and U35a, NOX1, NOX2, NOX4, GRK2, ERK1, and ERK2, and in organs of RPL13a snoRNAs U32a, U33, U34 and U35a were measured, then normalized to an endogenous control 36B4 (Rplp0) gene. Sequences of primers used to assess gene expression are described below.

Western blot. RBCs were lysed with hypotonic buffer (5 mM Na₂HPO₄+1 mM EDTA+0.1% NaN₃, pH 8) containing 2 mM phenylmethylsulphonyl fluoride (PMSF), phosphatase inhibitor cocktail (Sigma-Aldrich) and protease inhibitor cocktail (Sigma-Aldrich). Protein separation by polyacrylamide gel electrophoresis used 50 μg RBC membrane ghost proteins per lane. Proteins were also separated by polyacrylamide gel electrophoresis from homogenized tissue samples, using 20 μg proteins per lane. Western blots using the appropriate Ab were performed. Bands were analyzed densitometrically using ImageJ software downloaded from the NIH website. Protein expression and phosphorylation data were normalized according to GAPDH expression.

Tissue apoptosis. Prior to organ harvest, animals were weighed. Organs were then harvested from sacrificed animals. Spleen was weighed, and spleen weight/gram body weight was determined. Single cell suspensions were prepared from the different organs, then stained with GFP-conjugated Annexin V (BioLegend) for apoptosis detection as recommended by the manufacturer. Cells were washed, then one hundred thousand events per sample were acquired and analyzed by flow cytometry.

Histology. Harvested organs were fixed in Mildform 10N (Wako Pure Chemical Industries). Tissues were embedded in paraffin, sectioned at 5 microns, and stained with hematoxylin and eosin (H&E), and Perl's Prussian Blue iron stain for light microscopy.

Complete blood count analysis. Complete blood count (CBC) was determined in peripheral blood collected from non-sedated SS/WT, SS/Rpl13a snoRNA^{+/-}, SS/Rpl13a snoRNA^{-/-}, AA/WT, WT/Rpl13a snoRNA^{-/-}, and WT/WT mice. CBC was performed by automated determination of the absolute numbers and ratios of various cell types using Abaxis CBC machine model VetScan Hm5C (Abaxis Inc., Union City, CA).

Reticulocyte counts. Blood samples collected from non-sedated SS/WT, SS/Rpl13a snoRNA^{+/-}, SS/Rpl13a snoRNA^{-/-}, AA/WT, WT/Rpl13a snoRNA^{-/-}, and WT/WT mice were incubated with 10 mg/ml thiazole orange for 30 minutes at room temperature in the dark. Data acquisitions were then performed using flow cytometric analysis.

Window chamber surgery. Following anesthesia, all dorsal skin-fold window chamber surgery was carried out on anesthetized mice under sterile conditions with aseptic technique using a laminar flow hood. Animals were placed on a heated pad at 37° C. General anesthesia was achieved with isoflurane (Butler Animal Health Supply, Dublin, OH), by adjusting the oxygen flowmeter to approximately 0.5-1.0 L/min, and isoflurane vaporizer to 3% for induction and 1% for maintenance. The back of anesthetized mouse was alternatively wiped with surgical sponges soaked in Hibiclens and then alcohol. Hair on the clean area was shaved, and the rest of hair was removed using hair removal cream. The area was cleaned again with Hibiclens and then alcohol. A window chamber consisting of a double-sided titanium frame was surgically implanted into the dorsal skin fold. Surgery involved careful removal of the epidermal and dermal layers of one side of a dorsal skin flap, exposing blood vessels of the subcutaneous tissue adjacent to the striated muscles of the opposing skin fold, and then securing the two sides of the chamber to the skin using stainless steel screws and sutures. Sutures were used to secure the window chamber, which will be retained for the duration of the experiment to maintain the window chambers. A glass window was placed in the chamber to cover the exposed tissue and secured with a snap ring.

Intravital microscopy. Anesthetized animals were injected through a tail vein with 100 μL (0.02% in sterile saline) rhodamine 6G (Sigma-Aldrich, St. Louis, MO) and 0.25 μg/g body weight PE-conjugated anti-mouse TER119 (Ly-76) monoclonal antibody (mAb; Biolegend, San Diego, CA) for in vivo labeling and monitoring of leukocytes and RBCs, respectively. After 30 minutes, mice were injected intra-peritoneal (IP) with 500 ng murine recombinant tumor necrosis alpha (TNFα) to precipitate vaso-occlusion. Ninety minutes later, intravital microscopy was performed. At least 20 venules of each mouse were recorded.

Adhesion of leukocytes and RBCs was quantified by counting the number of adherent cells (stationary cells for longer than 30 seconds) along the length of a given venule, and expressed as average number of cells per 100 μm length of the vessel, by analysis of frame-by-frame of video replay using Icy bioimage analysis software (http://icy.bioimageanalysis.org/). Rolling flux was determined by counting the number of leukocytes rolling through a given point in a vessel (#leukocytes/min) by analyzing frame-by-frame video replay using Icy bioimage analysis software. The values obtained from analyzing all recorded vessel segments, were averaged among groups of animals for statistical analysis. The percentages of vessels with normal blood flow, slow blood flow, and occluded vessels were calculated by dividing the number of each: vessels (both small and large) with normal blood flow, slow blood flow, and no blood flow by the total number of vessels recorded in all animals.

Statistical analysis. Data were compared using parametric analyses (GraphPad Prism 9 Software, San Diego, CA), including repeated and non-repeated measures of analysis of variance (ANOVA). One-way ANOVA analyses were followed by Bonferroni corrections for multiple comparisons (multiplying the p value by the number of comparisons). A p value<0.05 was considered significant.

### Results

RPL13A snoRNAs Regulate SS RBC ROS Generation and Oxidative Stress.

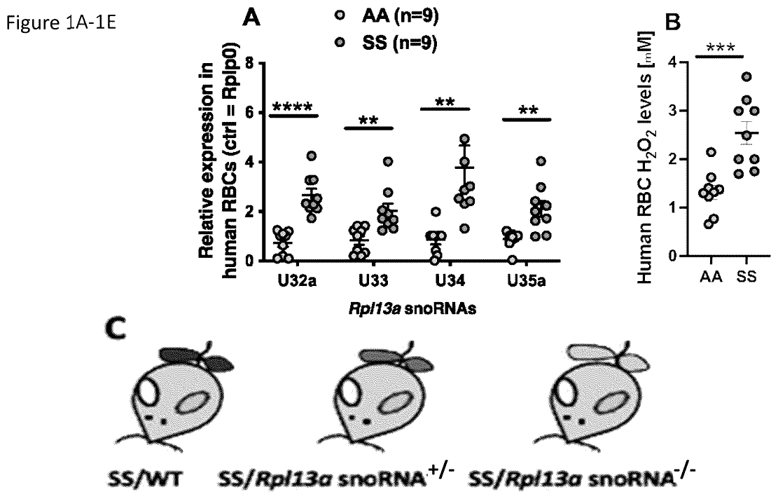

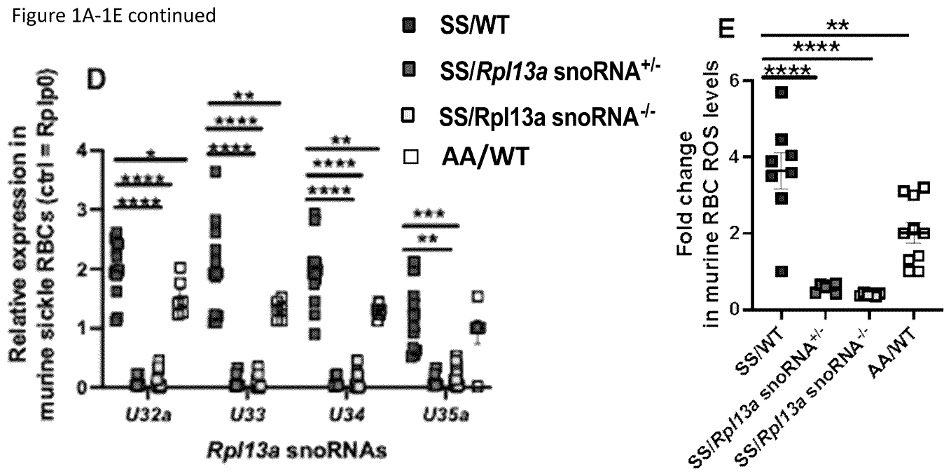

We first analyzed RBCs collected from healthy controls (n=9) and patients with SCD (n=9) for the presence of RPL13A snoRNAs. Quantification of total RPL13A snoRNAs U32A, U33, U34, and U35A by RT-qPCR showed that RPL13A snoRNAs are present in human normal (AA) and SS RBCs (FIG. 1A), showing that snoRNAs are present in RBCs. Notably, human SS RBCs have consistently higher levels of RPL13A snoRNAs U32A, U33, U34, and U35A (p<0.01; FIG. 1A), with higher H₂O₂ levels than AA RBCs (p<0.05; FIG. 1B).

To evaluate whether RPL13A snoRNAs regulate ROS generation in sickle RBCs, we used SS/WT, SS/Rpl13a snoRNA^{+/-} and SS/Rpl13a snoRNA^{-/-} mice (FIG. 1C). We initially compared the health of SS/Rpl13a snoRNA^{+/-} and ^{-/-} animals to the wild-type mice in which the mouse beta-globin locus has been replaced with an insert for human beta-globin (AA/WT). On the SS background, Rpl13a snoRNA^{+/-} and ^{-/-} animals remained for more than two years as healthy as the phenotypically normal AA/WT mice, until the mice were euthanized. As expected and similar to AA/WT mice (p<0.01), RBC U32a, U33, U34 and U35a expression was reduced or eliminated in both SS/Rpl13a snoRNA^{+/-} (p<0.01) and ^{-/-} (p<0.001) mice (FIG. 1D), and ROS levels were lowered by 84% and 89%, respectively, compared to SS/WT mice (p<0.0001; FIG. 1E), suggesting that RPL13A snoRNAs are necessary for ROS generation to levels seen in human SS RBCs.

RPL13A snoRNAs Regulate Sickle RBC Nox Expression, and Peroxidase Activity.

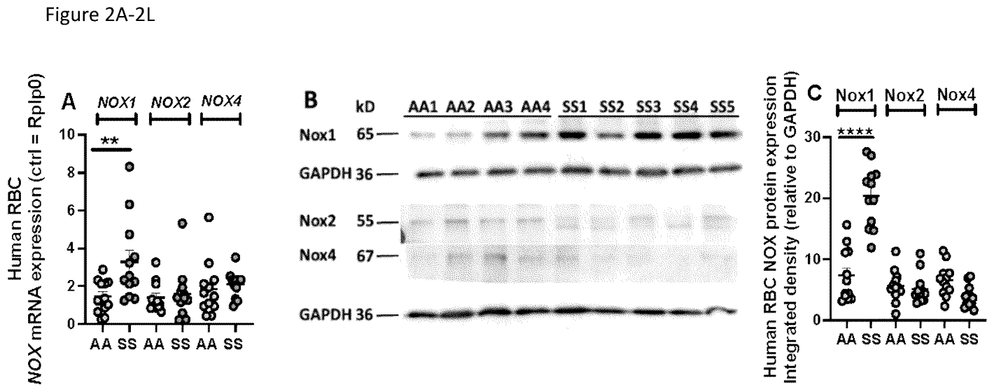

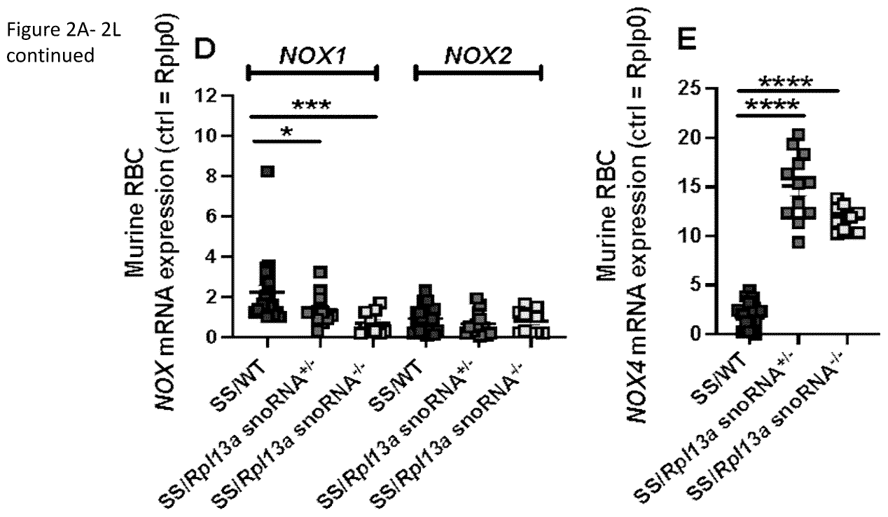

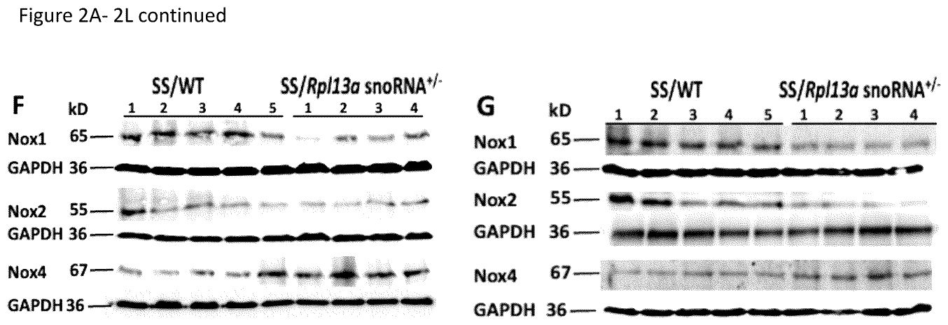

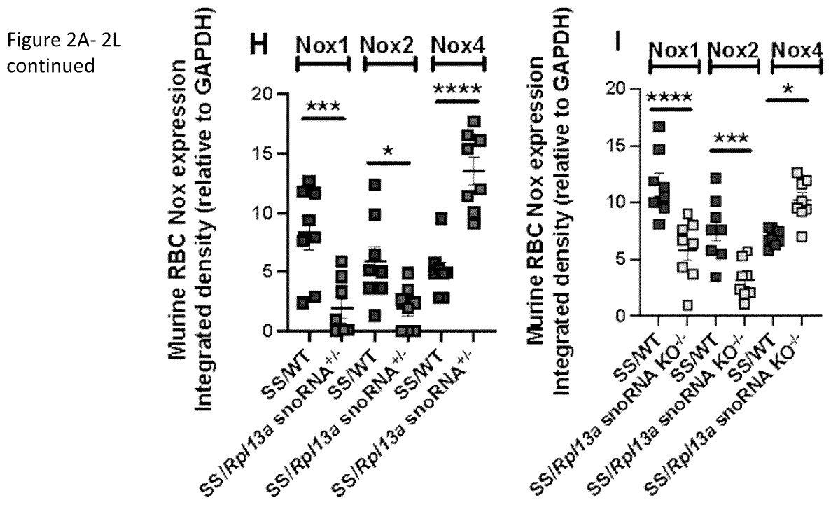

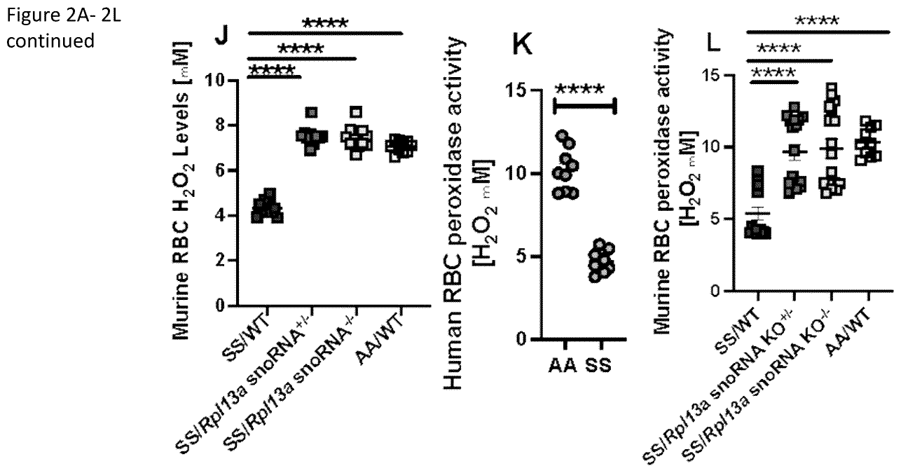

Excessive ROS generation in human and murine sickle RBCs involves Nox activation^{18, 33}. Here, we evaluated mRNA expression of NOX1, NOX2, and NOX4, and their respective proteins in human SS compared to AA RBCs¹⁷. Human SS RBCs had significantly higher levels of NOX1 mRNA than AA RBCs (n=12 per group; p=0.0033), but NOX2 and NOX4 mRNA levels were similar in both cell populations (FIG. 2A). Protein expression paralleled the mRNA levels (FIG. 2B-C). These data suggest that in SCD patients, increased SS RBC ROS production may be due at least partly to higher Nox1 expression/activity relative to AA RBCs.

We then determined the impact of in vivo RPL13A snoRNA loss in murine sickle RBCs on Nox1, Nox2, and Nox4 expression. In sickle mice, heterozygous and homozygous loss of RPL13A snoRNAs significantly decreased sickle RBC NOX1 mRNA levels (p<0.05; FIG. 2D), but drastically increased NOX4 mRNA levels (p<0.0001; FIG. 2E). Nox1 and Nox4 protein expression levels mirrored the respective mRNA levels (p<0.05; FIG. 2F-I). While sickle RBC NOX2 mRNA expression levels were not affected by RPL13A snoRNA depletion (FIG. 2D), protein expression levels were significantly lowered in these cells (p<0.05; FIG. 2F-I).

Nox1 and Nox2 Generate Primarily Superoxide, which Explains the Observed Reduced ROS Levels (FIG. 1E) Following Rpl13a snoRNA Loss in Sickle RBCs, Whereas the Major Detected Product of Nox4, which is Constitutively Active, is H₂O₂.

We thus determined the levels of H₂O₂ in murine sickle RBCs depleted and not depleted in RPL13A snoRNAs, and the activity of peroxidases in both human and in murine sickle RBCs also depleted and not depleted in RPL13A snoRNAs. Peroxidases consume H₂O₂, transferring electrons to acceptor proteins or generating other forms of molecular ROS that have much shorter half-lives than H₂O₂, such that peroxidase activity can lower the steady-state pool of both ROS and H₂O₂. Similar to AA/WT mice, sickle RBC H₂O₂ levels were much higher in SS/Rpl13a snoRNA^{+/-} and SS/Rpl13a snoRNA^{-/-} mice vs. SS/WT mice (p<0.0001; FIG. 2J). However, peroxidase activity in human SS RBCs was significantly lower than in AA RBCs (p<0.01; FIG. 2K), and depleting RPL13A snoRNAs in sickle mice increased significantly murine sickle RBC peroxidase activity to levels comparable to those detected in RBCs in AA/WT mice (p<0.0001; FIG. 2L). These data strongly suggest that RPL13A snoRNAs regulate mRNA expression levels, with consequences for protein expression and physiologic enzyme activity.

RPL13A snoRNAs Modulate Sickle RBC GRK2 and ERK1/2 Expression.

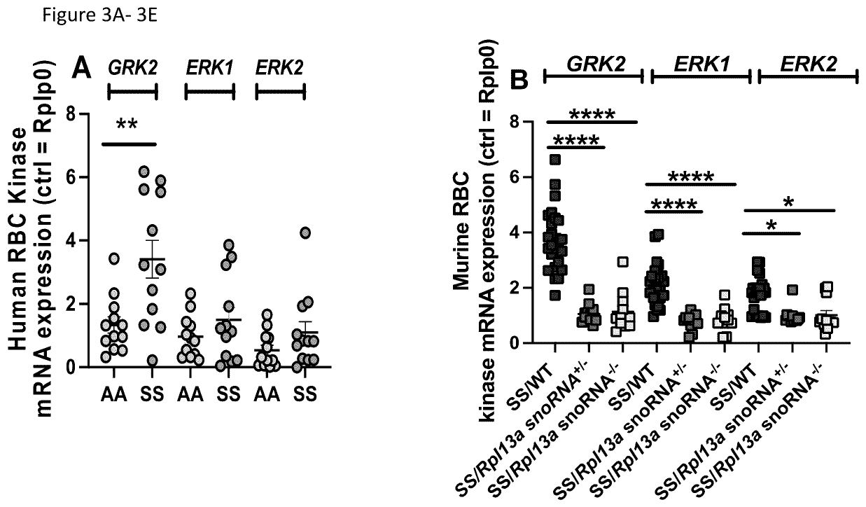

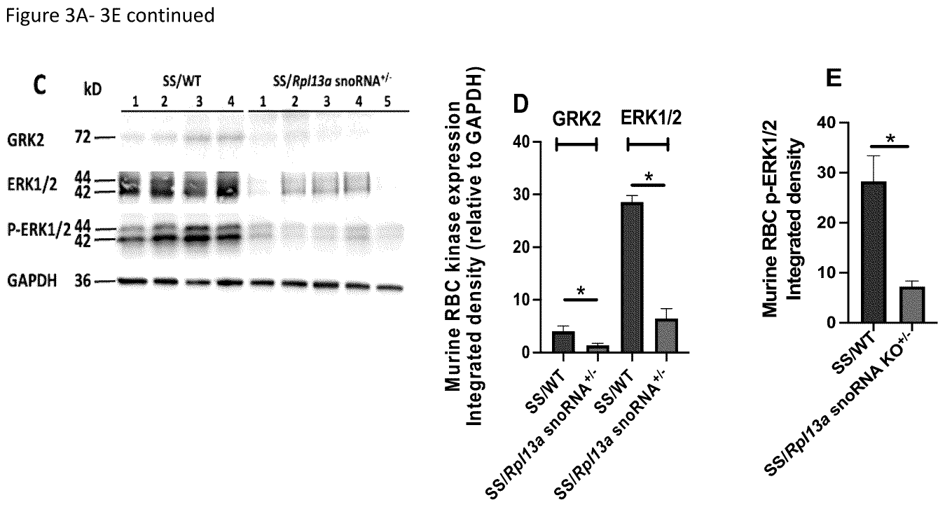

GRK2 and ERK1/2 in human SS, but not in AA, RBCs create a positive feedback loop with Nox-dependent ROS production^{18, 35}. We further defined the relationship between RPL13A snoRNAs and the expression of GRK2 and ERK1/2. We show that GRK2 mRNA expression was significantly higher in human SS RBCs compared to AA RBCs (p=0.004; FIG. 3A), which paralleled our previously published GRK2 expression levels and activity¹⁸. ERK1 and ERK2 mRNA expression levels, however, were similar in SS and AA RBCs (p>0.05; FIG. 3A). Deleting RPL13A snoRNAs in either SS/Rpl13a snoRNA^{+/-} or SS/Rpl13a snoRNA^{+/-} animals diminished sickle RBC Grk2, Erk1, and Erk2 mRNA expression levels (p<0.05; FIG. 3B). The down-regulated kinase mRNA levels due to heterozygous RPL13A snoRNA depletion were strongly associated with reduced expression of the respective proteins (p<0.05; FIG. 3C-D). This was also reflected in ERK1/2 activity, as assessed by phospho-ERK1/2 (p<0.05; FIGS. 3C and 3E). Our findings suggest that RPL13A snoRNAs regulate the positive ROS feedback loop involving GRK2 and ERK1 2 in the context of SCD, and that loss/reduction in RPL13A snoRNAs reduces the pathologic kinase expression and activity.

RPL13A snoRNA Depletion Improves Tissue Organ Oxidative Stress.

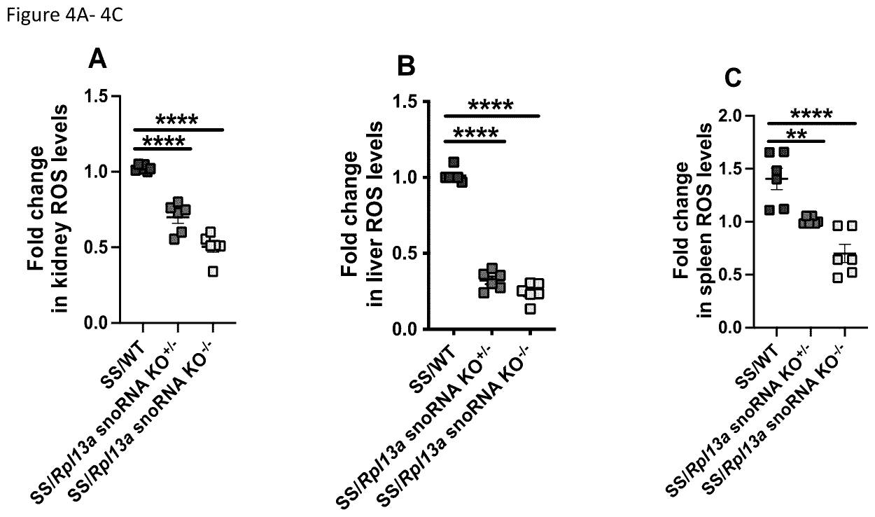

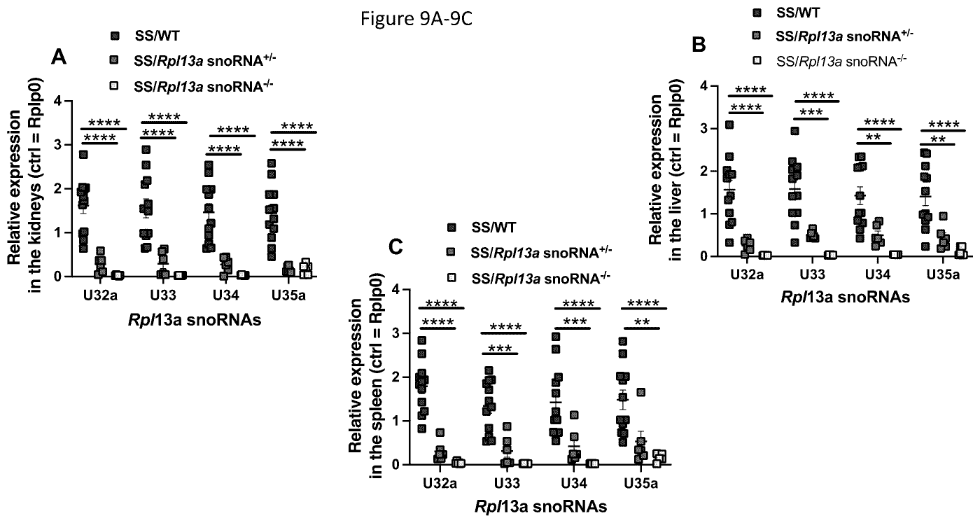

Oxidative stress is an important systemic feature of SCD and plays a key role in ensuing end-organ damage, where the kidneys, liver, and spleen are typically affected in patients^{36, 37}. We examined whether RPL13A snoRNAs also regulate end-organ ROS levels in sickle mice. First, we confirmed that expression levels of U32a, U33, U34 and U35a were significantly reduced or lost in SS/Rpl13a snoRNA^{+/-} and SS/Rpl13a snoRNA^{-/-} mice compared to SS/WT mice, for the kidney (p<0.0001), liver (p<0.01), and spleen (p<0.01) (FIG. 9A-C). As expected, heterozygous and homozygous RPL13A snoRNA loss decreased end-organ ROS compared to SS/WT mice (p<0.01 for each; FIG. 4A-C), suggesting that RPL13A snoRNAs regulate end-organ ROS production and oxidative stress in sickle mice, which could contribute to the systemic pathology of SCD in these animals.

RPL13A snoRNA Depletion Ameliorates End-Organ Injury in Sickle Mice.

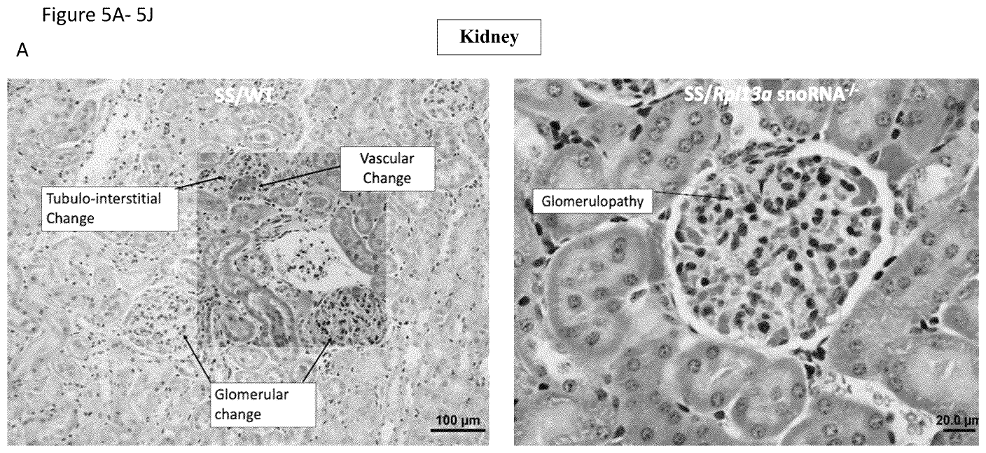

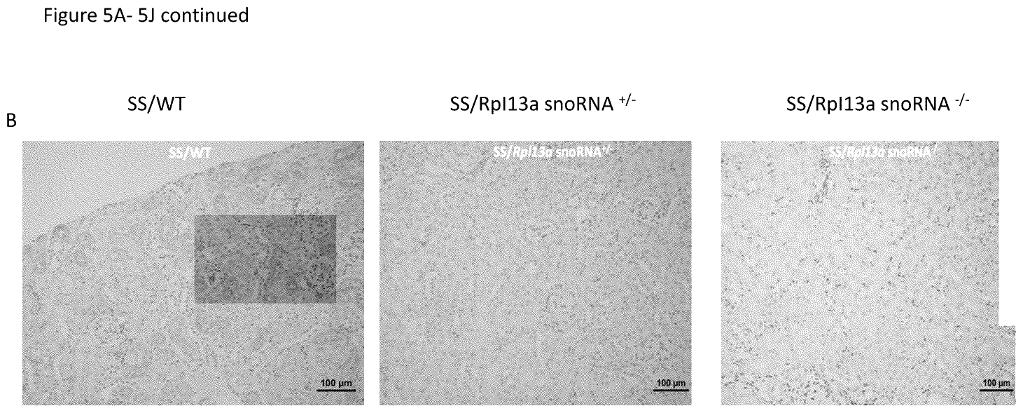

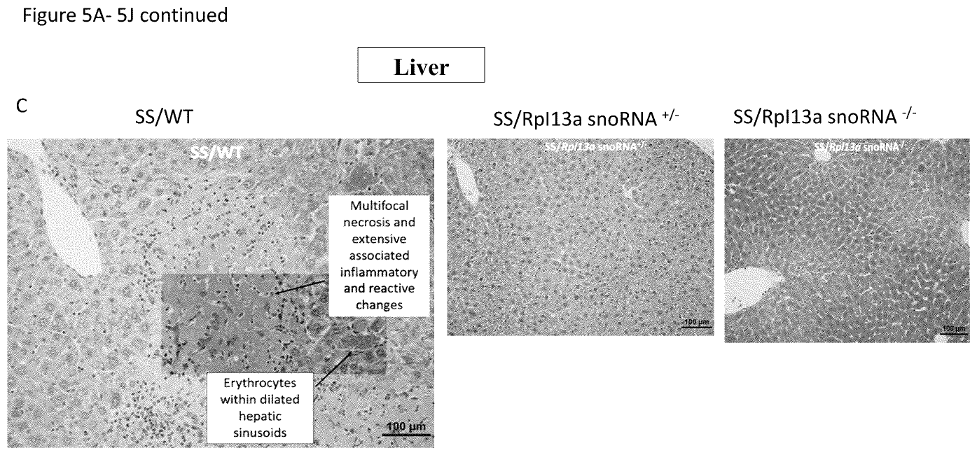

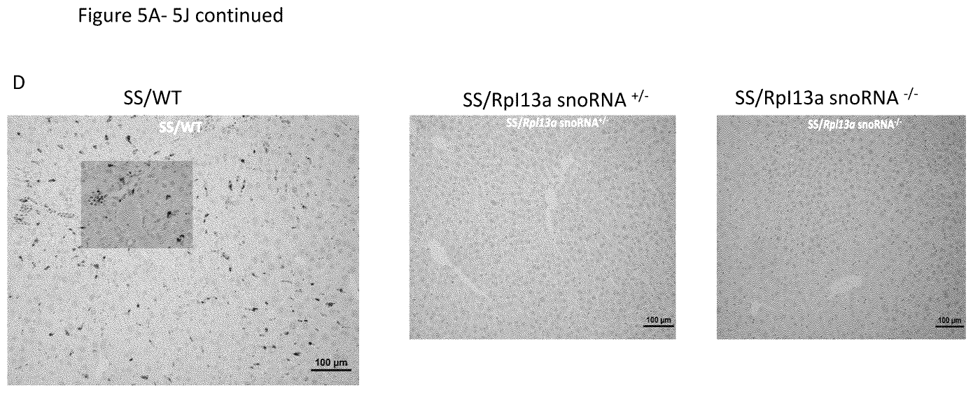

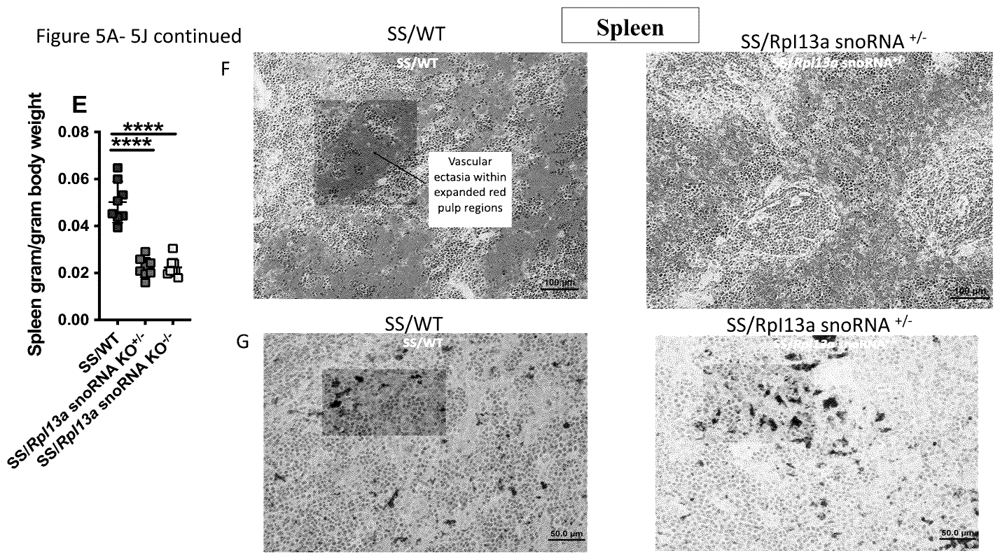

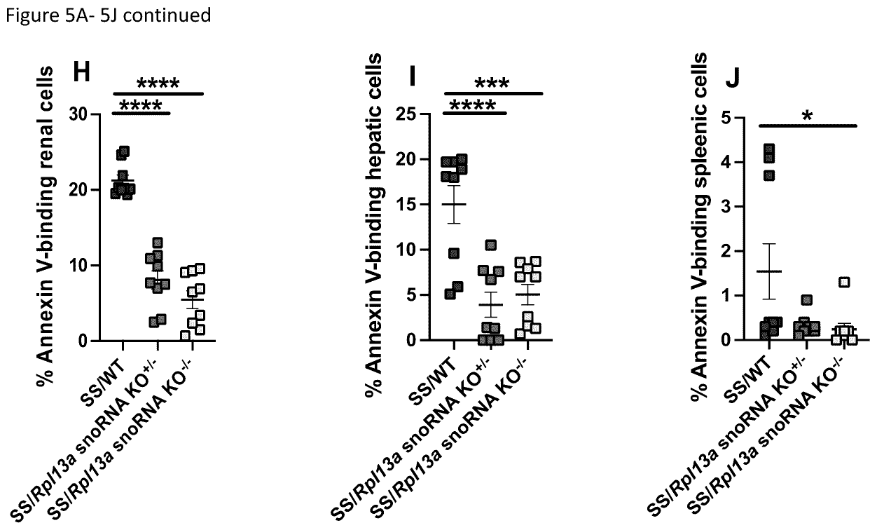

Subsequently, we examined whether RPL13A snoRNAs loss and the associated reductions in ROS can ameliorate kidney, liver, and spleen pathology in our SCD mouse model (n=11/genotype). In the kidney, H&E staining showed prominent glomerulopathy in all SS/WT mice, with glomerular tuft enlargement, hypercellularity, and capillary dilatation with erythrocytic congestion (FIG. 5A). Endothelial cell hypertrophy was noted in blood vessels and capillaries of the cortex and corticomedullary junctional regions. Based on review by an expert pathologist (J. I. E), SS/Rpl13a snoRNA^{+/-} and SS/Rpl13a snoRNA^{-/-} (FIG. 5A) mice had fewer pathologic changes (glomerular, endothelial, and capillary) within the renal cortical parenchyma than SS/WT mice. Prussian blue staining for iron deposition was present in the proximal tubular epithelium of outer cortices in all SS/WT mice, but absent in the snoRNA KO animals (FIG. 5B).

H&E liver sections of all SS/WT mice showed similar numbers and severity of lesions, characterized by large foci of hepatocytic necrosis with circumscribing mononuclear cell inflammation, consistent with infarction, numerous hepatic sinusoids greatly dilated and completely congested with erythrocytes (FIG. 5C). SS/WT mice also showed scattered foci of extramedullary erythropoiesis, and extensive brown pigment consistent with hemosiderin, present in macrophages and Kupffer cells in affected necrotic regions, as well as in lining nearby sinusoids. All SS/Rpl13a snoRNA^{+/-} and SS/Rpl13a snoRNA^{-/-} mice had complete absence of hepatic infarcts (FIG. 5C), though 2 animals in the knockout groups had occasional small foci of mononuclear inflammatory cells or extramedullary hematopoiesis scattered in hepatic tissues. Prussian blue staining confirmed iron deposition in SS/WT livers, as opposed to SS/Rpl13a snoRNA^{+/-} or SS/Rpl13a snoRNA^{-/-} livers (FIG. 5D).

Splenomegaly is a classic finding in SCD. Depleting snoRNAs reduced by half spleen size relative to body weight for SS/Rpl13a snoRNA^{+/-} and SS/Rpl13a snoRNA^{-/-} mice compared to SS/WT mice (n=8 per group; FIG. 5E). H&E staining of spleen sections showed vascular ectasia within expanded red pulp region and marked extramedullary hematopoiesis in all SS/WT mice (n=11; FIG. 5F). Two SS/WT mice had evidence of splenic infarction. SS/Rpl13a snoRNA^{+/-} (n=11; FIG. 5F) and SS/Rpl13a snoRNA^{-/-} (n=11) genotypes were devoid of vascular ectasia, but they had red pulp congestion and extramedullary hematopoiesis. Prussian blue staining revealed iron deposition in all genotypes, but accumulation of hemosiderin in red pulp regions was much more accentuated in the SS/WT group (FIG. 5G).

To provide another quantitative assessment of how the RPL13A snoRNAs contribute to organ pathology in sickle mice, we used Annexin V staining to determine early cellular apoptosis (Annexin V positive; n=9/genotype) by flow cytometry. Although the generation of single-cell suspensions from tissues is itself a significant cellular stress, the use of flow cytometry of one hundred thousand of cells from each organ allows for accurate assessment, revealing the relative propensity of each cellular genotype to undergo apoptosis with high confidence. SS/WT mice showed high levels of Annexin V positive cells in the kidneys (FIG. 5H) and liver (FIG. 5I), with some animals also having increased sensitivity to apoptosis in the spleen (FIG. 5J). Compared to SS/WT mice, SS/Rpl13a snoRNA^{+/-} and SS/Rpl13a snoRNA^{-/-} mice had significantly reduced early apoptosis in the kidney (p<0.0001) and liver (p<0.001), and/or spleen (p<0.05). In aggregate, our data suggest that reducing RPL13A snoRNA expression improves cellular and organ tissue health in SCD.

RPL13A snoRNA Depletion Ameliorates Leukocytosis and Anemia.

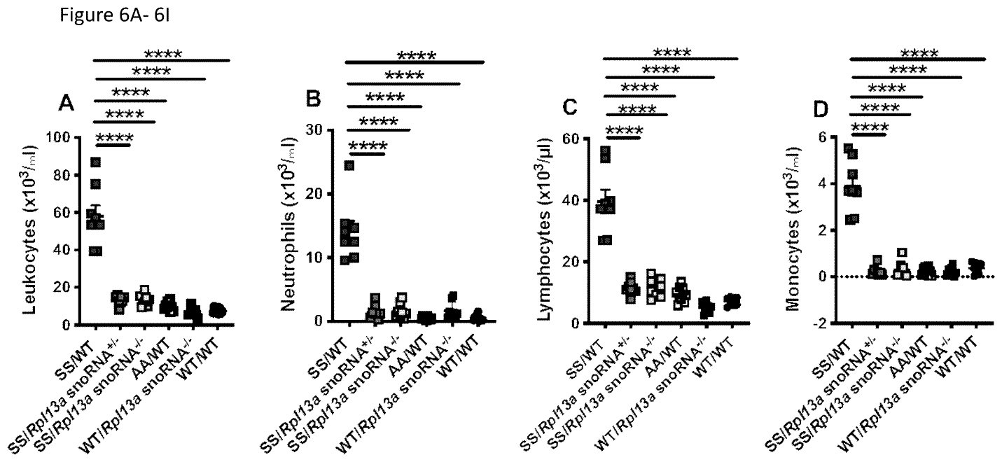

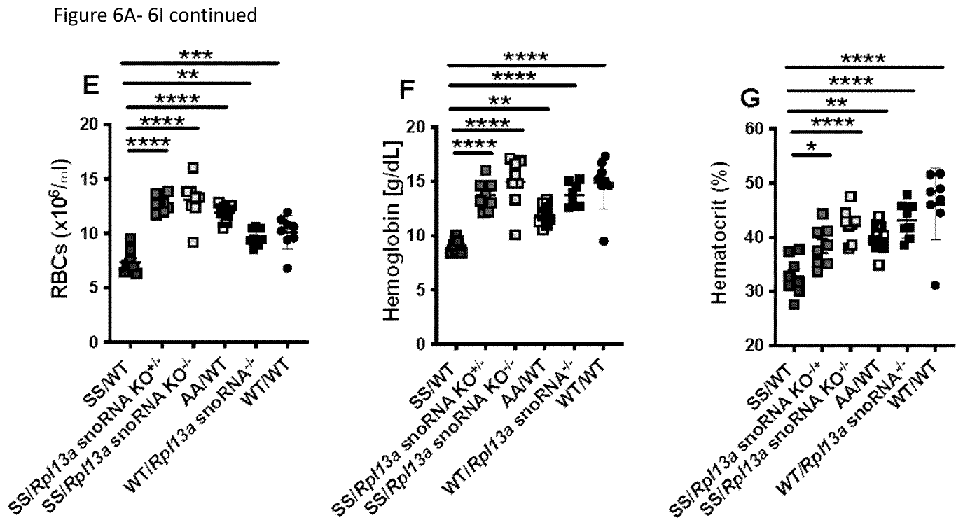

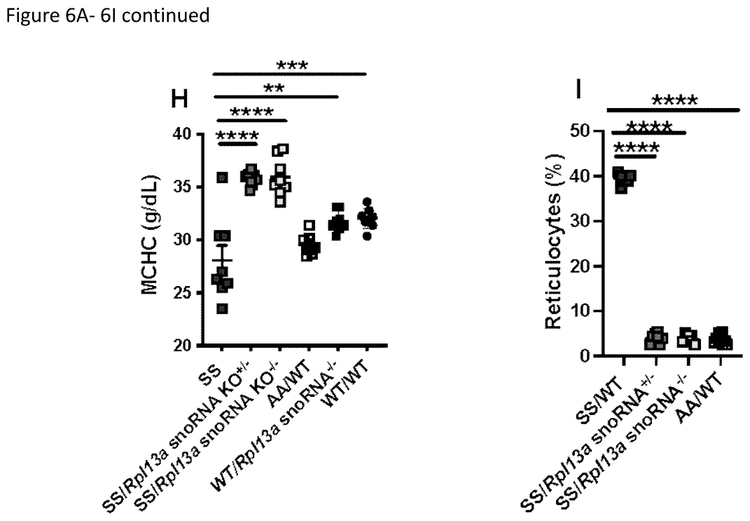

In SCD, leukocytosis is associated with increases in the incidence of pain crisis, acute chest syndrome, stroke, and mortality³⁸, and neutrophil counts in particular, are increased in patients and in sickle mice^{13, 38-40}. We evaluated whether RPL13A snoRNA loss affects peripheral blood cell counts in both the sickle and WT mice. SS/Rpl13a snoRNA^{+/-}, SS/Rpl13a snoRNA^{-/-}, and WT/Rpl13a snoRNA^{-/-} mice showed normalized leukocyte, neutrophil, lymphocyte, and monocyte counts, which were close to or within the ranges observed in the controls AA/WT, and WT/WT mice (p<0.0001; FIG. 6A-D).

We also assessed the effect of RPL13A snoRNA loss on RBC parameters since oxidative stress contributes to the pathophysiology of anemia in SCD. Rpl13a snoRNA loss did not affect RBC parameters in WT mice (FIG. 6E-H). However and in comparison with SS/WT mice (p<0.0001), RBC counts, Hb, and hematocrit rose in SS/Rpl13a snoRNA^{+/-} and SS/Rpl13a snoRNA^{-/-} mice to levels, which were within the ranges detected in AA/WT mice (FIG. 6E-G). MCHC was also significantly higher in SS/Rpl13a snoRNA^{+/-}, SS/Rpl13a snoRNA^{-/-}, and the controls WT/Rpl13a snoRNA^{-/-} and WT/WT mice vs. SS/WT mice (p<0.0001; FIG. 6H). In contrast, reticulocyte counts were significantly lower in SS/Rpl13a snoRNA^{+/-}, SS/Rpl13a snoRNA^{-/-}, and the controls AA/WT mice compared to SS/WT mice (p<0.0001; FIG. 6I). Thus, knocking-out RPL13A snoRNAs reduces leukocytosis and improves anemia, which subsequently may ameliorate inflammation in SCD.

Depletion in RPL13A snoRNAs Reduces Vaso-Occlusion Following an Inflammatory Trigger of Vaso-Occlusion in Sickle Mice.

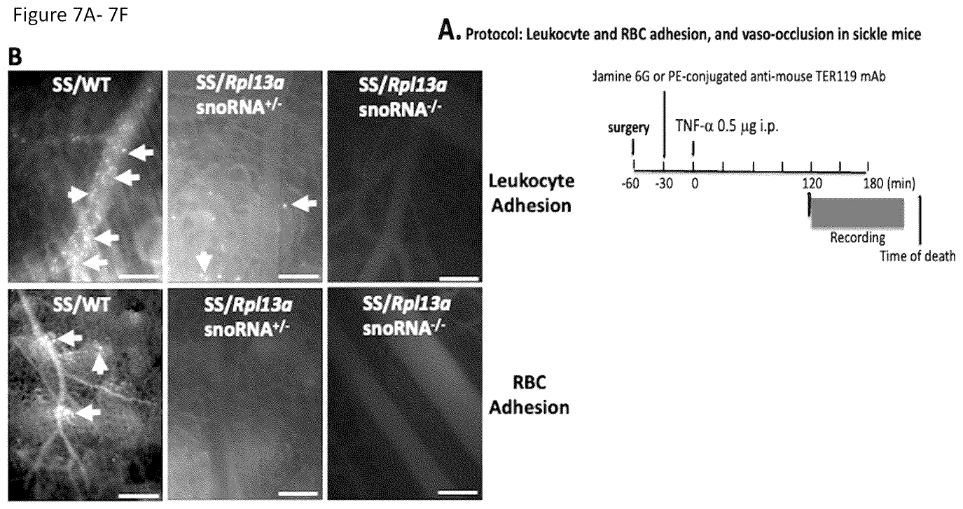

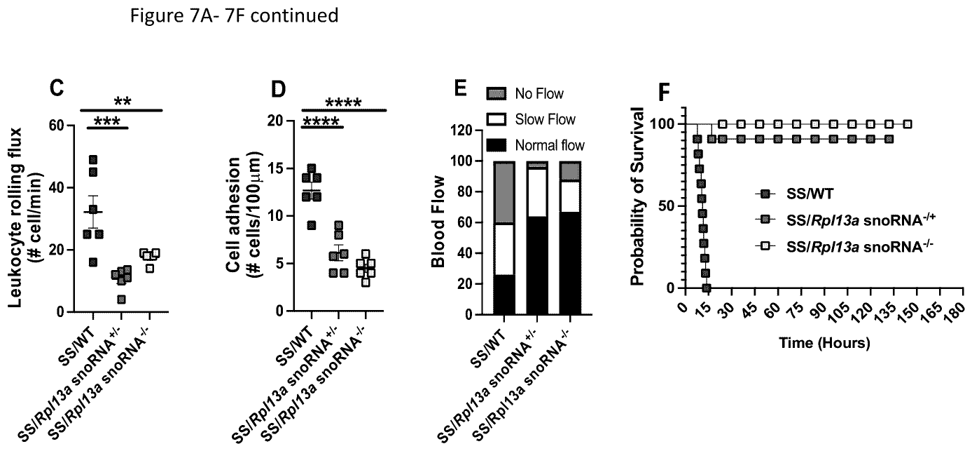

Since RPL13A snoRNA loss in sickle mice reduced both sickle RBC and tissue ROS levels and oxidative stress, thereby improving SCD pathology, we consequently addressed the effect of knocking-out RPL13A snoRNAs in sickle mice on vaso-occlusion triggered by administering the pro-inflammatory cytokine, TNF-α⁴¹. To monitor the circulatory behavior of fluorescence-labeled RBCs and fluorescence-labeled leukocytes in our sickle mouse model, we performed intravital microscopy 120 minutes post-TNF-α, injection (Protocol, FIG. 7A), a time during which leukocytes are recruited ad adhered to the endothelium, sickle RBCs are adherent to adherent leukocytes and endothelium, and a vaso-occlusion-like process is induced. Intravital microscopy revealed high numbers of constantly rolling leukocytes (an average of 45 leukocytes rolling/min) in SS/WT mice, some of which became adherent (FIG. 7B-C). Conversely, the average number of rolling leukocytes declined by 70% (p=0.0027), and 55% (p=0.0259) in SS/Rpl13a snoRNA^{+/-} and SS/Rpl13a snoRNA^{-/-} mice, respectively, compared to the control group (FIG. 7B-C). SS/WT mice also showed avid cell, leukocyte and RBC, adhesion in inflamed vessels, which were sustained overtime (FIG. 7B), leading to intermittent and transient occlusion of multiple venule segments as reflected by blood stasis. Contrariwise, heterozygous, and homozygous RPL13A snoRNA KO in sickle mice blunted adhesion of leukocytes and RBCs, and vaso-occlusion compared to the control group (FIG. 7B). The number of adherent sickle RBCs and leukocytes over 100 μm venular lengths decreased by 47% (p=0.0014), and 64% (p<0.0001) in SS/Rpl13a snoRNA^{+/-} and SS/Rpl13a snoRNA^{-/-} groups, respectively, compared to SS/WT mice (FIG. 7D). Inhibition of leukocyte rolling and cell adhesion in SS/Rpl13a snoRNA^{+/-} and SS/Rpl13a snoRNA^{-/-} mice led to preservation of blood flow in 64% and 67% of the total vessels recorded, respectively, as opposed to only 26% of the venules with normal blood flow in the control group (FIG. 7E).

TNF-α-induced vaso-occlusion in sickle mice leads to death within several hours. To assess the effect of RPL13A snoRNA KO in sickle mice on mortality under crisis condition, sickle mice were monitored for more than 1 week after the TNF-α challenge. Kaplan-Meier survival curves showed 90.91% and 100% survival of SS/Rpl13a snoRNA^{+/-} and SS/Rpl13a snoRNA^{-/-} mice, respectively, under TNF-α-induced crisis condition compared with the controls (FIG. 7F; p=0.00001, log-rank test). The median survival in the control group was 11.08 hours after TNF-α injection, suggesting that occlusion of some vessel segments caused by inflammation was likely persistent in the control group leading to animal death. Together, these results strongly argue that heterozygous RPL13A snoRNA KO prevents an inflammatory trigger from initiating and/or exacerbating vaso-occlusion in SCD mice, by at least negatively regulating sickle RBC and activated leukocyte adhesion.

### Discussion

Our in vivo data reveal that depleting RPL13A snoRNAs in a mouse model of SCD alleviates organ damage, and above all ameliorates anemia and prevents an inflammatory insult from precipitating vaso-occlusion and shortening survival rate.

Consistent with a primary role for cytoplasmic RPL13A snoRNAs, high RPL13A snoRNA expression in human SS RBCs was associated with both excessive ROS production and low peroxidase activity. Knocking-out these snoRNAs in sickle mice lowered murine sickle RBC ROS production, implicating the RPL13A snoRNAs as mediators of RBC ROS generation^{11, 42}. Our previous studies have evidenced that ROS production in human SS RBCs is dependent on Nox activation, and that Nox-dependent ROS generation creates a positive feedback loop with GRK2, and ERK1/2¹⁸. GRK2 and ERK1/2 were more abundant in SS than in AA RBCs^{18, 35}, along with Nox1 expression, while the activity of peroxidases, enzymes neutralizing H₂O₂, is diminished in these sickle cells compared to AA RBCs. Sickle RBC RPL13A snoRNAs were found to regulate ROS production by controlling the abundance of potential mRNA targets, NOX1, NOX4, GRK2, ERK1 and ERK2, Nox2 protein levels, and ERK1/2 and peroxidase activities. Depleting sickle RBC RPL13A snoRNAs in sickle mice down-regulated NOX1, GRK2, ERK1 and ERK2 mRNA and their respective protein expression levels, Nox2 protein expression along with ROS levels. In contrast, Nox4 expression along with its detected product H₂O₂ and the peroxidase activity all increased in murine sickle cells because of RPL13A snoRNA knockout to levels comparable to those detected in RBCs in AA/WT mice. Down-regulation in RBC Nox1 and Nox2 expression seems to account for reduced cytotoxic ROS in particular, whereas up-regulation in RBC peroxidase activity can lower the steady-state pool of both ROS and H₂O₂ in SS/WT and AA/WT mice.

The fact that the RPL13A snoRNAs are present in sickle erythroid cells to regulate NOX1, NOX4, GRK2, ERK1 and ERK2 transcript levels and/or protein levels, confirms once more that these snoRNAs function in roles beyond the processing and modification of ribosomal RNAs. Genetic studies have demonstrated that the RPL13A snoRNAs have a dynamic presence in the cytoplasm, where elevated cytosolic levels of these ncRNAs correspond to elevated ROS levels and oxidative stress⁴². RPL13A snoRNAs were suggested to function though non-canonical mode of actions, involving trafficking to the cytoplasm⁴². Localization of RPL13A snoRNAs in the enucleated sickle cells further suggests that these ncRNAs may play non-canonical role targeting enzymes of oxidative stress. RPL13A snoRNAs may shuttle between the nucleus and cytoplasm⁴³, and they may have a role outside the nuclear structure to regulate the mRNA or protein levels, and thereby activity. Accumulation of snoRNAs in the cytoplasm^{42, 44} is consistent with regulation of snoRNA function by changes in localization and thus proximity to its target, such as RBC NOX2 mRNA in our case. While the mechanisms by which these ncRNAs regulate the different enzyme/kinase expression in sickle RBCs are currently under investigation, possible mechanisms include the snoRNAs interacting with the protein machinery to regulate directly or indirectly the abundance and/or translation efficiency of the above described oxidative stress-related mRNAs⁴⁵⁻⁴⁷ likely during erythroid differentiation. Also, we cannot exclude that diminished ROS levels in RPL13A snoRNA depleted sickle RBCs may contribute to the downregulation of GRK2 and ERK1/2 expression levels/activity.

Clinically, we have extended our findings and determined the effect of depleting RPL13A snoRNAs on leukocytosis, anemia, vaso-occlusion, and mortality. Oxidative stress in sickle RBCs is increasingly accepted as both a driving force of hemolysis⁴⁸ and a trigger of vaso-occlusion^{22, 37, 49} and leukocytosis is associated with increased incidences of pain crisis and mortality³⁸. Reduction in RPL13A snoRNA expression in sickle mice ameliorated anemia, and mitigated not only leukocytosis, but also adhesion of sickle RBCs and leukocytes, and vaso-occlusion initiated by an inflammatory insult, and preserved normal blood flow. These positive clinical outcomes were due to the marked decrease in snoRNAs-regulated ROS production and oxidative stress in sickle RBCs and possibly in the endothelium and leukocytes. Leukocytes, neutrophils in particular, have an important role in vaso-occlusion by interacting with the endothelium and sickle RBCs. Cycles of vaso-occlusion can trigger inflammation⁴⁹ and redox instability that result in a constant state of disruption to the vascular system. This leads to recurrent episodes of ischemia-reperfusion injury to multiple organs and infarction, thus generating more ROS, which exacerbates the clinical manifestations in SCD patients^{50, 51}. We also suggest that improved anemia, by reducing RBC ROS levels, and thus hemolysis, contributes to alleviation of endothelial dysfunction and oxidative organ damage in sickle mice. By collectively reducing RBC oxidative stress-induced vaso-occlusion, and improving vascular dysfunction, animal survival rate under TNFα-induced crisis condition was ameliorated. These data together support the notion that deleting RPL13A snoRNAs could have beneficial outcomes, ameliorating detrimental downstream mechanisms and improving key pathological markers causing the deleterious SCD complications. It has been known that SCD patients suffer from severe anemia, pain crisis, and chronic inflammatory state, in which multiple inflammatory mediators and acute inflammatory cells are elevated⁵²⁻⁵⁵. Lung inflammation and vaso-occlusion cause acute chest syndrome, which is one of the main leading causes of death in SCD patients⁵⁵⁻⁵⁷. Pain crises also contribute to acute kidney injury, and SCD patients who develop nephropathy are at high risk for mortality⁵⁸. Thus, ameliorating anemia, and preventing inflammatory insults-induced pain crises by genetically targeting RPL13A snoRNAs may represent a powerful therapeutic approach for treating SCD pathology.

### REFERENCES

1. Holley C L and Topkara V K. An introduction to small non-coding RNAs: miRNA and snoRNA. Cardiovascular drugs and therapy sponsored by the International Society of Cardiovascular Pharmacotherapy. 2011; 25:151-9.
2. Tollervey D and Kiss T. Function and synthesis of small nucleolar RNAs. Curr Opin Cell Biol. 1997; 9:337-42.
3. Clouet d'Orval B, Bortolin M L, Gaspin C and Bachellerie J P. Box C/D RNA guides for the ribose methylation of archaeal tRNAs. The tRNATrp intron guides the formation of two ribose-methylated nucleosides in the mature tRNATrp. Nucleic Acids Res. 2001; 29:4518-29.
4. Kiss T, Fayet-Lebaron E and Jady B E. Box H/ACA small ribonucleoproteins. Molecular cell. 2010; 37:597-606.
5. McKeegan K S, Debieux C M, Boulon S, Bertrand E and Watkins N J. A dynamic scaffold of pre-snoRNP factors facilitates human box C/D snoRNP assembly. Molecular and cellular biology. 2007; 27:6782-93.
6. Decatur W A and Fournier M J. rRNA modifications and ribosome function. Trends Biochem Sci. 2002; 27:344-51.
7. Kiss-Laszlo Z, Henry Y, Bachellerie J P, Caizergues-Ferrer M and Kiss T. Site-specific ribose methylation of preribosomal RNA: a novel function for small nucleolar RNAs. Cell. 1996; 85:1077-88.
8. Kiss A M, Jady B E, Darzacq X, Verheggen C, Bertrand E and Kiss T. A Cajal body-specific pseudouridylation guide RNA is composed of two box H/ACA snoRNA-like domains. Nucleic Acids Res. 2002; 30:4643-9.
9. Wang C and Meier U T. Architecture and assembly of mammalian H/ACA small nucleolar and telomerase ribonucleoproteins. EMBO J. 2004; 23:1857-67.
10. Reichow S L, Hamma T, Ferre-D'Amare A R and Varani G. The structure and function of small nucleolar ribonucleoproteins. Nucleic Acids Res. 2007; 35:1452-64.
11. Michel C I, Holley C L, Scruggs B S, Sidhu R, Brookheart R T, Listenberger L L, Behlke M A, Ory D S and Schaffer J E. Small nucleolar RNAs U32a, U33, and U35a are critical mediators of metabolic stress. Cell Metab. 2011; 14:33-44.
12. Lee J, Harris A N, Holley C L, Mahadevan J, Pyles K D, Lavagnino Z, Scherrer D E, Fujiwara H, Sidhu R, Zhang J, Huang S C, Piston D W, Remedi M S, Urano F, Ory D S and Schaffer J E. Rpl13a small nucleolar RNAs regulate systemic glucose metabolism. J Clin Invest. 2016; 126:4616-4625.
13. Emokpae M A, Uadia P O and Gadzama A A. Correlation of oxidative stress and inflammatory markers with the severity of sickle cell nephropathy. Annals of African medicine. 2010; 9:141-6.
14. Nur E, Biemond B J, Otten H M, Brandjes D P, Schnog J J and Group C S. Oxidative stress in sickle cell disease; pathophysiology and potential implications for disease management. American journal of hematology. 2011; 86:484-9.
15. Rusanova I, Escames G, Cossio G, de Borace R G, Moreno B, Chahboune M, Lopez L C, Diez T and Acuna-Castroviejo D. Oxidative stress status, clinical outcome, and beta-globin gene cluster haplotypes in pediatric patients with sickle cell disease. European journal of haematology. 2010; 85:529-37.
16. Hebbel R P, Eaton J W, Balasingam M and Steinberg M H. Spontaneous oxygen radical generation by sickle erythrocytes. J Clin Invest. 1982; 70:1253-9.
17. George A, Pushkaran S, Konstantinidis D G, Koochaki S, Malik P, Mohandas N, Zheng Y, Joiner C H and Kalfa T A. Erythrocyte NADPH oxidase activity modulated by Rac G TPases, PKC, and plasma cytokines contributes to oxidative stress in sickle cell disease. Blood. 2013; 121:2099-107.
18. MacKinney A, Woska E, Spasojevic I, Batinic-Haberle I and Zennadi R. Disrupting the vicious cycle created by NOX activation in sickle erythrocytes exposed to hypoxia/reoxygenation prevents adhesion and vasoocclusion. Redox biology. 2019:101097.
19. Mohandas N and Evans E. Sickle erythrocyte adherence to vascular endothelium. Morphologic correlates and the requirement for divalent cations and collagen-binding plasma proteins. J Clin Invest. 1985; 76:1605-12.
20. Hofstra T C, Kalra V K, Meiselman H J and Coates T D. Sickle erythrocytes adhere to polymorphonuclear neutrophils and activate the neutrophil respiratory burst. Blood. 1996; 87:4440-7.
21. Zennadi R, Chien A, Xu K, Batchvarova M and Telen M J. Sickle red cells induce adhesion of lymphocytes and monocytes to endothelium. Blood. 2008; 112:3474-83.
22. Kaul D K, Liu X D, Zhang X, Ma L, Hsia C J and Nagel R L. Inhibition of sickle red cell adhesion and vasoocclusion in the microcirculation by antioxidants. American journal of physiology Heart and circulatory physiology. 2006; 291:H167-75.
23. Bernaudin F, Verlhac S, Chevret S, Torres M, Coic L, Arnaud C, Kamdem A, Hau I, Grazia Neonato M and Delacourt C. G6PD deficiency, absence of alpha-thalassemia, and hemolytic rate at baseline are significant independent risk factors for abnormally high cerebral velocities in patients with sickle cell anemia. Blood. 2008; 112:4314-7.
24. Sachdev V, Kato G J, Gibbs J S, Barst R J, Machado R F, Nouraie M, Hassell K L, Little J A, Schraufnagel D E, Krishnamurti L, Novelli E M, Girgis R E, Morris C R, Rosenzweig E B, Badesch D B, Lanzkron S, Castro O L, Taylor J Gt, Hannoush H, Goldsmith J C, Gladwin M T and Gordeuk V R. Echocardiographic markers of elevated pulmonary pressure and left ventricular diastolic dysfunction are associated with exercise intolerance in adults and adolescents with homozygous sickle cell anemia in the United States and United Kingdom. Circulation. 2011; 124:1452-60.
25. Balkaran B, Char G, Morris J S, Thomas P W, Serjeant B E and Serjeant G R. Stroke in a cohort of patients with homozygous sickle cell disease. The Journal of pediatrics. 1992; 120:360-6.
26. Hebbel R P. Beyond hemoglobin polymerization: the red blood cell membrane and sickle disease pathophysiology. Blood. 1991; 77:214-37.
27. Hebbel R P, Boogaerts M A, Eaton J W and Steinberg M H. Erythrocyte adherence to endothelium in sickle-cell anemia. A possible determinant of disease severity. N Engl J Med. 1980; 302:992-5.
28. Powars D R, Elliott-Mills D D, Chan L, Niland J, Hiti A L, Opas L M and Johnson C. Chronic renal failure in sickle cell disease: risk factors, clinical course, and mortality. Annals of internal medicine. 1991; 115:614-20.
29. Levasseur D N, Ryan T M, Pawlik K M and Townes T M. Correction of a mouse model of sickle cell disease: lentiviral/antisickling beta-globin gene transduction of unmobilized, purified hematopoietic stem cells. Blood. 2003; 102:4312-9.
30. Townes T M, Ryan T M, Behringer R R, Palmiter R D and Brinster R L. DNase I super-hypersensitive sites direct high level erythroid expression of human alpha-, beta- and beta s-globin genes in transgenic mice. Progress in clinical and biological research. 1989; 316A:47-61.
31. Ryan T M, Ciavatta D J and Townes T M. Knockout-transgenic mouse model of sickle cell disease. Science. 1997; 278:873-6.
32. Ryan T M, Townes T M, Reilly M P, Asakura T, Palmiter R D, Brinster R L and Behringer R R. Human sickle hemoglobin in transgenic mice. Science. 1990; 247:566-8.
33. Thamilarasan M, Estupinan R, Batinic-Haberle I and Zennadi R. Mn porphyrins as a novel treatment targeting sickle cell NOXs to reverse and prevent acute vaso-occlusion in vivo. Blood Adv. 2020; 4:2372-2386.
34. Zennadi R, Moeller B J, Whalen E J, Batchvarova M, Xu K, Shan S, Delahunty M, Dewhirst M W and Telen M J. Epinephrine-induced activation of LW-mediated sickle cell adhesion and vaso-occlusion in vivo. Blood. 2007; 110:2708-17.
35. Zennadi R, Whalen E J, Soderblom E J, Alexander S C, Thompson J W, Dubois L G, Moseley M A and Telen M J. Erythrocyte plasma membrane-bound ERK1/2 activation promotes ICAM-4-mediated sickle red cell adhesion to endothelium. Blood. 2012; 119:1217-27.
36. Nath K A, Grande J P, Haggard J J, Croatt A J, Katusic Z S, Solovey A and Hebbel R P. Oxidative stress and induction of heme oxygenase-1 in the kidney in sickle cell disease. The American journal of pathology. 2001; 158:893-903.
37. Mahaseth H, Vercellotti G M, Welch T E, Bowlin P R, Sonbol K M, Hsia C J, Li M, Bischof J C, Hebbel R P and Belcher J D. Polynitroxyl albumin inhibits inflammation and vasoocclusion in transgenic sickle mice. The Journal of laboratory and clinical medicine. 2005; 145:204-11.
38. Wun T. The Role of Inflammation and Leukocytes in the Pathogenesis of Sickle Cell Disease; Haemoglobinopathy. Hematology. 2001; 5:403-412.
39. Belcher J D, Mahaseth H, Welch T E, Otterbein L E, Hebbel R P and Vercellotti G M. Heme oxygenase-1 is a modulator of inflammation and vaso-occlusion in transgenic sickle mice. J Clin Invest. 2006; 116:808-16.
40. Beckman J D, Belcher J D, Vineyard J V, Chen C, Nguyen J, Nwaneri M O, O'Sullivan M G, Gulbahce E, Hebbel R P and Vercellotti G M. Inhaled carbon monoxide reduces leukocytosis in a murine model of sickle cell disease. American journal of physiology Heart and circulatory physiology. 2009; 297:H1243-53.
41. Hebbel R P, Osarogiagbon R and Kaul D. The endothelial biology of sickle cell disease: inflammation and a chronic vasculopathy. Microcirculation. 2004; 11:129-51.
42. Holley C L, Li M W, Scruggs B S, Matkovich S J, Ory D S and Schaffer J E. Cytosolic accumulation of small nucleolar RNAs (snoRNAs) is dynamically regulated by NADPH oxidase. J Biol Chem. 2015; 290:11741-8.
43. Baserga S J, Gilmore-Hebert M and Yang X W. Distinct molecular signals for nuclear import of the nucleolar snRNA, U3. Genes Dev. 1992; 6:1120-30.
44. Sienna N, Larson D E and Sells B H. Altered subcellular distribution of U3 snRNA in response to serum in mouse fibroblasts. Experimental cell research. 1996; 227:98-105.
45. Falaleeva M, Pages A, Matuszek Z, Hidmi S, Agranat-Tamir L, Korotkov K, Nevo Y, Eyras E, Sperling R and Stamm S. Dual function of C/D box small nucleolar RNAs in rRNA modification and alternative pre-mRNA splicing. Proc Natl Acad Sci USA. 2016; 113:E1625-34.
46. Schwartz S, Bernstein D A, Mumbach M R, Jovanovic M, Herbst R H, Leon-Ricardo B X, Engreitz J M, Guttman M, Satija R, Lander E S, Fink G and Regev A. Transcriptome-wide mapping reveals widespread dynamic-regulated pseudouridylation of ncRNA and mRNA. Cell. 2014; 159:148-162.
47. Elliott B A, Ho H T, Ranganathan S V, Vangaveti S, Ilkayeva O, Abou Assi H, Choi A K, Agris P F and Holley C L. Modification of messenger RNA by 2′-O-methylation regulates gene expression in vivo. Nat Commun. 2019; 10:3401.
48. Hebbel R P, Morgan W T, Eaton J W and Hedlund B E. Accelerated autoxidation and heme loss due to instability of sickle hemoglobin. Proc Natl Acad Sci USA. 1988; 85:237-41.
49. Belcher J D, Chen C, Nguyen J, Zhang P, Abdulla F, Nguyen P, Killeen T, Xu P, O'Sullivan G, Nath K A and Vercellotti G M. Control of Oxidative Stress and Inflammation in Sickle Cell Disease with the Nrf2 Activator Dimethyl Fumarate. Antioxidants & redox signaling. 2017; 26:748-762.
50. Li C, Wright M M and Jackson R M. Reactive species mediated injury of human lung epithelial cells after hypoxia-reoxygenation. Exp Lung Res. 2002; 28:373-89.
51. Schaer D J, Buehler P W, Alayash A I, Belcher J D and Vercellotti G M. Hemolysis and free hemoglobin revisited: exploring hemoglobin and hemin scavengers as a novel class of therapeutic proteins. Blood. 2013; 121:1276-84.
52. Belcher J D, Marker P H, Weber J P, Hebbel R P and Vercellotti G M. Activated monocytes in sickle cell disease: potential role in the activation of vascular endothelium and vaso-occlusion. Blood. 2000; 96:2451-9.
53. Makis A C, Hatzimichael E C, Mavridis A and Bourantas K L. Alpha-2-macroglobulin and interleukin-6 levels in steady-state sickle cell disease patients. Acta haematologica. 2000; 104:164-8.
54. Baum K F, Dunn D T, Maude G H and Serjeant G R. The painful crisis of homozygous sickle cell disease. A study of the risk factors. Archives of internal medicine. 1987; 147:1231-4.
55. Platt O S, Brambilla D J, Rosse W F, Milner P F, Castro O, Steinberg M H and Klug P P. Mortality in sickle cell disease. Life expectancy and risk factors for early death. N Engl J Med. 1994; 330:1639-44.
56. Gray A, Anionwu E N, Davies S C and Brozovic M. Patterns of mortality in sickle cell disease in the United Kingdom. Journal of clinical pathology. 1991; 44:459-63.
57. Thomas A N, Pattison C and Serjeant G R. Causes of death in sickle-cell disease in Jamaica. Br Med J (Clin Res Ed). 1982; 285:633-5.
58. Baddam S, Aban I, Hilliard L, Howard T, Askenazi D and Lebensburger J D. Acute kidney injury during a pediatric sickle cell vaso-occlusive pain crisis. Pediatr Nephrol. 2017; 32:1451-1456.

### Example II—Post-Transcription Regulation by Rpl13a snoRNAs in Sickle Cells Induces Oxidative Stress-Mediated Vaso-Occlusion In Vivo

### Introduction

Oxidative stress caused by increased reactive oxygen species (ROS) production, is an important feature of sickle cell disease (SCD)¹, and plays a significant role in the pathophysiology of vaso-occlusion²⁻⁴. Sickle RBCs are the primary source of ROS in SCD patients⁵⁻⁷. ROS generated by sickle RBCs trigger RBC interactions with the vascular endothelium and other blood cells to obstruct blood vessels^{3, 8-11}. These RBC-cell adhesive interactions cause severe health complications including recurrent pain crises¹²⁻¹⁷. SCD phenotypic severity could therefore be alleviated by reducing oxidative stress and thereby reducing RBC-cell interactions promoting vaso-occlusion¹⁸.

Elevated ROS levels have also been associated with elevated levels of small non-coding nucleolar RNAs (snoRNAs) encoded by the Rpl13a locus¹⁹. This locus codes for the ribosomal protein L13a, which is a component of the large ribosomal subunit, as well as four intronic box C/D snoRNAs: U32a, U33, U34, and U35a. As their name implies, snoRNAs are primarily found in the nucleolus, where ribosomal RNA is synthesized. The Box C/D snoRNAs form sno-ribonucleo-proteins (snoRNPs) via transient interactions with four core proteins: fibrillarin, Nop56, Nop58, and 15.5K²⁰⁻²³. The snoRNA then guides the snoRNP machinery to a target RNA and interact with the target through antisense complementarity sequence. The box C/D snoRNAs are primarily known to guide post-transcriptional modifications, especially 2′-O-methylation, of ribosomal RNA (rRNA) and small nuclear RNA (snRNA)^{24, 25}.

ROS accumulation in sickle RBCs mediating adhesion and vaso-occlusion^{18, 26, 27}, involves partially activation of NADPH oxidases (Nox), enzymes producing superoxide and hydrogen peroxide (H₂O₂)^{2, 3}, the major detected product of the constitutively active Nox4²⁸. Rpl13a snoRNAs have also been shown to accumulate in the cytosol, suggesting that they can function non-canonically¹⁹ by targeting mRNAs outside the nuclear structure. Although the exact mechanisms linking Rpl13a snoRNAs to ROS regulation remain somewhat unclear, recent studies have shown that these snoRNAs can guide 2′-O-methylation on the peroxidasin mRNA²⁹. Therefore, although the snoRNAs are known to guide 2′-O-methylation on rRNA, they may be regulating ROS production though alternative modification sites on mRNA.

Given the pathologic role of ROS in SCD, and the fundamental regulation of ROS by Rpl13a snoRNAs, we hypothesized that sickle RBCs retain cytosolic Rpl13a snoRNAs that contribute to high ROS levels, and therefore mediate RBC adhesion and vaso-occlusion, by regulating cytosolic mRNAs associated with oxidative stress. In this study, we identified the mechanisms by which Rpl13a snoRNAs regulate ROS accumulation in sickle RBCs to promote oxidative stress-mediated vaso-occlusion, and we suggest that therapeutic targeting combinations of these non-coding (nc)RNAs with antisense oligonucleotides is effective in a mouse model of SCD.

### Materials and Methods

Cell culture. K562 (ATCC CCL-243), and K562 CRISPR knockout (KO) clones were maintained in RPMI 1640 (ATCC) supplemented with 10% FBS (Corning) and 2 mM glutamine.

Mice. The Institutional Animal Care and Use Committee and the Committee on the Ethics of Animal Experiments at Duke University approved this animal work. Sickle mice, the Townes mice expressing exclusively human α- and sickle β-globin, and a well-accepted model of human SCD^{30, 31}, were obtained from colonies established by Dr. T. Townes at the University of Alabama^{32, 33} The Rpl3a snoRNA snoRNA-null mice were obtained from colonies established by Dr. J. Schaffer at Washington University in St. Louis³⁴. Sickle mice expressing wild-type (WT) Rpl13a snoRNAs, and sickle mice with a global Rpl13a snoRNA KO referred here as SS/WT, and SS/Rpl13a snoRNA-null mice, respectively, were generated by Dr. Zennadi at Duke University by coupling Townes mouse with Rpl13a snoRNA snoRNA-null mouse. Genotyping was confirmed by PCR. The sickle mouse mutants, 50% female, were used at 10-14 weeks of age. To in vivo knockdown (KD) Rpl13a snoRNAs U34 and U35a, or U32a, U34 and U35a in sickle mice, antisense oligonucleotides (ASOs) U32a-ASO (SEQ ID NO: 44), U34-ASO (SEQ ID NO: 47), and U35a-ASO (SEQ ID NO: 46) were designed to specifically target murine U32a, U34, and U35a, respectively. An ASO targeting sequence from GFP (SEQ ID NO: 48) was used as a control. Sickle mice were then subjected every other day to double-KD of U34 and U35a with three doses at one dose/day of U34-ASO and U35a-ASO, triple-KD of U32a, U34 and U35a with three doses at one dose/day of U32a-ASO, U34-ASO and U35a-ASO, or three doses at one dose/day of GFP-ASO prior to in vivo assays.

Blood collection, and preparation. Blood samples collected from human participants have been approved by Duke University's Institutional Review Board and written informed consent has been obtained from the participants. Blood samples were collected from adult SCD patients homozygous for hemoglobin S, and healthy donors. All SCD patients had not been transfused for at least three months and had not experienced acute vaso-occlusive crises for three weeks. Human and murine Blood samples were collected into citrate tubes, and were used immediately. RBCs were separated centrifugally and washed extensively using phosphate-buffered saline (PBS). Packed RBCs were analyzed for leukocyte and platelet contaminations using an Automated Hematology Analyzer K-1000 (Sysmex Corporation, Kobe, Japan).

Antibodies. Monoclonal and polyclonal antibodies used were purified immunoglobulin [Ig], and they were against the following human or mouse proteins: HuR/ELAV1 (IgG1) (Santa Cruz biotechnology, Dallas, TX), peroxiredoxin 2 (Prdx2) (IgG1) (Proteintech, Rosemont, L); glyceraldehyde 3-phosphate dehydrogenase (GAPDH) (Santa Cruz biotechnology); the control IgG1 (Invitrogen, Carlsbad, CA), and Nox4 (Abcam, Cambridge, MA). Antibodies were used at saturating dilutions.

RT-qPCR. RBC, K562, or organ RNA was isolated using TRIzol reagent (Ambion) according to the manufacturer. cDNAs were prepared using SuperScript® III First-Strand Synthesis kit (Invitrogen). Relative mRNA expression was measured using RT-qPCR and SyberGreen on the StepOnePlus platform and normalized to an endogenous control 36B4 (Rplp0) gene.

ROS and peroxidase activity detection. RBC ROS and H₂O₂ production, and ROS production in K562 cell lines or organs were measured using CM-H2-DCFDA (DCF; Invitrogen, Carlsbad, CA) for ROS detection, and Amplex Red H₂O₂/Peroxidase Assay Kit (Molecular Probes, Grand Island, NY) for H₂O₂ detection as described previously in detail3. One hundred thousand events per sample were acquired and tested by flow cytometric analysis. Peroxidase activity was detected using Amplex Red H₂O₂/Peroxidase Assay Kit following the manufacturer's instructions.

Western blot. RBC and K562 cell line lysates were prepared in RIPA buffer (Sigma R0278) with Complete™ Protease Inhibitor Cocktail (Roche). Protein concentrations were determined using a BCA assay (ThermoFisher). Proteins were separated on 10% SDS-PAGE gels and transferred to nitrocellulose membranes. Immunoblotting was performed with the following antibodies: anti-Nox4 (1:5000), anti-HuR, and anti-Prdx2 (1:500). Images were captured using a ChemDoc XRS+system (Bio-Rad). Protein expression levels were normalized to total to GAPDH expression and analyzed using ImageLab software (Bio-Rad).

Pulldown assays. Cell lysates were incubated with 1-2 μg anti-HuR antibody for 2 hours, then incubated with protein A agarose for 45 minutes, at 4° C. with rotating. Beads were washed with lysis buffer then with cold PBS. RNA was collected from the beads with TRIzol, and Nox4 mRNA and snoRNA expression was determined by RT-qPCR. Protein was collected with Laemmli sample buffer and subjected to Western blots using anti-HuR antibody (1:500).

Reverse transcription at low dNTP followed by PCR (RTL-P). This procedure was adapted from the previously described RTL-P method. cDNA was generated from 500 ng of total RNA, with oligo-dT RT priming, under two conditions: (1) standard SuperScript III (ThermoFisher) reverse transcriptase reaction (1 mM dNTP mix), and (2) under the same conditions except low dNTP concentration (0.1 mM dNTP mix). qPCR was then performed in 20 μL reactions with Power SYBR, 0.25 μM forward and reverse primers, and 2 μL of each cDNA with a hot start (95 C; 10′) and 40 cycles of 95 C (15 s) and 60 C (1′). Relative quantification of Prdx2 from each cDNA reaction (both high and low dNTP) was first normalized to an internal reference gene (Rplp0) to correct for any differences in total RNA input (standard ddCT method). The ratio of low/high dNTP product was then calculated for each condition, to normalize the low dNTP result to the amount of Prdx2 mRNA in each sample. This value could be compared between experimental (KO) conditions as a relative quantity (RQ). This method accounts for changes in transcript methylation relative to transcript abundance in normal conditions.

Window chamber surgery and intravital microscopy. Following anesthesia, all dorsal skin-fold window chamber surgery was carried out on anesthetized mice under sterile conditions with aseptic technique using a laminar flow hood. Animals were placed on a heated pad at 37° C. General anesthesia was achieved with isoflurane (Butler Animal Health Supply, Dublin, OH), by adjusting the oxygen flowmeter to approximately 0.5-1.0 L/min, and isoflurane vaporizer to 3% for induction and 1% for maintenance. The back of anesthetized mouse was alternatively wiped with surgical sponges soaked in Hibiclens and then alcohol. Hair on the clean area was shaved, and the rest of hair was removed using hair removal cream. The area was cleaned again with Hibiclens and then alcohol. A window chamber consisting of a double-sided titanium frame was surgically implanted into the dorsal skin fold. Surgery involved careful removal of the epidermal and dermal layers of one side of a dorsal skin flap, exposing blood vessels of the subcutaneous tissue adjacent to the striated muscles of the opposing skin fold, and then securing the two sides of the chamber to the skin using stainless steel screws and sutures. Sutures were used to secure the window chamber, which will be retained for the duration of the experiment to maintain the window chambers. A glass window was placed in the chamber to cover the exposed tissue and secured with a snap ring.

Intravital microscopy. Anesthetized sickle mice treated with ASOs were injected through a tail vein with 100 μL (0.02% in sterile saline) rhodamine 6G (Sigma-Aldrich, St. Louis, MO) and 0.25 μg per g body weight PE-conjugated anti-mouse TER119 (Ly-76) monoclonal antibody (mAb; San Diego, CA) for in vivo labeling and monitoring of leukocytes and RBCs, respectively. After 30 minutes, mice were injected intra-peritoneal (IP) with 500 ng murine recombinant tumor necrosis alpha (TNF-α) to precipitate vaso-occlusion. Hundred and thirty minutes later, intravital microscopy observation was performed for at least 2 hours. A minimum of 20 venules of each mouse were recorded. Adhesion of RBCs, and leukocytes was quantified by counting the number of adherent cells (stationary cells for longer than 30 seconds) and RBCs (RBC arrest for >0.07 s) along the length of a given venule, and expressed as average number of cells per 100 μm length of the vessel, by analysis of frame-by-frame of video replay using Icy bioimage analysis software (http://icy.bioimageanalysis.org/). The values obtained from analyzing all recorded vessel segments, were averaged among groups of animals for statistical analysis. The percentages of vessels with normal blood flow, slow blood flow, and occluded vessels were calculated by dividing the number of each: vessels (both small and large) with normal blood flow, slow blood flow, and no blood flow by the total number of vessels recorded in all animals.

In-vivo snoRNA knockdown. For knockdown experiments, locked nucleic acid (LNA)-modified ASOs to specifically target snoRNAs or to target GFP (SEQ ID NOs: 44-48) as a control were injected IP every other day for three doses (2.5 mg/kg of total LNA per injection). Mouse Groups:6 sickle mice injected with antisense oligos U34, and U35; 6 sickle mice injected with antisense oligos U32, U34, and U35; 6 sickle mice injected with GFP-ASO.

Statistical analysis. Data were compared using parametric analyses (GraphPad Prism 9 Software, San Diego, CA), including repeated and non-repeated measures of analysis of variance (ANOVA). One-way and two-way ANOVA analyses were followed by Bonferroni corrections for multiple comparisons (multiplying the p value by the number of comparisons). A p value<0.05 was considered significant.

### Results

Rpl13a snoRNAs in Sickle RBCs Regulate ROS Production

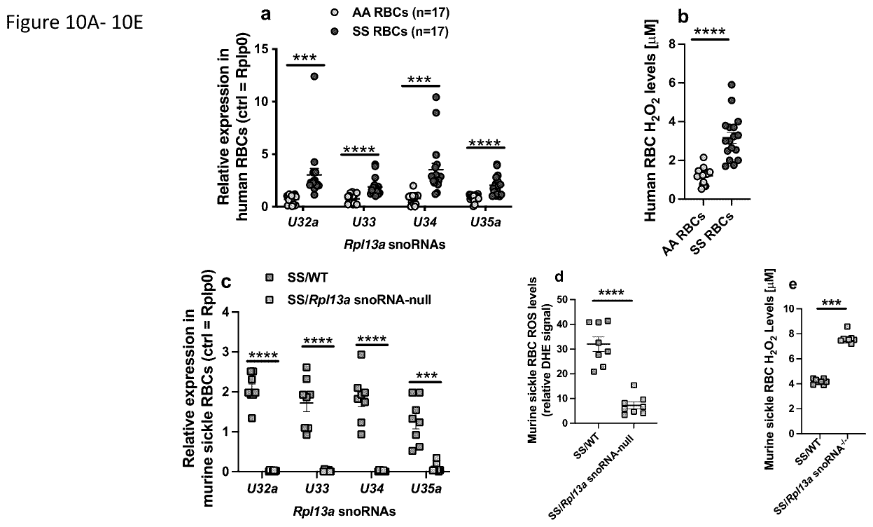

We first determined whether Rpl13a snoRNAs are present in human RBCs. We found that U32a, U33, U34, and U35a introns were present in normal (AA) and sickle (SS) RBCs (FIG. 10a). Notably, U32a, U33, U34, and U35a (p<0.001; FIG. 10a), as well as H₂O₂ (p<0.0001; FIG. 10b) were consistently and significantly more abundant in SS vs. AA RBCs, implying that human RBCs maintain cytosolic snoRNAs similar to how they maintain miRNAs³⁷, and that Rpl13a snoRNAs might be associated with ROS production and oxidative stress in SS RBCs.

To determine whether Rpl13a snoRNAs in sickle RBCs are directly involved in ROS production, we used SS/WT, and SS/Rpl13a snoRNA-null mice. We first compared the health of SS/Rpl13a snoRNA-null mice to the wild-type mice in which the mouse beta-globin locus has been replaced with an insert for human beta-globin (AA/WT). The SS/Rpl13a snoRNA-null animals remained for more than two years as healthy as the phenotypically normal AA/WT mice, until the mice were euthanized. Sickle RBCs from SS/Rpl13a snoRNA-null mice showed a significant decrease in U32, U33, U34, and U35a expression (p<0.0001; FIG. 10c), and lower ROS, but higher H₂O₂ levels (p<0.01; FIGS. 10d-e) compared to the SS/WT mice, suggesting that Rpl13a snoRNAs regulate ROS and H₂O₂ production in sickle RBCs.

Rpl13a snoRNAs Regulate Nox4 Expression in Sickle RBCs

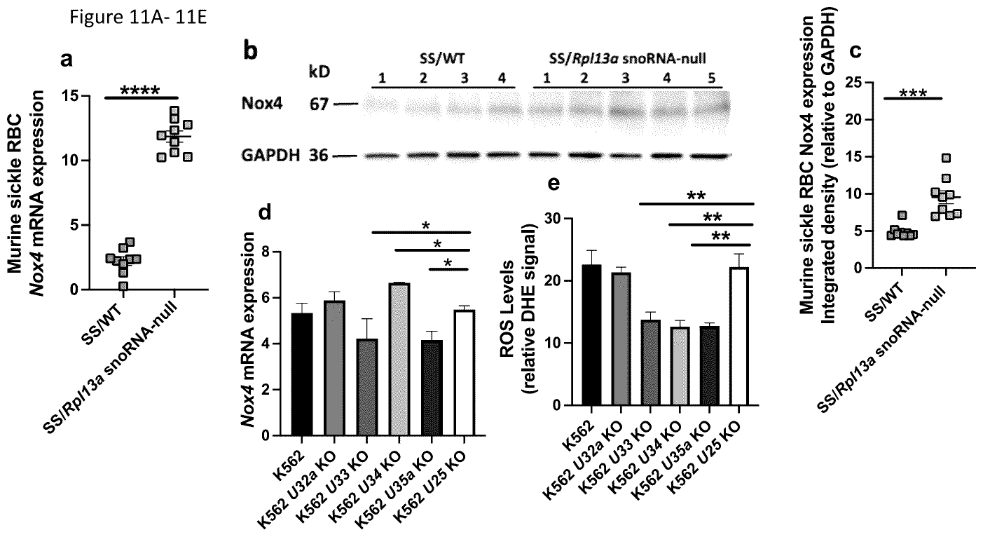

Because Rpl13a snoRNA depletion increased H₂O₂ generation in sickle RBCs, and since the major detected product of the constitutively active Nox4 is H₂O₂, we determined whether Rpl13a snoRNAs regulate H₂O₂ levels by affecting the transcription or translation of Nox4. Knocking-out Rpl13a snoRNAs in sickle mice increased sickle RBC Nox4 mRNA levels (p<0.0001; FIG. 11a), which was accompanied with an increase in Nox4 protein expression (p<0.001; FIG. 11b-c), suggesting that Rpl13a snoRNAs may affect Nox4 mRNA abundance in the cytosol of sickle RBCs.

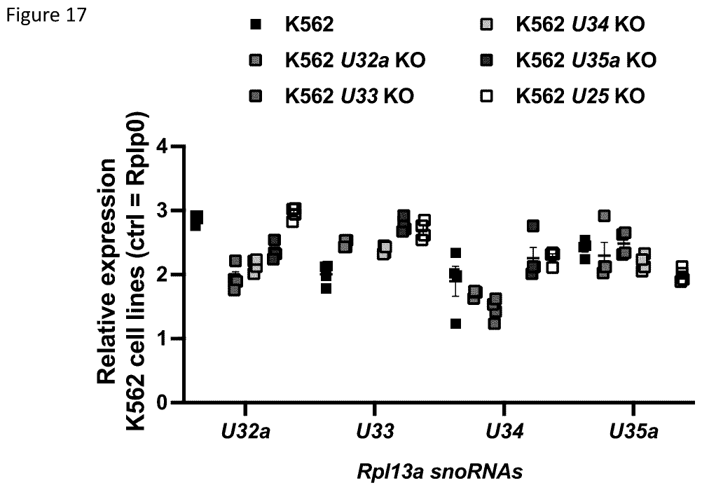

We then determined which Rpl13a snoRNAs are involved in affecting Nox4 mRNA expression, using the wild-type K562 and K562 KO mutants for each of the Rpl13a snoRNAs (U32a KO, U33 KO, U34 KO, U35a KO) (FIG. 17). A K562 KO for U25 encoded within an intron of a gene called ‘U22 host gene’ (UHG), was used as a control (FIG. 17), and all knock-out strains were generated by CRISPR-Cas9 targeted genome editing. Overall, there was no change in Nox4 mRNA expression, or ROS production in the WT control relative to K562 U25 KO cells (FIG. 11d-e). Compared to K562 U25 KO strain, U34 loss increased Nox4 mRNA expression (p<0.05), whereas loss of U33 or U35a markedly decreased Nox4 mRNA levels (p<0.05; FIG. 11d). Loss of any of the U33, U34, and U35a snoRNAs significantly lowered ROS levels compared to the K562 U25 KO strain (p<0.05; FIG. 11e). Knocking-out U32a however, failed to affect ROS levels. Together, these data indicate that U33, U34, and U35a, but not U32a, differentially affect Nox4 mRNA expression to dampen ROS generation.

U33, U34, and U35a Assemble into RNA-HuR Complexes in SS RBCs to Regulate Nox4 mRNA Stability

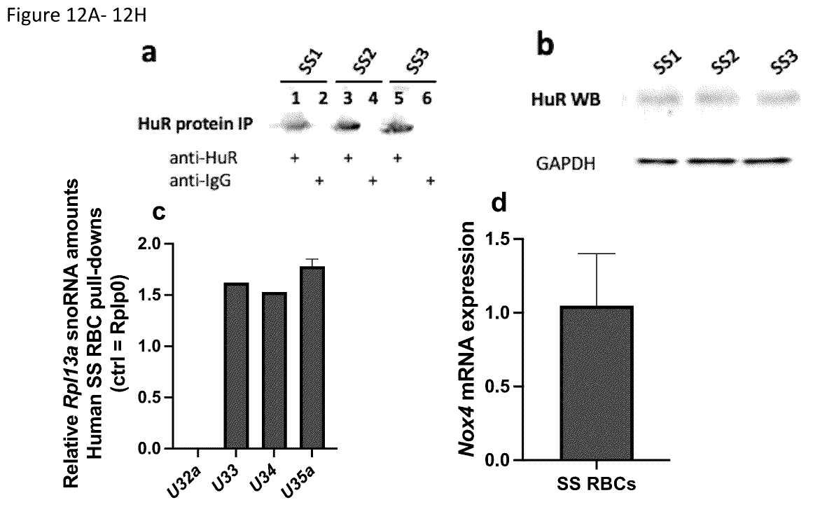

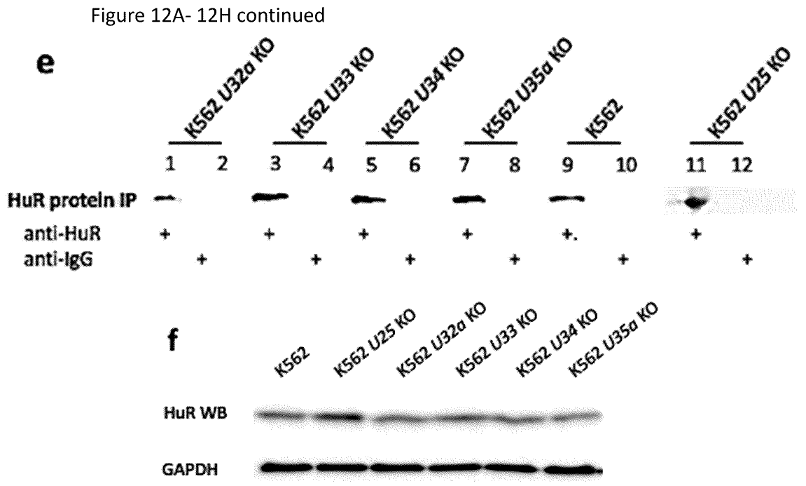

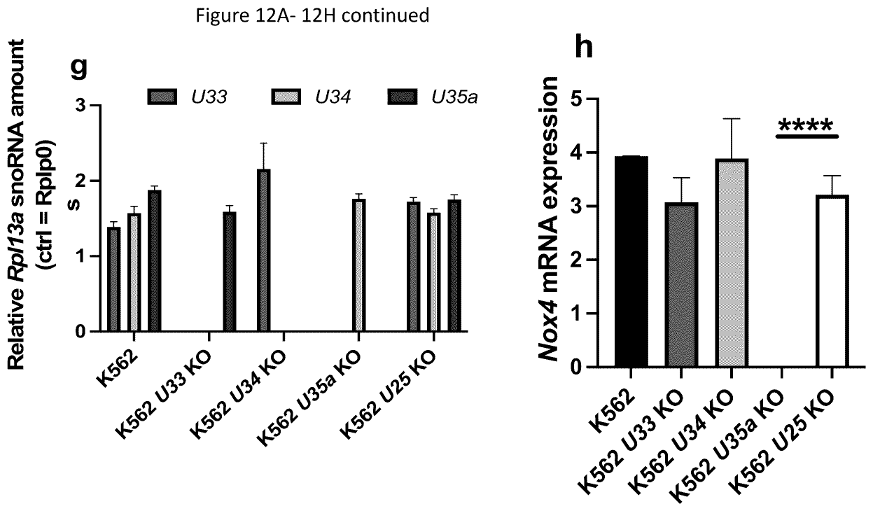

We subsequently hypothesized that U33, U34, and/or U35a may interact with a unique complement of cytosolic proteins to regulate Nox4 mRNA abundance in SCD. One of the most well-characterized RNA-binding proteins is human antigen R (HuR; ELAVL1), which increases mRNA stability by binding to the 3′ untranslated region (3′UTR) of the adenylate/uridylate (AU)-rich elements (AREs)³⁸. Because Nox4 mRNA carries several AREs in their 3′UTR³⁸, we suspected that HuR might be involved in snoRNA-mediated Nox4 mRNA regulation. To test whether Rpl13a snoRNAs interact with HuR to affect Nox4 mRNA expression, we pulled-down HuR from human SS RBCs using anti-HuR antibody (IgG1). HuR was successfully recovered in pull-downs performed with anti-HuR antibody (FIG. 12a-b), but not in the pull-downs performed with the control anti-IgG1 antibody (FIG. 12a). Immunoprecipitation (IP)-RT-qPCR analysis showed an enrichment of U33, U34 and U35a in the pull-downs; U32a was not detected (FIG. 12c). IP-RT-qPCR analysis additionally detected Nox4 mRNA in the pull-downs (FIG. 12d). These findings suggest that U33, U34 and U35a assemble into complexes with HuR, not previously described as a protein interacting with the box C/D snoRNAs, to affect Nox4 mRNA abundance and, presumably, translation efficiency.

We confirmed that U33, U34 and U35a form complexes with HuR to regulate Nox4 mRNA abundance, using our K562 Rpl13a snoRNA KO mutants. HuR was successfully pulled-down with anti-HuR antibody from all, K562 WT and KO mutants (FIG. 12e-f); but not with the anti-IgG1 antibody (FIG. 12e). HuR was co-immunoprecipitated with U33, U34 and U35a in the pull-downs prepared from the K562 WT and the control K562 U25 KO strain (FIG. 12g). However, the pull-downs prepared from the K562 U33 KO, K562 U34 KO, and K562 U35a KO strains, each carried only one Rpl13a snoRNA (U35a, U33, and U34, respectively; FIG. 12g). Nox4 mRNA was recovered from K562 WT, K562 U33 KO, K562 U34 KO, and K562 U25 KO strains (FIG. 12h), but not from K562 U35a KO cells (FIG. 12h). These data suggest that U33 and U35a assemble into complexes with Nox4 mRNA-HuR in sickle RBCs to increase or maintain the stability of Nox4 mRNA. Conversely, U34 appears to prevent HuR from complexing with and stabilizing Nox4 mRNA, likely leading to mRNA decay. Increased Nox4 mRNA stability and translation would ultimately lead to a decrease in ROS levels.

Sickle RBC Rpl13a snoRNAs Guide Peroxiredoxin2 mRNA 2′-O-Methylation, Leading to Inhibition of Translation In Vivo

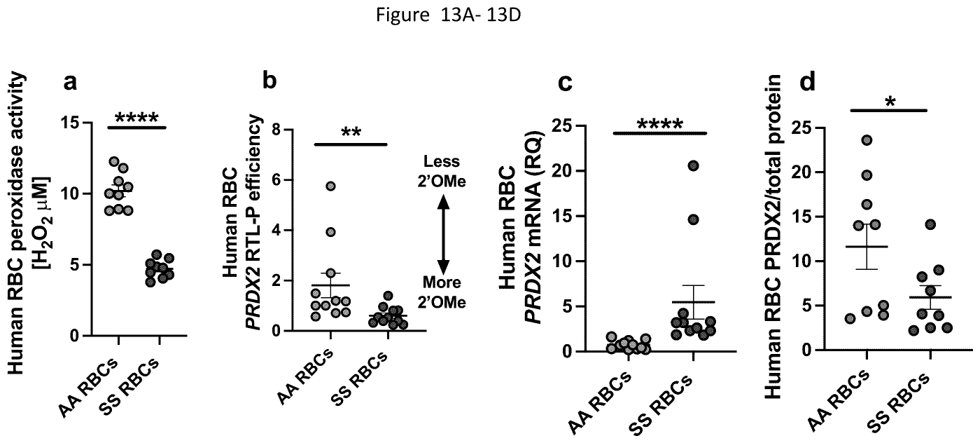

Additionally to the effects of Rpl13a snoRNA-mediated regulation of ROS and H₂O₂ production, we also determined the activity of peroxidases in both human and murine sickle RBCs depleted or not in snoRNAs to further understand the increased RBC H₂O₂ by Rpl13a snoRNA KO. Peroxidases consume H₂O₂, transferring electrons to acceptor proteins or generating other forms of molecular ROS that have much shorter half-lives than H₂O₂, such that peroxidase activity can lower the steady-state pool of both ROS and H₂O₂. Peroxidase activity was lower in human SS vs. AA RBCs (p<0.0001; FIG. 13a), suggesting that ROS- and Nox4-derived H₂O₂ accumulation in human SS RBCs is likely also due to additional regulation of the activity of peroxidases; enzymes protectors against oxidative damage.

The Rpl13a snoRNA U32a in 293T cells and the heart, has been evidenced to guide mRNA 2′-O-methylation of peroxidasin²⁹, a heme peroxidase mostly present in the vasculature, heart, and circulation^{39, 40}. However, since like peroxidasin, peroxiredoxin2 (Prdx2) is also a heme peroxidase, and because this thiol-dependent noncatalytic H₂O₂ scavenger⁴¹ is the third most abundant redox protein (15 million copies/cell) in RBCs⁴², we speculated that Prdx2 mRNA may undergo increased 2′-O-methylation in SS RBCs. Using RTL-P as a measure of 2′-O-methylation^{29, 35}, we found that Prdx2 mRNA was significantly more 2′-O-methylated in human SS relative to AA RBCs (p<0.01; FIG. 13b). RT-qPCR revealed constitutively higher Prdx2 mRNA levels in SS cells (p<0.0001; FIG. 13c) as well, but significantly lower Prdx2 protein levels than in AA RBCs (p<0.05; FIG. 13d). We conclude that low peroxidase activity in SS RBCs (FIG. 13a) is likely partly due to increased post-transcriptional 2′-O-methylation of Prdx2 mRNA, which subsequently reduces translation of the mRNA.

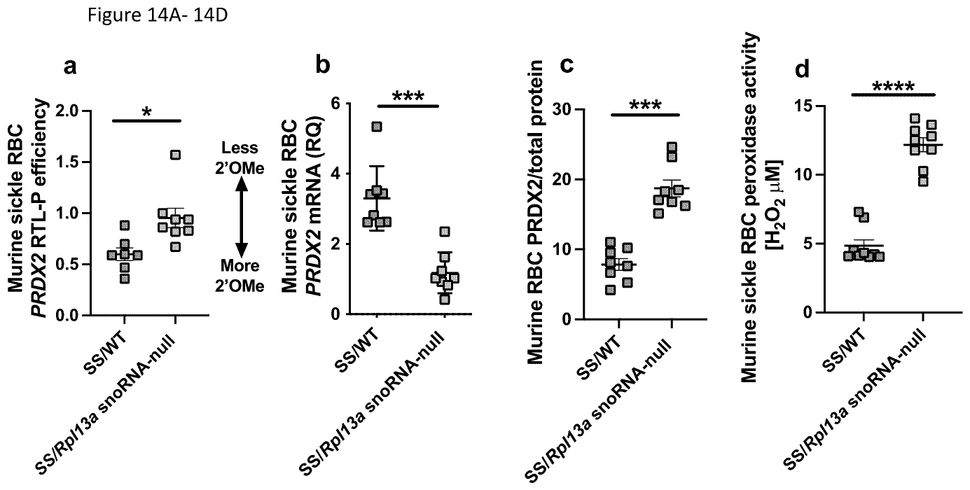

We next tested whether Rpl13a snoRNAs guide 2′-O-methylation of Prdx2 transcripts using sickle RBCs from our SS/WT and SS/Rpl13a snoRNA-null mice. RTL-P efficiency for Prdx2 mRNA was lower in sickle RBCs isolated from SS/Rpl13a snoRNA-null compared to SS/WT mice, consistent with the in vivo loss of 2′-O-methylation (p<0.05; FIG. 14a). Rpl13a snoRNA loss also significantly lowered Prdx2 mRNA levels in sickle RBCs relative to SS/WT controls (p<0.001; FIG. 14b), but Prdx2 protein expression and peroxidase activity were significantly higher (p<0.001; FIGS. 14c-d). Overall, these data suggest that Rpl3a snoRNAs guide Prdx2 mRNA 2′-O-methylation, consequently dampening its translation in the progenitor cells, leading to decreased protein levels and thereby cellular peroxidase activity in circulating sickle RBCs. Increased mRNA 2′-O-methylation may subsequently impact gene expression to regulate Prdx2 mRNA abundance during erythroid differentiation.

Prdx2 mRNA 2′-O-Methylation Requires U32a and U34

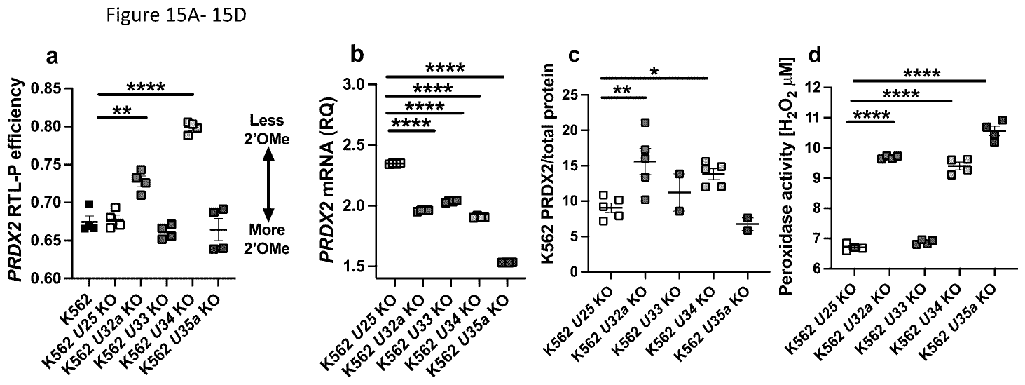

To implicate specific snoRNA(s) in the 2′-O-methylation of Prdx2 mRNA, we used K562 Rpl13a snoRNA KO mutants. Based on RTL-P efficiency, and while the control K562 U25 KO strain showed comparable Prdx2 mRNA 2′-O-methylation relative to the wild-type K562, knocking-out U32a or U34 in K562 diminished Prdx2 mRNA 2′-O-methylation compared to the K562 U25 KO cells (p<0.01; FIG. 15a). Prdx2 mRNA levels were lower in all K562 Rpl13a snoRNA KO mutants vs. K562 U25 KO cells (p<0.0001; FIG. 15b), but Prdx2 protein levels increased only in the two K562 cell lines with decreased mRNA 2′-O-methylation (i.e., U32a KO and U34 KO; p<0.05; FIG. 15c). However, peroxidase activity was increased not only in the U32a KO and U34 KO mutants but also in U35a KO cells relative to the controls (p<0.0001; FIG. 15d). These data suggest that U32a and U34 guide Prdx2 mRNA 2′-O-methylation, subsequently blunting the mRNA translation and associated peroxidase activity. U33 and U35a appear to have beneficial and detrimental effects, respectively, on peroxidase activity, even if they are not directly involved in Prdx2 mRNA 2′-O-methylation.

### Knocking-Down U34 and U35a Reduces Cell Adhesion, and Vaso-Occlusion Triggered by an Inflammatory Insult in Sickle Mice In Vivo

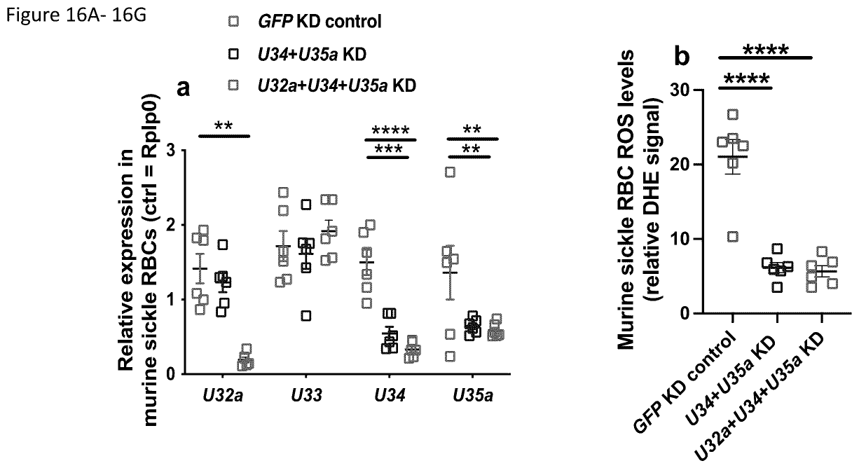

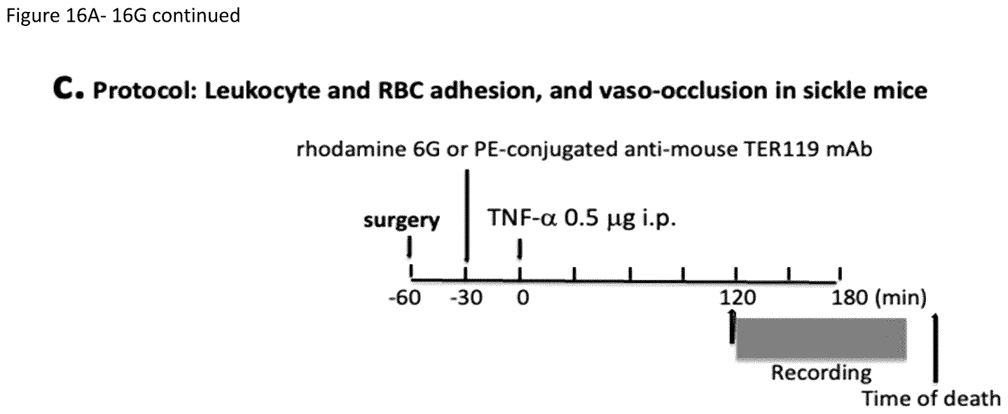

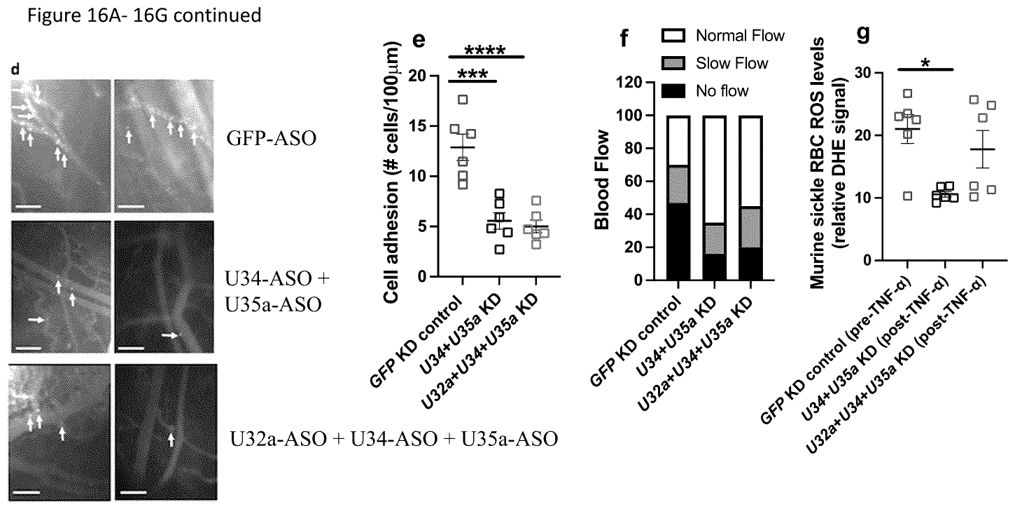

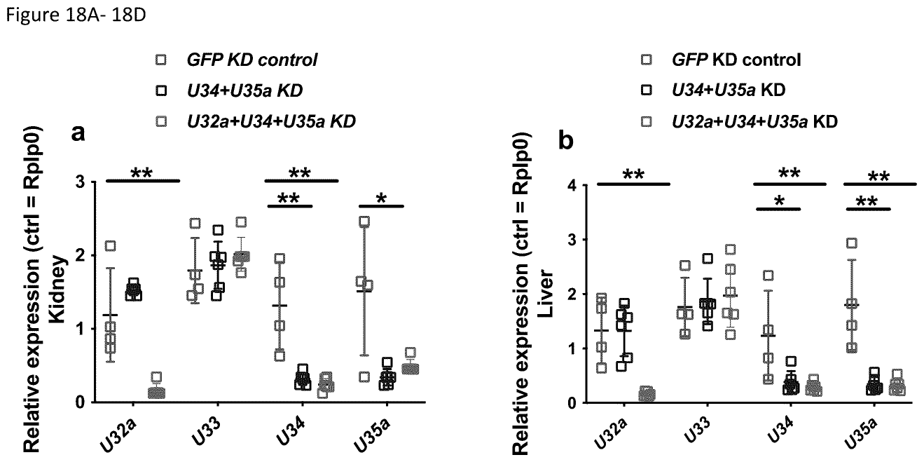

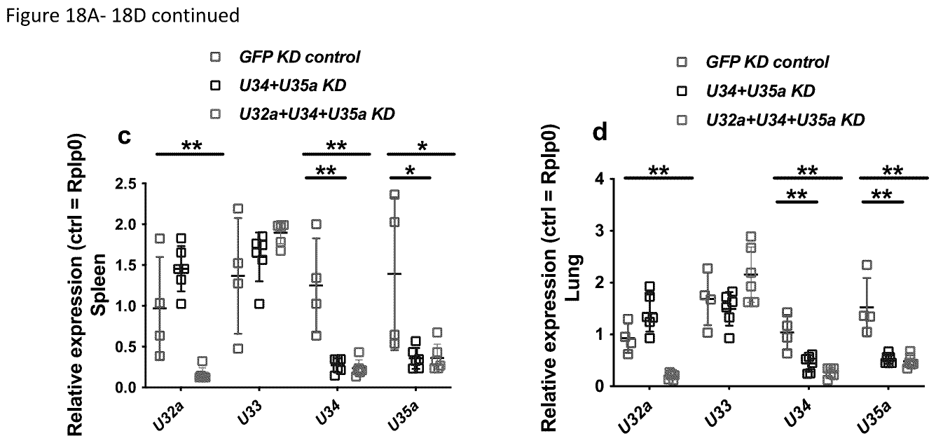

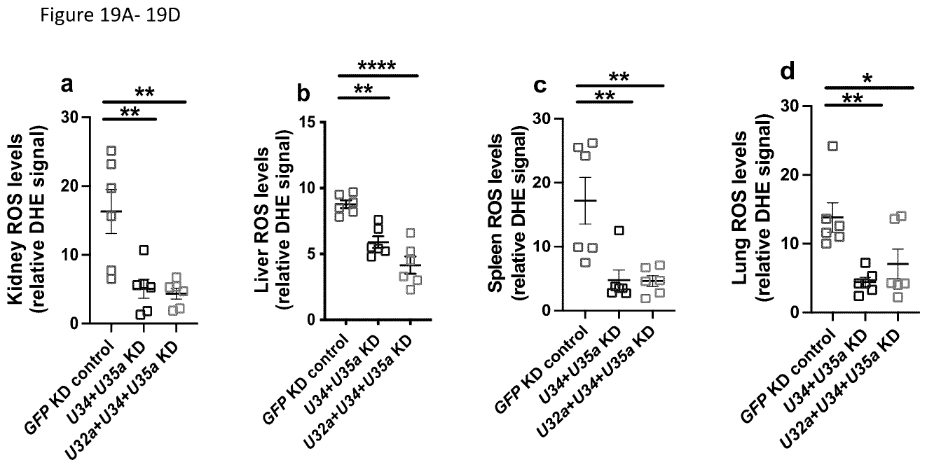

Based on our evidence that Rpl13a snoRNAs contribute to oxidative stress in SCD by both regulating Nox4 expression and repressing peroxidase activity, we assessed whether Rpl13a snoRNA depletion might have therapeutic benefits by decreasing the severity of vaso-occlusive crises. Since U34 and U35a were most responsible for upregulating ROS in sickle RBCs (FIGS. 12 and 15), in vivo knocking-down U34 and U35a (U34+U35a KD) in sickle mice with U34-ASO and U35a-ASO significantly down-regulated U34 and U35a expression in sickle RBCs (p<0.01; FIG. 16a), and lowered sickle RBC ROS generation by 71% compared to sickle mice injected with the control GFP-ASO (p<0.0001; FIG. 16b). U34+U35a KD sickle mice also showed reduced U34 and U35a expression in the kidneys (p<0.05), liver (p<0.05), spleen (p<0.05), and lungs (p<0.01; FIG. 18a-d), which led to blunt organ ROS levels (p<0.01; FIG. 19a-d) compared to the GFP KD controls.

By intravital microscopy, we monitored cell adhesion and vaso-occlusion in U34+U35a KD mice for 120 minutes, starting 120 minutes post TNF-α injection (FIG. 16c), which provides enough time for leukocytes to be recruited and adhere to the endothelium, for sickle RBCs to bind to adherent leukocytes and the endothelium, and for vaso-occlusive-like processes to occur in the micro-vessels. The GFP-ASO-treated controls showed extensive interactions between RBCs, activated leukocytes, and the microvascular endothelium following TNF-α injection, leading to transient or permanent vaso-occlusion as evidenced by blood stasis (FIG. 16d). These effects were sustained for more than 180 minutes post-TNF-α administration. In contrast, the U34+U35a KD mice showed reduced cell adhesion (FIG. 16d), with the number of adherent cells over 100 μm venular lengths decreasing by 57% compared to the GFP-ASO group (p<0.001; FIG. 16e). As a result of inhibition of cell adhesion, blood stasis was reduced (FIG. 16d-e, f). Only 16% of the total vessels we recorded showed blood stasis in U34+U35a KD sickle mice, compared to 47% in the GFP-ASO group (FIG. 16f). TNF-α-induced sickle RBC ROS levels were also lowered by 50% in U34+U35a KD sickle mice vs. the GFP-ASO control group before being injected with TNF-α (p<0.05; FIG. 16g). These data suggest that silencing U34 and U35a reduces the intensity of vaso-occlusion triggered by TNF-α, by preventing sickle RBCs and activated leukocytes from interacting with enflamed vessels and/or sickle RBCs from binding to adherent leukocytes.

U32a was also involved in decreasing peroxidase activity and thus increasing oxidative stress in sickle RBCs (FIG. 15). We determined whether silencing U32a in addition to U34 and U35a is superior against TNF-α-triggered vaso-occlusion than silencing U34 and U35a alone. Relative to GFP-ASO-treated animals, in vivo triple-KD of U32a, U34 and U35a significantly reduced U32a, U34, and U35a expression in sickle RBCs tested prior to TNF-α injection to U32a+U34+U35a KD sickle mice (p<0.01; FIG. 16a), with 73% reduction in ROS generation (p<0.0001; FIG. 16b). U32a, U34 and/or U35a expression also declined in the kidneys (p<0.05), liver (p<0.01), spleen (p<0.05), and lungs (p<0.01) in U32a+U34+U35a KD sickle mice (FIG. 18a-d), which led to a much lower organ ROS levels (p<0.05; FIG. 19a-d) compared to the controls.

Importantly, U32a+U34+U35a KD sickle mice injected with TNF-α showed 61% reduction in cell adhesion in enflamed vessels (p<0.0001; FIG. 16d-e). This led to normal Blood flow in 55% of the total vessels we observed in U32a+U34+U35a KD sickle mice compared to only 30% of the venules with normal blood flow in the control group (FIG. 16f). But, this beneficial effect in the triple-KD animals was not superior to that seen in the U34+U35a KD animals, since blood flow was sustained in only 65% of the vessels we recorded. The triple-KD treatment also failed to reduce sickle RBC ROS profile following TNF-α injection (p>0.05; FIG. 16g). Thus, while silencing U32a, U34 and U35a effectively abrogates cell adhesion and vaso-occlusion in sickle mice in response to TNF-α, knocking-down U32a additionally to U34 and U35a does not have additional therapeutic benefits relative to silencing U34 and U35a only.

### Discussion

Our data show that Rpl3a snoRNAs remain present at high abundances in human SS RBCs, and they are associated with increased ROS levels and oxidative stress-induced vaso-occlusion in vivo. These sickle cell snoRNAs guide HuR-mediated control of Nox4 mRNA stability and subsequently translation, and post-transcriptional Prdx2 mRNA 2′-O-methylation. Importantly, our data show that genetic targeting of the Rpl13a snoRNAs U34 and U35a prevents oxidative stress from promoting vaso-occlusive pathology in SCD.

ROS produced by Nox enzymes^{3, 43} and the ensuing oxidative stress in sickle RBCs mediates adhesion to the endothelium in vitro and in vivo^{2, 3}. Our pull-down assays showed that sickle RBC Rpl13a snoRNAs U33, U34, and U35a regulate ROS and H₂O₂ levels in vivo by assembling into complexes with cytoplasmic HuR to target Nox4 mRNA, thus regulating transcript abundance and presumably translation efficiency. Specifically, U34 appeared to promote HuR-dependent Nox4 mRNA decay, whereas U33 and U35a formed complexes with HuR to either increase or maintain Nox4 mRNA stability. To our knowledge, this is the first description of interactions between snoRNAs, HuR, and Nox4 mRNA within the same cellular compartment. Because HuR binds to AREs in the 3′UTR of mRNA to regulate translation, we suspect that U33, U34, and U35a modulate the stability of Nox4 mRNA by directing HuR to bind to Nox4 mRNA possibly in a sequence-specific manner. While several proteins that are known to associate with the Rpl13a snoRNAs might be present in sickle RBCs, including fibrillarin and 15.5K²⁰-2³, proteins that associate in the cytosol under homeostatic conditions, like HuR, suggest that such snoRNPs are also dynamic. To date, there is no data linking oxidative stress to the regulation of Nox4 transcript abundance by Rpl13a snoRNAs through AREs. We further demonstrated that the Rpl13a snoRNAs U32a and U34 have a complementary effect on ROS accumulation in sickle RBCs by guiding the 2′-O-methylation of Prdx2 mRNA to inhibit its translation and thereby reduce peroxidase activity. We suspect that this methylation is added dynamically to mature mRNA at one or more sites, as 2′-O-methylated Prdx2 mRNA was detected in the enucleated sickle cells. Such post-transcriptional modification might subsequently affect gene expression to thereby regulate the mRNA levels. Our collective results specifically implicate a dual role for sickle RBC U34, forming complexes with HuR to promote Nox4 mRNA decay and guiding Prdx2 mRNA epitranscriptomic modification in vivo.

Importantly, silencing U34 and U35a with their respective ASO in sickle mice reduced ROS-induced sickle RBC adhesion and vaso-occlusion, and restored blood flow in vivo. These positive therapeutic outcomes were at least partially due to the marked decrease in sickle RBC ROS production and oxidative damage. Sickle RBC adhesion is a key component of vaso-occlusion, vascular damage, and inflammation in SCD patients, and by studying the mechanisms of action of Rpl13a snoRNAs in sickle RBCs, we were able to deduce that the aberrant accumulation of U34 and U35a in sickle RBCs contributes to the deleterious ROS accumulation-triggered vaso-occlusion in sickle mice. Further, we speculated that Rpl13a snoRNAs in endothelial cells, and likely leukocytes may also be implicated in driving acute vaso-occlusive events in SCD, and that U34+U35a ASO treatment may protect against vaso-occlusion by acting not only on sickle RBCs, but on the vascular endothelium and possibly leukocytes as well. Endothelial oxidative stress caused by Nox-derived superoxide triggers vaso-occlusion in SCD⁴⁴, and silencing U34 and U35a in sickle mice lowered endothelial ROS. Leukocytes, especially neutrophils, like sickle RBCs, have an important role in mediating vaso-occlusion by interacting with the vascular endothelium and sickle RBCs^{45, 46}, and Nox is a major source of ROS production in activated leukocytes^{45, 46}. Thus, the U34+U35a ASO treatment in addition to its effect on sickle RBCs, may therefore reduce leukocyte adhesion and endothelial dysfunction as well via targeting Nox enzymes, and could thus represent a novel therapeutic intervention for preventing acute pain crises in SCD.

Given the role of Rpl13a snoRNAs in sickle RBCs, identification of additional targets for each of the Rpl13a snoRNAs will be a complex task and the subject of future investigation. Nonetheless, for these over-expressed small ncRNAs, the approach consisting of using ASOs appears to be effective in attenuating or blocking the effects of the ncRNAs in a SCD mouse model. Early-phase human clinical trials that target miRNAs raise a lot of trust in the feasibility of applying silencing Rpl13a snoRNA strategies to modulate their level and function as a therapy to prevent pain crises in SCD patients.

### REFERENCES

1. Aslan M, Thornley-Brown D and Freeman B A. Reactive species in sickle cell disease. Annals of the New York Academy of Sciences. 2000; 899:375-91.
2. Thamilarasan M, Estupinan R, Batinic-Haberle I and Zennadi R. Mn porphyrins as a novel treatment targeting sickle cell NOXs to reverse and prevent acute vaso-occlusion in vivo. Blood Adv. 2020; 4:2372-2386.
3. MacKinney A, Woska E, Spasojevic I, Batinic-Haberle I and Zennadi R. Disrupting the vicious cycle created by NOX activation in sickle erythrocytes exposed to hypoxia/reoxygenation prevents adhesion and vasoocclusion. Redox biology. 2019:101097.
4. Mahaseth H, Vercellotti G M, Welch T E, Bowlin P R, Sonbol K M, Hsia C J, Li M, Bischof J C, Hebbel R P and Belcher J D. Polynitroxyl albumin inhibits inflammation and vasoocclusion in transgenic sickle mice. The Journal of laboratory and clinical medicine. 2005; 145:204-11.
5. George A, Pushkaran S, Li L, An X, Zheng Y, Mohandas N, Joiner C H and Kalfa T A. Altered phosphorylation of cytoskeleton proteins in sickle red blood cells: the role of protein kinase C, Rac GTPases, and reactive oxygen species. Blood Cells Mol Dis. 2010; 45:41-5.
6. Jagadeeswaran R, Vazquez B A, Thiruppathi M, Ganesh B B, Ibanez V, Cui S, Engel J D, Diamond A M, Molokie R E, DeSimone J, Lavelle D and Rivers A. Pharmacological inhibition of LSD1 and mTOR reduces mitochondrial retention and associated ROS levels in the red blood cells of sickle cell disease. Experimental hematology. 2017; 50:46-52.
7. Nader E, Grau M, Fort R, Collins B, Cannas G, Gauthier A, Walpurgis K, Martin C, Bloch W, Poutrel S, Hot A, Renoux C, Thevis M, Joly P, Romana M, Guillot N and Connes P. Hydroxyurea therapy modulates sickle cell anemia red blood cell physiology: Impact on RBC deformability, oxidative stress, nitrite levels and nitric oxide synthase signalling pathway. Nitric oxide: biology and chemistry official journal of the Nitric Oxide Society. 2018; 81:28-35.
8. Mohandas N and Evans E. Sickle erythrocyte adherence to vascular endothelium. Morphologic correlates and the requirement for divalent cations and collagen-binding plasma proteins. J Clin Invest. 1985; 76:1605-12.
9. Hofstra T C, Kalra V K, Meiselman H J and Coates T D. Sickle erythrocytes adhere to polymorphonuclear neutrophils and activate the neutrophil respiratory burst. Blood. 1996; 87:4440-7.
10. Zennadi R, Chien A, Xu K, Batchvarova M and Telen M J. Sickle red cells induce adhesion of lymphocytes and monocytes to endothelium. Blood. 2008; 112:3474-83.
11. Kaul D K, Liu X D, Zhang X, Ma L, Hsia C J and Nagel R L. Inhibition of sickle red cell adhesion and vasoocclusion in the microcirculation by antioxidants. American journal of physiology Heart and circulatory physiology. 2006; 291: H167-75.
12. Bernaudin F, Verlhac S, Chevret S, Torres M, Coic L, Arnaud C, Kamdem A, Hau I, Grazia Neonato M and Delacourt C. G6P D deficiency, absence of alpha-thalassemia, and hemolytic rate at baseline are significant independent risk factors for abnormally high cerebral velocities in patients with sickle cell anemia. Blood. 2008; 112:4314-7.
13. Sachdev V, Kato G J, Gibbs J S, Barst R J, Machado R F, Nouraie M, Hassell K L, Little J A, Schraufnagel D E, Krishnamurti L, Novelli E M, Girgis R E, Morris C R, Rosenzweig E B, Badesch D B, Lanzkron S, Castro O L, Taylor J Gt, Hannoush H, Goldsmith J C, Gladwin M T and Gordeuk V R. Echocardiographic markers of elevated pulmonary pressure and left ventricular diastolic dysfunction are associated with exercise intolerance in adults and adolescents with homozygous sickle cell anemia in the United States and United Kingdom. Circulation. 2011; 124:1452-60.
14. Balkaran B, Char G, Morris J S, Thomas P W, Serjeant B E and Serjeant G R. Stroke in a cohort of patients with homozygous sickle cell disease. The Journal of pediatrics. 1992; 120:360-6.
15. Hebbel R P. Beyond hemoglobin polymerization: the red blood cell membrane and sickle disease pathophysiology. Blood. 1991; 77:214-37.
16. Hebbel R P, Boogaerts M A, Eaton J W and Steinberg M H. Erythrocyte adherence to endothelium in sickle-cell anemia. A possible determinant of disease severity. N Engl J Med. 1980; 302:992-5.
17. Powars D R, Elliott-Mills D D, Chan L, Niland J, Hiti A L, Opas L M and Johnson C. Chronic renal failure in sickle cell disease: risk factors, clinical course, and mortality. Annals of internal medicine. 1991; 115:614-20.
18. Nur E, Brandjes D P, Teerlink T, Otten H M, Oude Elferink R P, Muskiet F, Evers L M, ten Cate H, Biemond B J, Duits A J and Schnog J J. N-acetylcysteine reduces oxidative stress in sickle cell patients. Annals of hematology. 2012; 91:1097-105.
19. Michel C I, Holley C L, Scruggs B S, Sidhu R, Brookheart R T, Listenberger L L, Behlke M A, Ory D S and Schaffer J E. Small nucleolar RNAs U32a, U33, and U35a are critical mediators of metabolic stress. Cell Metab. 2011; 14:33-44.
20. Tyc K and Steitz J A. U3, U8 and U13 comprise a new class of mammalian snRNPs localized in the cell nucleolus. EMBO J. 1989; 8:3113-9.
21. Liu J and Maxwell E S. Mouse U14 snRNA is encoded in an intron of the mouse cognate hsc70 heat shock gene. Nucleic Acids Res. 1990; 18:6565-71.
22. Gautier T, Berges T, Tollervey D and Hurt E. Nucleolar KKE/D repeat proteins Nop56p and Nop58p interact with Nop1p and are required for ribosome biogenesis. Molecular and cellular biology. 1997; 17:7088-98.
23. Watkins N J, Segault V, Charpentier B, Nottrott S, Fabrizio P, Bachi A, Wilm M, Rosbash M, Branlant C and Luhrmann R. A common core RNP structure shared between the small nucleoar box C/D RNPs and the spliceosomal U4 snRNP. Cell. 2000; 103:457-66.
24. Holley C L and Topkara V K. An introduction to small non-coding RNAs: miRNA and snoRNA. Cardiovascular drugs and therapy sponsored by the International Society of Cardiovascular Pharmacotherapy. 2011; 25:151-9.
25. Tollervey D and Kiss T. Function and synthesis of small nucleolar RNAs. Curr Opin Cell Biol. 1997; 9:337-42.
26. Silva D G, Belini Junior E, Torres Lde S, Ricci Junior O, Lobo Cde C, Bonini-Domingos C R and de Almeida E A. Relationship between oxidative stress, glutathione S-transferase polymorphisms and hydroxyurea treatment in sickle cell anemia. Blood Cells Mol Dis. 2011; 47:23-8.
27. Wood K C and Granger D N. Sickle cell disease: role of reactive oxygen and nitrogen metabolites. Clin Exp Pharmacol Physiol. 2007; 34:926-32.
28. Takac I, Schroder K, Zhang L, Lardy B, Anilkumar N, Lambeth J D, Shah A M, Morel F and Brandes R P. The E-loop is involved in hydrogen peroxide formation by the NADPH oxidase Nox4. J Biol Chem. 2011; 286:13304-13.
29. Elliott B A, Ho H T, Ranganathan S V, Vangaveti S, Ilkayeva O, Abou Assi H, Choi A K, Agris P F and Holley C L. Modification of messenger RNA by 2-O-methylation regulates gene expression in vivo. Nat Commun. 2019; 10:3401.
30. Levasseur D N, Ryan T M, Pawlik K M and Townes T M. Correction of a mouse model of sickle cell disease: lentiviral/antisickling beta-globin gene transduction of unmobilized, purified hematopoietic stem cells. Blood. 2003; 102:4312-9.
31. Townes T M, Ryan T M, Behringer R R, Palmiter R D and Brinster R L. DNase I super-hypersensitive sites direct high level erythroid expression of human alpha-, beta- and beta s-globin genes in transgenic mice. Progress in clinical and biological research. 1989; 316A:47-61.
32. Ryan T M, Townes T M, Reilly M P, Asakura T, Palmiter R D, Brinster R L and Behringer R R. Human sickle hemoglobin in transgenic mice. Science. 1990; 247:566-8.
33. Chantrathammachart P, Mackman N, Sparkenbaugh E, Wang J G, Parise L V, Kirchhofer D, Key N S and Pawlinski R. Tissue factor promotes activation of coagulation and inflammation in a mouse model of sickle cell disease. Blood. 2012; 120:636-46.
34. Lee J, Harris A N, Holley C L, Mahadevan J, Pyles K D, Lavagnino Z, Scherrer D E, Fujiwara H, Sidhu R, Zhang J, Huang S C, Piston D W, Remedi M S, Urano F, Ory D S and Schaffer J E. Rpl13a small nucleolar RNAs regulate systemic glucose metabolism. J Clin Invest. 2016; 126:4616-4625.
35. Zennadi R, Moeller B J, Whalen E J, Batchvarova M, Xu K, Shan S, Delahunty M, Dewhirst M W and Telen M J. Epinephrine-induced activation of L W-mediated sickle cell adhesion and vaso-occlusion in vivo. Blood. 2007; 110:2708-17.
36. Chen S Y, Wang Y, Telen M J and Chi J T. The genomic analysis of erythrocyte microRNA expression in sickle cell diseases. PloS one. 2008; 3:e2360.
37. Meisner N C and Filipowicz W. Properties of the Regulatory RNA-Binding Protein HuR and its Role in Controlling miRNA Repression. Adv Exp Med Biol. 2011; 700:106-23.
38. Cheng G, Salerno J C, Cao Z, Pagano P J and Lambeth J D. Identification and characterization of VPO1, a new animal heme-containing peroxidase. Free radical biology & medicine. 2008; 45:1682-94.
39. Cheng G, Li H, Cao Z, Qiu X, McCormick S, Thannickal V J and Nauseef W M. Vascular peroxidase-1 is rapidly secreted, circulates in plasma, and supports dityrosine cross-linking reactions. Free radical biology & medicine. 2011; 51:1445-53.
40. Nagababu E, Mohanty J G, Friedman J S and Rifkind J M. Role of peroxiredoxin-2 in protecting RBCs from hydrogen peroxide-induced oxidative stress. Free Radic Res. 2013; 47:164-71.
41. Low F M, Hampton M B, Peskin A V and Winterbourn C C. Peroxiredoxin 2 functions as a noncatalytic scavenger of low-level hydrogen peroxide in the erythrocyte. Blood. 2007; 109:2611-7.
42. Dong Z W, Shao P, Diao L T, Zhou H, Yu C H and Qu L H. RTL-P: a sensitive approach for detecting sites of 2-O-methylation in RNA molecules. Nucleic Acids Res. 2012; 40:e157.
43. George A, Pushkaran S, Konstantinidis D G, Koochaki S, Malik P, Mohandas N, Zheng Y, Joiner C H and Kalfa T A. Erythrocyte NADPH oxidase activity modulated by Rac GTPases, PKC, and plasma cytokines contributes to oxidative stress in sickle cell disease. Blood. 2013; 121:2099-107.
44. Wood K C, Hebbel R P and Granger D N. Endothelial cell NADPH oxidase mediates the cerebral microvascular dysfunction in sickle cell transgenic mice. FASEB J. 2005; 19:989-91.
45. Paloschi M V, Boeno C N, Lopes J A, Eduardo Dos Santos da Rosa A, Pires W L, Pontes A S, da Silva Setubal S, Soares A M and Zuliani J P. Role of 1-amino acid oxidase isolated from Calloselasma rhodostoma venom on neutrophil NADPH oxidase complex activation. Toxicon. 2018; 145:48-55.
46. Turhan A, Weiss L A, Mohandas N, Coller B S and Frenette P S. Primary role for adherent leukocytes in sickle cell vascular occlusion: a new paradigm. Proc Natl Acad Sci USA. 2002; 99:3047-51.

### Example III—the Role for Rpl13a snoRNAs in Sickling and Regulating Genes Involved in γ-Globin Repression in Sickle Erythroid Progenitors

Sickle RBCs are prone to hemolysis due to increased oxidative stress, resulting in severe anemia, accompanied by stress erythropoiesis. In addition, in hypoxic conditions, RBCs undergo sickling promoting vaso-occlusion, which steers progressive vasculopathy, significant morbidity, and premature death. Hence, SCD treatment options are still limited. Current studies support the premise that fetal hemoglobin (HbF) is the most powerful natural inhibitor of SCD pathophysiology, and that reactivating the silenced fetal γ-globin gene would be therapeutic in SCD patients. The severity of SCD clinical symptoms are alleviated by reactivating γ-globin gene of fetal hemoglobin (HbF) expression. Reducing oxidative stress also ameliorates SCD phenotypic severity. Yet, treatment methods are severely impeded by a lack of regulatory mechanistic knowledge regarding γ-globin gene silencing and β-globin switching. We have now identified an unanticipated and unique mechanism of Rpl13a snoRNAs that provides unprecedented insights into regulation of fetal γ-globin gene in SCD, and the extent of their impact on sickling, and the associated events, and may point to a new targeted intervention against SCD sequelae.

Introduction and Rationale—Clinical manifestations associated with SCD vary in frequency and severity between patients due in part to variable HbF levels. Among different treatment strategies investigated, reactivation of γ-globin gene transcription to increase HbF production, remains the most effective one to ameliorate SCD clinical symptoms. At high enough levels, HbF directly influences HbS polymer stability, and inhibits polymerization of HbS, and subsequent sickling at low O₂ tension, and vaso-occlusion, producing clinical benefit. Thus, these findings urge for extensive investigations to define mechanisms of γ-globin regulation. HbF is a heterogeneous mixture of γ-globin polypeptide chains containing either glycine (^{G}γ) or alanine (^{A}γ) at residue 136. Infants with Hb SS have a delay in the γ- to β-globin switch, and HbF levels average 9% at 24 months of age. In theory, therapeutic γ-globin reactivation could be accomplished by either inhibition of repressor proteins to prevent silencing, or enforced expression of trans-activators. The transcription factor BCL11A is a major negative regulators of γ-globin gene transcription, and KLF1 transcription factor controls the fetal globin to adult switch by activating β-globin indirectly repressing γ-globin expression. Decreased, but not absent, KLF1 in human and mouse adult erythroid progenitors reduced BCL11A levels and increased human γ-globin/β-globin expression ratios³⁸. KLF1 binds to the BCL11A promoter. Further, BCL11A interacts with SOX6, the high-mobility group (HMG)-box containing transcription factor, co-occupy the β-globin cluster, and cooperate in silencing γ-globin transcription in adult human erythroblasts. Knockdown of SOX6 in human erythroblasts led to a small upregulation of HbF, but when combined with BCL11A knockdown, further enhanced HbF production, beyond that achieved with either knockdown alone. BCL11A is directly stimulated by ATF4, a heme-regulated inhibitor (HRI)-regulated protein of γ-globin regulator, by binding to its enhancer and promoting enhancer-promoter contact. BCL11A occupies several discrete regions within the human β-globin cluster, and acts within the β-globin locus. These regions include the third hypersensitive site (HS)3 of the locus control region (LCR) and an intergenic region between Aγ-globin and δ-globin genes. Thus, since these four transcription factors interact and regulate each other to functionally repress HbF gene, an understanding how this molecular circuitry controlling HbF is regulated, is a mandatory precondition for rationally designed therapies for SCD.

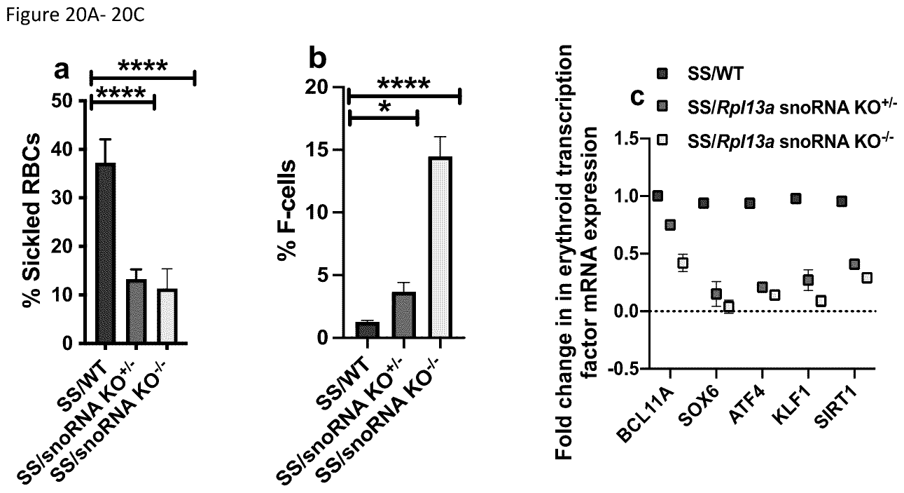

The Rpl13a snoRNAs are novel short non-coding RNAs regulators of ROS levels and oxidative stress response. Our data have shown that Rpl13a snoRNA loss in sickle RBCs reduced RBC ROS levels and oxidative stress. Our unexpected data show that Rpl13a snoRNAs in sickle erythroblasts regulate the expression of transcription factors silencers of γ-globin gene, and are implicated in RBC sickling (FIG. 20). We hypothesized that Rpl13a snoRNAs regulate γ-globin gene transcription in the developing erythroblast, and that depleting these cells of the snoRNAs up-regulates HbF levels, consequently reducing sickling of circulating RBCs at low O₂ tension, and extending RBC lifespan in SCD. We found that Rpl13a snoRNAs are involved in sickling, since their loss in SS/Rpl13a snoRNA^{+/-} and SS/Rpl13a snoRNA^{-/-} mice not only reduced sickling at 1% O₂ by 64% and 70%, respectively (p<0.01; FIG. 20a), but also increased % F-cells compared to SS/WT mice (p<0.01; FIG. 20b). Rpl13a snoRNA loss in erythroblasts generated from the bone marrow of SS/Rpl13a snoRNA^{+/-} and SS/Rpl13a snoRNA^{-/-} mice also decreased mRNA levels of the transcription factors KLF1, BCL11A, SOX6 and ATF4, silencers of γ-globin gene expression vs. SS/WT mice (p<0.01; FIG. 20c), suggesting that Rpl13a snoRNAs are likely involved in γ-globin gene silencing. Sickling is another driving force of hemolysis. Our data have shown that Rpl13a snoRNA loss in sickle mice increased RBC counts, Hb, hematocrit and mean corpuscular Hb (MCHC), while reticulocyte counts, which are associate with SCD severity, were blunted compared to SS/WT mice (FIG. 6A-I). Rpl13a snoRNA loss in sickle mice also reduced extramedullary hematopoiesis in the spleen (FIG. 5E-G). This suggests that Rpl13a snoRNAs are implicated in ineffective erythropoiesis in SCD. Thus, these findings reinforce our hypothesis by underscoring the pathogenic significance of snoRNAs in SCD, and Rpl13a snoRNAs implication in sickling by regulating genes involved in γ-globin repression in sickle erythroid progenitors.

1. A composition capable of reducing and/or inhibiting a Rpl13a snoRNA in a cell and/or subject, wherein the composition comprises an antisense oligonucleotide or a guide RNA specific for U34.

2. (canceled)

3. (canceled)

4. The composition of claim 1, wherein the composition further comprises an antisense oligonucleotide or a guide RNA specific for U35a.

5. (canceled)

6. The composition of claim 1, wherein the composition further comprises an antisense oligonucleotide or a guide RNA specific for U32a.

7. (canceled)

8. The composition of claim 1, wherein the composition further comprises an antisense oligonucleotide or a guide RNA specific for both U32a and U35a.

9. (canceled)

10. The composition of claim 1, wherein the composition comprises an antisense oligonucleotide, wherein the antisense oligonucleotides is complementary to at least a portion of the snoRNA.

11. The composition of claim 10, wherein the antisense oligonucleotide comprises at least one modified nucleotide.

12. The composition of claim 10, wherein the antisense oligonucleotide is at least 15 nucleotides long.

13. (canceled)

14. A pharmaceutical composition comprising the composition of claim 1, and a pharmaceutical acceptable excipient, carrier or diluent.

15. (canceled)

16. A method for treating and/or preventing hemoglobinopathies in a subject in need thereof, the method comprising administering a therapeutically effective amount of a composition capable of reducing and/or inhibiting a Rpl13a snoRNA in a subject or cell.

17. The method of claim 16, wherein the hemoglobinopathy is selected from the group consisting of Thalassemia, Sickle Cell disease, vascular occlusion, beta-thalassemia, severe anemia due to intravascular hemolysis, and combinations thereof.

18. The method of claim 16, wherein the subject is in need of treatment for sickle cell disease.

19. The method of claim 16, wherein the composition decreases and/or inhibits ROS in sickle cell disease.

20. (canceled)

21. The method of claim 16, wherein the composition comprises the composition of claim 1.

22. (canceled)

23. The method of claim 16, wherein the composition further comprises an antisense oligonucleotide or a guide RNA specific for U32a.

24. (canceled)

25. The method of claim 16, wherein the composition further comprises an antisense oligonucleotide or a guide RNA specific for U35a.

26. (canceled)

27. (canceled)

28. (canceled)

29. The method of claim 16, wherein the composition comprises a guide RNA.

30. (canceled)

31. The method of claim 29, wherein the composition further comprises a guide RNA specific for at least one of U32a and U35a.

32. (canceled)

33. (canceled)

34. The method of claim 16, wherein the composition comprises an antisense oligonucleotide.

35. The method of claim 16, wherein the RPl13a snoRNA is selected from the group consisting of SEQ ID NOs: 49, 51 and 56.


SEQUENCE LISTING (57 sequences)

SEQ ID NO: 1 - DNA, 27 bp, Artificial Sequence
  Synthetic- Human  forward primer U32a
ggtcagtgatgagcaacattcaccatc

SEQ ID NO: 2 - DNA, 17 bp, Artificial Sequence
  Synthetic- Human  reverse primer U32a
tcccgaccaccacagcc

SEQ ID NO: 3 - DNA, 22 bp, Artificial Sequence
  Synthetic- Human forward primer  U33
ggccggtgatgagaacttctcc

SEQ ID NO: 4 - DNA, 17 bp, Artificial Sequence
  Synthetic- Human  reverse primer U33
tcccgaccaccacagcc

SEQ ID NO: 5 - DNA, 24 bp, Artificial Sequence
  Synthetic- Human  forward primer U34
cgtccatgatgttccgcaactacc

SEQ ID NO: 6 - DNA, 17 bp, Artificial Sequence
  Synthetic- Human  reverse primer  U34
tcccgaccaccacagcc

SEQ ID NO: 7 - DNA, 25 bp, Artificial Sequence
  Synthetic- Human forward primer  U35a
cttatctcacgatggtctgcggatg

SEQ ID NO: 8 - DNA, 17 bp, Artificial Sequence
  Synthetic- Human  reverse primer U35a
tcccgaccaccacagcc

SEQ ID NO: 9 - DNA, 20 bp, Artificial Sequence
  Synthetic- Human forward primer NOX1
atcctgcttcctgtgtgtcg

SEQ ID NO: 10 - DNA, 20 bp, Artificial Sequence
  Synthetic- Human reverse primer NOX1
gtcgtgtttcgggactggat

SEQ ID NO: 11 - DNA, 20 bp, Artificial Sequence
  Synthetic- Human forward primer NOX2
tgtcaagtgcccaaaggtgt

SEQ ID NO: 12 - DNA, 20 bp, Artificial Sequence
  Synthetic- Human reverse primer NOX2
cccaacgatgcggatatgga

SEQ ID NO: 13 - DNA, 20 bp, Artificial Sequence
  Synthetic- Human forward primer NOX4
cagtctttgaccctcggtcc

SEQ ID NO: 14 - DNA, 20 bp, Artificial Sequence
  Synthetic- Human reverse primer NOX4
tgatcctcggaggtaagcca

SEQ ID NO: 15 - DNA, 20 bp, Artificial Sequence
  Synthetic- Human forward primer GRK2
tccccatacccacaaggtca

SEQ ID NO: 16 - DNA, 20 bp, Artificial Sequence
  Synthetic- Human reverse primer GRK2
cttccactggcaaaaccgtg

SEQ ID NO: 17 - DNA, 20 bp, Artificial Sequence
  Synthetic- Human forward primer ERK1
gtcatcggcatccgagacat

SEQ ID NO: 18 - DNA, 18 bp, Artificial Sequence
  Synthetic- Human reverse primer ERK1
gactggcccacctcatcc

SEQ ID NO: 19 - DNA, 20 bp, Artificial Sequence
  Synthetic- Human forward primer ERK2
agttcttgacccctggtcct

SEQ ID NO: 20 - DNA, 20 bp, Artificial Sequence
  Synthetic- Human reverse primer ERK2
cctgggacatccccagaaac

SEQ ID NO: 21 - DNA, 21 bp, Artificial Sequence
  Synthetic- Human forward primer 36B4 (Rplp0)
ggagacggattacaccttccc

SEQ ID NO: 22 - DNA, 19 bp, Artificial Sequence
  Synthetic- Human reverse primer 36B4 (Rplp0)
cagccacaaaggcagatgg

SEQ ID NO: 23 - DNA, 25 bp, Artificial Sequence
  Synthetic- Mouse forward primer U32a
gagtccatgatcagcaacactcacc

SEQ ID NO: 24 - DNA, 17 bp, Artificial Sequence
  Synthetic- Mouse reverse primer U32a
tcccgaccaccacagcc

SEQ ID NO: 25 - DNA, 25 bp, Artificial Sequence
  Synthetic- Mouse forward primer U33
agcttgtgatgagacatctcccact

SEQ ID NO: 26 - DNA, 17 bp, Artificial Sequence
  Synthetic- Mouse reverse primer U33
tcccgaccaccacagcc

SEQ ID NO: 27 - DNA, 33 bp, Artificial Sequence
  Synthetic- Mouse forward primer U34
cgtctgtgatgttctgctattacctacattgtt

SEQ ID NO: 28 - DNA, 17 bp, Artificial Sequence
  Synthetic- Mouse reverse primer U34
tcccgaccaccacagcc

SEQ ID NO: 29 - DNA, 30 bp, Artificial Sequence
  Synthetic- Mouse forward primer U35a
ggcacatgatgttcttattctcacgatggt

SEQ ID NO: 30 - DNA, 17 bp, Artificial Sequence
  Synthetic- Mouse reverse primer U35a
tcccgaccaccacagcc

SEQ ID NO: 31 - DNA, 22 bp, Artificial Sequence
  Synthetic- Mouse forward primer NOX1
gaaattcttgggactgccttgg

SEQ ID NO: 32 - DNA, 28 bp, Artificial Sequence
  Synthetic- Mouse forward primer NOX2
tcaactactataaggtttatgatgatgg

SEQ ID NO: 33 - DNA, 27 bp, Artificial Sequence
  Synthetic- Mouse reverse primer NOX2
cagatatctaaattatgctcttccaaa

SEQ ID NO: 34 - DNA, 22 bp, Artificial Sequence
  Synthetic- Mouse forward primer NOX4
cctgctcatttggctgtcccta

SEQ ID NO: 35 - DNA, 22 bp, Artificial Sequence
  Synthetic- Mouse reverse primer NOX4
cggctacatgcacacctgagaa

SEQ ID NO: 36 - DNA, 18 bp, Artificial Sequence
  Synthetic- Mouse forward primer GRK2
ctgccagagcccagcatc

SEQ ID NO: 37 - DNA, 20 bp, Artificial Sequence
  Synthetic- Mouse reverse primer GRK2
tggcatgaggcctcttcttg

SEQ ID NO: 38 - DNA, 20 bp, Artificial Sequence
  Synthetic- Mouse forward primer ERK1
acactggctttctgacggag

SEQ ID NO: 39 - DNA, 20 bp, Artificial Sequence
  Synthetic- Mouse reverse primer ERK1
tgatgcgcttgtttgggttg

SEQ ID NO: 40 - DNA, 20 bp, Artificial Sequence
  Synthetic- Mouse forward primer ERK2
agccacacgttggtacagag

SEQ ID NO: 41 - DNA, 22 bp, Artificial Sequence
  Synthetic- Mouse reverse primer ERK2
acttacaccatctctcccttgc

SEQ ID NO: 42 - DNA, 21 bp, Artificial Sequence
  Synthetic- Mouse forward primer 36B4 (Rplp0)
atccctgacgcaccgccgtga

SEQ ID NO: 43 - DNA, 23 bp, Artificial Sequence
  Synthetic- Mouse reverse primer 36B4 (Rplp0)
tgcatctgcttggagcccacgtt

SEQ ID NO: 44 - DNA, 20 bp, Artificial Sequence
  Synthetic- Anti-sense oligo  U32a
  misc_feature (1)..(5): 2′-O-methoxyribonucleotide
  misc_feature (1)..(20): phosphorothioate backbone
  misc_feature (16)..(20): 2′-O-methoxyribonucleotide
gcggugcatgggttgaucuc

SEQ ID NO: 45 - DNA, 20 bp, Artificial Sequence
  Synthetic- Anti-sense oligo U33
  misc_feature (1)..(5): 2′ -O-methoxyribonucleotide
  misc_feature (1)..(20): phosphorothioate backbone
  misc_feature (16)..(20): 2′ -O-methoxyribonucleotide
ugguagtgcatgtagaguca

SEQ ID NO: 46 - DNA, 20 bp, Artificial Sequence
  Synthetic- Anti-sense oligo U35a
  misc_feature (1)..(5): 2′ -O-methoxyribonucleotide
  misc_feature (1)..(20): phosphorothioate backbone
  misc_feature (16)..(20): 2′ -O-methoxyribonucleotide
uuagcctttggcattaucgg

SEQ ID NO: 47 - DNA, 20 bp, Artificial Sequence
  Synthetic- Anti-sense oligo  U34
  misc_feature (1)..(5): 2′ -O-methoxyribonucleotide
  misc_feature (1)..(20): phosphorothioate backbone
  misc_feature (16)..(20): 2′ -O-methoxyribonucleotide
cagugggguuuucaugaggc

SEQ ID NO: 48 - DNA, 20 bp, Artificial Sequence
  Synthetic- Anti-sense oligo GFP
  misc_feature (1)..(5): 2′ -O-methoxyribonucleotide
  misc_feature (1)..(20): phosphorothioate backbone
  misc_feature (16)..(20): 2′ -O-methoxyribonucleotide
ucaccttcaccctctccacu

SEQ ID NO: 49 - DNA, 82 bp, Homo sapiens
  misc_feature (1)..(82): SNORD32A
aggtcagtgatgagcaacattcaccatctttcgtttgagtctcacggccatgagatcaaccccatgcaccgctctgagacct

SEQ ID NO: 50 - DNA, 83 bp, Homo sapiens
  misc_feature (1)..(83): SNORD33
ggccggtgatgagaacttctcccactcacattcgagtttcccgaccatgagatgactccacatgcactaccatctgaggccac

SEQ ID NO: 51 - DNA, 66 bp, Homo sapiens
  misc_feature (1)..(66): SNORD34
cgtccatgatgttccgcaactacctacattgtttgatcctcatgaaagcagcactggctgagacgc

SEQ ID NO: 52 - DNA, 81 bp, Mus musculus
  misc_feature (1)..(81): SNORD32a
gagtccatgatgagcaacactcaccatctttcgtttgagtctcacgactgtgagatcaacccatgcaccgctctgagactc

SEQ ID NO: 53 - DNA, 82 bp, Mus musculus
  misc_feature (1)..(82): SNORD33
agcttgtgatgagacatctcccactcatgttcgagttgctcgactatgagatgactctacatgcactaccatctgaggctgt

SEQ ID NO: 54 - DNA, 66 bp, Mus musculus
  misc_feature (1)..(65): SNORD34
cgtctgtgatgttctgctattacctacattgtttgagcctcatgaaaaccccactggctgagacgc

SEQ ID NO: 55 - DNA, 89 bp, Mus musculus
  misc_feature (1)..(89): SNORD35a
ggcacatgatgttcttattctcacgatggtcttcggatgccacagttagggcagtgccgataatgccaaaggctaagctgatgccagga

SEQ ID NO: 56 - DNA, 85 bp, Homo sapiens
  misc_feature (1)..(85): SNORD35a
ggcagatgatgtccttatctcacgatggtctgcggatgtccctgtgggaatggcgacaatgccaatggcttagctgatgccagga

SEQ ID NO: 57 - DNA, 22 bp, Artificial Sequence
  Synthetic- Mouse reverse primer NOX1
gctggagagaacagaagcgaga